(19) United States
(12) Patent Application Publication
(10) Pub. No.: US 2007/0249648 A1
(43) Pub. Date: Oct. 25, 2007

(54) Novel Diazaspiroalkanes and Their Use for Treatment of Ccr8 Mediated Diseases

(76) Inventors: Håkan Bladh, Lund (SE); Stephen Connolly, Leicestershire (GB); Hazel Joan Dyke, Harlow Essex (GB); Annea Lisius, Lund (SE); Stephen Price, Harlow Essex (GB); Igor Shamovsky, Lund (SE); Marco Van Den Heuvel, Harlow Essex (GB)

Correspondence Address: FISH & RICHARDSON P.C., P.O BOX 1022, MINNEAPOLIS, MN, 55440-1022, US

(21) Appl. No.: 10/575,525

(22) PCT Filed: Oct. 21, 2004
(86) PCT No.: PCT/SE04/01522

§ 371 Date: Mar. 29, 2007

(30) Foreign Application Priority Data
Oct. 23, 2003 (SE) 0302811-5

(51) Int. Cl. A61K 31/438; A61P 11/06; C07D 221/20

(52) U.S. Cl. 514/278; 546/16

(57) ABSTRACT

The invention provides compounds of general formula. wherein A, B, W, X, Y, Z, D, E, R1 and n are as defined in the specification, processes for their preparation, pharmaceutical compositions containing them and their use in therapy.

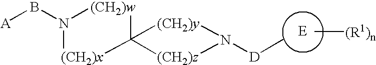

The present invention relates to a diazaspiro compound, processes and intermediates used in their preparation, pharmaceutical compositions containing them and their use in therapy.

Both the initial stages of a disease as well as the long-term tissue remodeling and muscle hypotrophy depend on recruitment of leukocytes to the inflammatory lesion. Leukocyte recruitment involves the migration of leukocytes into the diseased tissue from the blood vessel and their activation, which leads to progression of disease. The mechanism underlying this recruitment, chemotaxis, is similar both in classically defined immune mediated pathological conditions (i.e. allergic and autoimmune diseases) as well as others (i.e. atherosclerosis and Parkinson's disease). Thus, intervention of leukocyte recruitment to the inflamed target tissue constitutes an attractive novel therapeutic principle.

The chemokines are a large family (>50 members) of small 8- to 15-kDa secreted, heparin-binding polypeptides with the primary function of controlling trafficking and activation of leukocytes. They are distinct from classical chemoattractants (i.e. bacterial derived N-formyl peptides, complement components, lipid molecules and platelet activating factor) on the basis of shared structural similarities. All chemokines have four conserved cysteines residues that form disulfide bonds, which are critical for the 3-D structure. The chemokines are further subclassed according to the position of the first two cysteines. The two major subclasses are the CC-chemolines, that have the cysteines adjacent, and the CXC-cytokines, that have the cysteines separated by one amino acid. The two other families, the C and the CX3C chemokines, are much smaller and only comprise one or a few members.

The specific biological effects of chemokines, including leukocyte recruitment, are mediated via interactions with a family of seven-transmembrane G-protein coupled receptors (GPCRs). The chemokine receptors are ˜350 amino acids in length and consist of a short extracellular N-terminus, seven transmembrane segments, and an intracellular C-terminus. The seven transmembrane domains are α-helical, and 3 intracellular and 3 extracellular loops exist between the domains.

So far 18 human chemokine receptors have been identified. Of these there are 11 CC chemokine receptors, 5 CXC receptors, 1 CX3C receptor and 1 C receptor. In general, CC chemokines are potent chemoattractants of monocytes and lymphocytes, but poor activators of neutrophils. Certain receptors bind multiple chemoliines, for example, CCR1 binds CCL3, CCL5, CCL7 and CCL8, while other chemokine receptors have a more restricted binding profile. This ligand specificity, together with chemokine receptor expression patterns on particular leukocyte subsets, accounts for the regulated, restricted, and specific trafficking of cells into inflammatory lesions. Chemotaxis of inflammatory cells towards a chemokine gradient is initiated by signals mediated by the intracytoplasmatic tail of the chemokine receptor. The downstream signals involve the PI3Kγ, the MAPK and the PKC pathways, among others.

The accumulation of immune cells at a site of allergic inflammation occurs within 6-48 hours after allergen challenge and is a hallmark of allergic diseases. Studies have shown that antigen-specific CD4⁺ T cells are detected in lung tissue of in asthmatic patients after exposure to the allergen. Although infiltrating T cells are relatively few in number compared to eosinophils, compelling evidence has demonstrated essential roles for T cells in orchestrating the inflammatory process in human asthma. A close correlation exists in humans between the level of TH2 cytokines produced by T cells, serum level of IgE and prevalence of asthma.

The human CCR8 receptor has been shown to interact with the human chemokine CCL1 (I-309). This chemokine is a potent eosinophil, T cell and endothelial cell chemoattractant. The receptor has been shown to be transiently upregulated on polarized TH2 cells after optimal TCR cross linkage in presence of costimulatory signals (i.e. CD28). The coordinated upregulation of CCR8 on activated T cells after antigen challenge indicates that it contributes to redistribution of the activated T cells to the inflammatory foci within the inflamed tissue expressing CCL1. Indeed, in vivo models of allergic airway inflammation using mice deficient in CCR8 expression have shown a profound block in recruitment of effector T cells to the inflamed lung tissue and production of TH2 cytokines. Moreover, T cells infiltrating the human airway subepithelium during allergen challenge have been shown to be CCR8 positive. Importantly, the number of CCR8 positive cells migrating into the airway submucosa following allergen challenge has been shown to correlate with decreases in FEV1.

Considering the significant role CCR8 plays in TH2 cell chemotaxis, and the importance of TH2 cells in allergic conditions such as asthma, CCR8 represents a good target for drug development in treatment of asthma.

It has now been found that a series of diazaspiroundecanes have activity at the CCR8 receptor.

The present invention therefore provides compounds of formula (I) and pharmaceutically acceptable salts, solvates or N-oxides thereof: 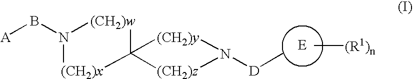
in which:
is w, x, y and z are independently 1, 2 or 3;
A is a phenyl, benzyl, alkyl, C₃₋₆ saturated or partially unsaturated cycloalkyl, a 6-membered-cycloheteroalkyl ring containing 1 or 2 heteroatoms selected from O or N, alkyl-aryl, naphthyl, a 5- to 7-membered heteroaromatic ring containing 1 to 3 heteroatoms, a 9- or 10-membered bicyclic heteroaromatic ring containing 1 to 4 heteroatoms, a phenyl-fused-5 to 6-membered cycloheteroalkyl containing at least one heteroatom selected from O, S or N, or pyridone;
A being optionally substituted by one or more groups selected from halogen, cyano, CF₃, OCF₃, C₁₋₆ alkoxy, hydroxy, C₁₋₆ alkyl, C₁₋₆ thioalkyl, SO₂C₁₋₆ alkyl, NR²R³, amide, C₁₋₆ alkoxycarbonyl, —NO₂, C₁₋₆ acylamino, —CO₂H, C₁₋₆ carboxyalkyl, morpholine;
phenoxy optionally substituted with one or more groups selected from halogen, C₁₋₆ alkoxy, C₁₋₆ alkyl;
phenyl or diphenyl, said phenyl and diphenyl independently being optionally substituted with one or more groups independently selected from halogen, C₁₋₆ alkoxy, C₁₋₆ alkyl, or —COOH;
benzyloxy optionally substituted with one or more groups selected from halogen, C₁₋₆ alkoxy, C₁₋₆ alkyl;
or a 5 to 7 membered heteroaromatic ring containing 1 to 4 heteroatoms selected from O, S or N optionally substituted with one or more groups independently selected from halogen, C₁₋₆ alkoxy, C₁₋₆ alkyl;
R² and R³ are independently C₁₋₆ alkyl, or R² and R³ together with the nitrogen to which they are attached form a 6-membered saturated ring optionally containing a further heteroatom;
B is a group R⁴-R⁵ where
R⁴ is a bond, —N(R⁶)—, —R⁷—N(R⁸)—, —N(R⁹)—R¹⁰—, O, C₁₋₄ alkyl optionally interrupted by N(R¹¹) or O, C₂₋₄ alkenyl or 1,3-butadienyl, or —SO₂—N(R¹²)—;
R⁵ is C═O or SO₂;
R⁶, R⁸, R¹¹, and R¹² are each independently H or C₁₋₆ alkyl;
R⁹ is H, C₁₋₆ alkyl or C₁₋₆ carboxyalkyl;
R⁷ and R¹⁰ are independently C₁₋₄ alkyl or C₃₋₅ cycloalkyl;
D is C₁₋₄ alkyl;
E is phenyl, or a 5- or 6-membered aromatic ring containing one or two heteroatoms;
Each R¹ independently represents C₁₋₆ alkoxy optionally substituted with one or more halogens, C₄₋₆ cycloalkylalkoxy, C₂₋₆ alkenyloxy, halogen, OCH₂CN, COC₁₋₆ alkyl, OR¹¹, OCH₂R¹¹, or —S—R¹²;
R¹¹ is a phenyl or 5- or 6-membered saturated or aromatic ring containing one or two heteroatoms and each optionally substituted by one or more groups selected from C₁₋₆ alkyl, halogen, C₁₋₆ alkoxy, CF₃, or cyano;
R¹² is C₁₋₆ alkyl or R¹² is phenyl optionally substituted with one or more halogens, and
n is 0, 1, 2, 3 or 4;
provided that when E is phenyl, w+x is greater than 2 and n is 1 then R¹ is not a phenoxy group at the meta-position of the phenyl ring E,
and provided that when A-B is acetyl, tosyl or tertiary butyloxy-carbonyl (t-boc), then D-E-(R¹)ₙ is not benzyl.

The present invention also provides compounds of formula (I′) and pharmaceutically acceptable salts, solvates or N-oxides thereof: 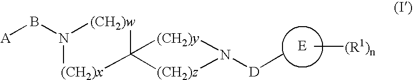
in which w, x, y, z, A, B, D, E, R¹, and n are as defined in formula (I), but with the proviso that when E is phenyl, and n is 1 then R¹ is not a phenoxy group at the meta-position of the phenyl ring E, and provided that when A-B is acetyl, tosyl or tertiary butyloxy-carbonyl (t-boc), then D-E-(R¹)ₙ is not benzyl.

Unless the context of the description otherwise describes, the following text relating to example chemical groups or preferred chemical groups applies to compounds of both formula (I) and formula (I′), and also formula (I″) (see below) insofar as it falls within the scope of formula's (1) and (I′).

Where the term “heteroatom” is used without being further defined in the context of its use, this term represents O, S or N (or when used in the plural form, any independent combination of O, S or N which corresponds to the number of heteroatoms mentioned).

The term alkyl, whether alone or as part of another group, includes straight chain and branched chain alkyl groups. Examples of 5- to 7-membered heteroaromatic rings containing 1 to 3 heteroatoms include thienyl, furanyl, pyrrolyl, imidazolyl, pyridyl, pyrazinyl, pyrimidyl, pyridazinyl, triazinyl, oxazolyl, thiazolyl, isoxazolyl, pyrazolyl, oxadiazolyl, thiadiazolyl, triazolyl and tetrazolyl. Examples of bicyclic 9- or 10-membered rings include indole, isoindole, indoline, benzofuran, benzothiophene, benzimidazole, benzthiazole, purine, quinoline, isoquinoline, cinnoline, quinazoline, quinoxaline, 1.8-naphthyridine. Substituents on any rings can be present in any suitable ring position including suitable substituents on nitrogen atoms. Aryl means phenyl or naphthyl.

w, x, y and z are independently 1, 2 or 3. In one embodiment, w+x is not greater than 4, and y+z is not greater than 4.

Example combinations of w+x, and y+z are listed below:

| w + x | | y + z |
|---|---|---|
| 4 | and | 4 |
| 3 | and | 4 |
| 4 | and | 3 |
| 2 | and | 4 |
| 4 | and | 2 |

When w+x is equal to 4, then both w and x may be equal to 2. Alternatively, one of w and x may be 1, and the other of w or x equal to 3.

When y+z is equal to 4, then both y and z may be equal to 2. Alternatively, one of y and z may be 1, and the other of y or z equal to 3.

When w+x is equal to 3, then one of w and x may be 1, and the other of w or x equal to 2.

When y+z is equal to 3, then one of y and z may be 1, and the other of y or z equal to 2.

In one embodiment of the invention, w and x are the same and y and z are the same, and x and y are independently 1 or 2.

In a further embodiment of the invention, w, x, y and z are equal to 2.

Combinations of w, x, y and z include: w, x, y and z each equal to 2; or w and x each equal to 2, one of y and z equal to 2 and the other of y and z equal to 1; or y and z each equal to 2, one of w and x equal to 2 and the other of w and x equal to 1; or w and x each equal to 1, and y and z each equal to 2.

A represents phenyl, benzyl, alkyl (e.g., methyl, ethyl, n-propyl, isopropyl, n-butyl, isobutyl, tert-butyl, n-pentyl or n-hexyl), C₃₋₆ saturated or partially unsaturated cycloalkyl (e.g., cyclopropyl, cyclobutyl, cyclopentyl or cyclohexyl), a 6-membered-cycloheteroalkyl ring containing 1 or 2 heteroatoms independently selected from O or N (e.g., tetrahydropyran or morpholine), alkyl-aryl, naphthyl, a 5- to 7-membered heteroaromatic ring containing 1 to 3 heteroatoms (e.g., thienyl, furanyl, pyrrolyl, imidazolyl, pyridyl, pyrazinyl, pyrimidyl, pyridazinyl, triazinyl, oxazolyl, thiazolyl, isoxazolyl, pyrazolyl, oxadiazolyl, thiadiazolyl, triazolyl and tetrazolyl), a 9 or 10-membered bicyclic heteroaromatic ring containing 1 to 4 heteroatoms (e.g., indole, isoindole, indoline, benzofuran, benzothiophene, benzimidazole, benzthiazole, purine, quinoline, isoquinoline, cinnoline, quinazoline, quinoxaline, or 1.8-naphthyridine), a phenyl-fused-5 to 6-membered cycloheteroalkyl containing at least one heteroatom selected from O, S or N, preferably containing 1 to 3 heteroatoms, more preferably 1 or 2 heteroatoms (e.g., benzodioxanyl, 3,4-dihydro-2H-1,3-benzoxazinyl, 1,3-benzodioxolyl, or 2,3 dihydrobenzofuranyl), pyridone or pyridine-N-oxide. When A is a phenyl-fused-5 to 6-membered cycloheteroalkyl containing at least one heteroatom selected from O, S or N, A is preferably connected to B through the phenyl group.

A may optionally be substituted by one or more groups selected from halogen (e.g., chlorine or fluorine), cyano, CF₃, OCF₃, C₁₋₆ alkoxy (e.g., methoxy, ethoxy, n-propoxy, i-propoxy, n-butoxy, i-butoxy, or t-butoxy), hydroxy, C₁₋₆ alkyl (e.g., methyl, ethyl, n-propyl, i-propyl, n-butyl, i-butyl, t-butyl, pentyl and hexyl), phenoxy, C₁₋₆ thioalkyl (e.g., methylthio-, ethylthio-, propylthio-, or butylthio-), SO₂C₁₋₆ alkyl (e.g., methylsulfonyl, or ethylsulfonyl), NR²R³, amide, C₁₋₆ alkoxycarbonyl (e.g., methoxycarbonyl or ethoxycarbonyl), —NO₂, C₁₋₆ acylamino (e.g., —NHCOCH₃), —CO₂H, C₁₋₆ carboxyalkyl (e.g., —(CH₂)n-COOH, where n is 1, 2, 3, 4, or 5), phenyl or diphenyl (said phenyl and diphenyl independently being optionally substituted with one or more groups selected from halogen such as chlorine or fluorine, C₁₋₆ alkoxy such as methoxy, C₁₋₆ alkyl such as methyl, or —COOH), benzyloxy (optionally substituted with one or more groups independently selected from halogen such as chlorine or fluorine, C₁₋₆ alkoxy such as methoxy, C₁₋₆ alkyl such as methyl), morpholine, or a 5 to 7 membered heteroaromatic ring containing 1 to 4 heteroatoms independently selected from O, S or N (e.g., oxazolyl, isoxazolyl, triazolyl, tetrazolyl, imidazolyl, or furanyl) optionally substituted with one or more groups independently selected from halogen such as chlorine or fluorine, C₁₋₆ alkoxy such as methoxy, or C₁₋₆ alkyl such as methyl.

R² and R³ are independently C₁₋₆ alkyl (e.g., methyl, ethyl, n-propyl, isopropyl, n-butyl, isobutyl, tert-butyl, n-pentyl or n-hexyl), or R² and R³ together with the nitrogen to which they are attached form a 6-membered saturated ring optionally containing a further heteroatom independently selected from O, S or N.

R⁴ is a bond, —N(R⁶)—, —R⁷—N(R⁸)—, —N(R⁹)—R¹⁰—, O, C₁₋₄ alkyl (e.g., -methyl, -ethyl, -propyl, -butyl) optionally interrupted by N(R¹¹) or O, C₂₋₄ alkenyl (e.g., -ethenyl, -propenyl) or 1,3-butadienyl, or —SO₂—N(R¹²)—.

R⁵ is C═O or SO₂.

R⁶, R⁸, R¹¹, and R² are each independently H or C₁₋₆ alkyl (e.g., methyl, ethyl, n-propyl, isopropyl, n-butyl, isobutyl, tert-butyl, n-pentyl or n-hexyl). Preferably, R⁶, R⁸, R¹¹, and R¹² are H.

R⁹ is H, C₁₋₆ alkyl (e.g., methyl, ethyl, n-propyl, isopropyl, n-butyl, isobutyl, tert-butyl, n-pentyl or n-hexyl), or C₁₋₆ carboxyalkyl (e.g., —(CH₂)n-COOH, where n is 1, 2, 3, 4, or 5).

R⁷ and R¹⁰ are independently C₁₋₄ alkyl (e.g., e.g., methyl, ethyl, propyl, butyl) or C₃₋₅ cycloalkylene (e.g., -cyclopropyl).

D is C₁₋₄ alkyl (e.g., -methyl, -ethyl, -propyl, or -butyl).

E is phenyl, a 5- or 6-membered aromatic ring containing one or two heteroatoms (e.g., pyridine, pyrimidine, thiophene, furan and pyrrole).

R¹ is C₁₋₆ alkoxy (e.g., methoxy, ethoxy, n-propoxy, i-propoxy, n-butoxy, i-butoxy, or t-butoxy) optionally substituted with one or more halogens (e.g., chlorine or fluorine, preferably fluorine), or R¹ is C₄₋₆ cycloalkylalkoxy (e.g., cyclopropylmethoxy), C₂₋₆ alkenyloxy (e.g., allyloxy, butenoxy, pentenoxy), halogen (e.g., chlorine or fluorine), OCH₂CN, COC₁₋₆ alkyl, OR¹¹, OCH₂R¹¹, or —S—R¹².

R¹¹ is a phenyl or a 5- or 6-membered saturated or aromatic ring containing one or two heteroatoms (e.g., isoxazolyl, thiazolyl tetrahydrofuranyl, tetrahydropyranyl, oxazolyl, isothiazolyl, imidazolyl, pyrazolyl, pyrrolinyl, pyrrolyl, thiophenyl and furanyl) and each optionally substituted by one or more groups (preferably 1 or 2 groups) independently selected from C₁₋₆ alkyl (such as methyl or ethyl, preferably methyl), halogen (e.g., chlorine or fluorine), C₁₋₆ alkoxy (e.g., methoxy), CF₃, or cyano.

R¹² is C₁₋₆ alkyl (e.g., methyl, ethyl, n-propyl, isopropyl, n-butyl, isobutyl, tert-butyl, n-pentyl or n-hexyl) or R¹² is phenyl optionally substituted with one or more halogens (e.g., chorine or fluorine).

When R² and R³ together with the nitrogen to which they are attached form a 6-membered saturated ring optionally containing a further heteroatom examples of such rings include morpholine and piperidine rings.

Preferably the ring A is phenyl, naphthyl, quinolyl, pyridyl or pyrimidyl each optionally substituted as defined above. Even more preferably, the ring A is phenyl or pyridyl. Preferred substituents include fluoro, chloro, methoxy, methyl, NMe₂, NEt₂, phenoxy, ethyl, propyl, t-butyl, thiomethyl, trifluoromethyl, cyano, butyloxy, ethoxy, propyloxy, morpholine, SO₂Me or C═OMe. In one embodiment of the invention, A is phenyl substituted by COOH or —CH₂—COOH. Preferably either a single substituent is present or two substituents are present on the ring A.

Preferably B is a group R⁴-R⁵ where R⁴ is a bond or —CH₂—, and R⁵ is C═O.

Preferably D is a —CH₂— group.

When E is a 5- or 6-membered aromatic ring containing one or two heteroatoms examples include pyridine, pyrimidine, thiophene, furan and pyrrole. Preferably E is phenyl or pyridyl. Most preferably, E is phenyl.

When R¹ is OR¹¹ or OCH₂R¹¹ where R¹¹ is a 5- or 6-membered saturated or aromatic ring containing one or two heteroatoms and optionally substituted by one or more C₁₋₆ alkyl groups, examples of suitable rings include tetrahydrofuran, tetrahydropyran, oxazole, isoxazolethiazole, isothiazole, imidazole, pyrazole, pyrroline, pyrrole, thiophene and furan.

In one embodiment, each R¹ independently represents C₁₋₆ alkoxy optionally substituted with one or more halogens, C₄₋₆ cycloalkylalkoxy, C₂₋₆ alkenyloxy, halogen, OCH₂CN, COC₁₋₆ alkyl, OR¹¹, OCH₂R¹¹, or —S—R¹²; where R¹¹ is a 5- or 6-membered saturated or aromatic ring containing one or two heteroatoms and each optionally substituted by one or more groups selected from C₁₋₆ alkyl, halogen, C₁₋₆ alkoxy, CF₃, or cyano; and R¹² is C₁₋₆ alkyl or R¹² is phenyl optionally substituted with one or more halogens

In one embodiment R¹ include —OCH₂CH═CH₂, butyloxy (preferably isobutyloxy), propyloxy, cyclopropylmethoxy, benzyloxy, ethoxy, bromo, methyl, chloro, OCH₂CN, fluoro, methoxy, CF₃, or OCH₂R¹¹ where R¹¹ is phenyl, tetrahydrofuran, tetrahydropyran, chlorothiazole or dimethyloxazole, or OR¹¹ where R¹¹ is phenyl.

Preferably n is 1 or 2, more preferably n is 1.

In one embodiment in formulas (I), (I′), and (I″), when E is phenyl or a 6-membered aromatic ring containing 1 or 2 heteroatoms, an R¹ group is present in an ortho position (i.e., 2-position) on ring E relative to D.

In a further embodiment in formulas (I), (I′), and (I″), when E is phenyl or a 6-membered aromatic ring containing 1 or 2 heteroatoms, and an R¹ group is phenoxy, the phenoxy is present in the ortho position on ring E relative to D.

In one embodiment in formulas (I), (I′), and (I″), when E is phenyl or a 6-membered aromatic ring containing 1 or 2 heteroatoms, an R¹ group is present in an ortho position on ring E relative to B and an R¹ group is not present in the meta position on ring E relative to D.

In one embodiment in formulas (I), (I′), and (I″), when E is phenyl or a 6-membered aromatic ring containing 1 or 2 heteroatoms, an R¹ group is present in a meta position on ring E relative to D (with the proviso in the case of formula (I) that when E is phenyl, w+x is greater than 2 and n is 1 then R¹ is not a phenoxy group at the meta-position of the phenyl ring E, and with the proviso in the case of formulas (I′) and (I″) that when E is phenyl, and n is 1 then R¹ is not a phenoxy group at the meta-position of the phenyl ring E).

In another embodiment, in formula (I), when w+x is less than 4 (for example, when w and x are both equal to 1), and when E is phenyl or a 6-membered heteroaromatic ring, an R¹ group is in an ortho position on ring E relative to D.

In another embodiment, in formula (I), when w+x is less than 4 (for example, when w and x are both equal to 1), and when E is phenyl or a 6-membered aromatic ring containing 1 or 2 heteroatoms, an R¹ group is in a meta position on ring E relative to D.

In one embodiment of the present invention, A in formulas (I) and (I′) is phenyl or pyridyl optionally substituted by one or two groups optionally selected from the group fluoro, chloro, methoxy, methyl, NMe₂, NEt₂, phenoxy, ethyl, propyl, t-butyl, thiomethyl, trifluoromethyl, cyano, butyloxy, ethoxy, propyloxy, morpholine, SO₂Me, C═OMe, COOH or —CH₂—COOH; w, x, y and z are independently 1, 2 or 3 and w+x is not greater than 4 and y+z is not greater than 4; B is —CH₂—C(═O)— or —C(═O)—; D is —CH₂—; E is phenyl or pyridyl; and one R¹ is methoxy, isobutyloxy, phenoxy, or cyclopropylmethoxy.

In another embodiment of the present invention, A in formulas (I) and (I′) is phenyl or pyridyl optionally substituted by one or two groups optionally selected from the group fluoro, chloro, methoxy, methyl, NMe₂, NEt₂, phenoxy, ethyl, propyl, t-butyl, thiomethyl, trifluoromethyl, cyano, butyloxy, ethoxy, propyloxy, morpholine, SO₂Me, C═OMe, COOH or —CH₂—COOH; w, x, y and z are independently 1, 2 or 3 and w+x is not greater than 4 and y+z is not greater than 4; B is —CH₂—C(═O)— or —C(═O)—; D is —CH₂—; E is phenyl or pyridyl; and one R¹ is methoxy, isobutoxy, phenoxy, or cyclopropylmethoxy in the ortho position of ring E.

In another embodiment of the present invention, A in formulas (I) and (I′) is phenyl or pyridyl optionally substituted by one or two groups optionally selected from the group fluoro, chloro, methoxy, methyl, NMe₂, NEt₂, phenoxy, ethyl, propyl, t-butyl, thiomethyl, trifluoromethyl, cyano, butyloxy, ethoxy, propyloxy, morpholine, SO₂Me, C═OMe, COOH or —CH₂—COOH; w, x, y and z are independently 1, 2 or 3 and w+x is not greater than 4 and y+z is not greater than 4; B is —CH₂—C(═O)— or —C(═O)—; D is —CH₂—; E is phenyl or pyridyl; and one R¹ is methoxy, isobutoxy, or cyclopropylmethoxy in the meta position of ring E.

In another aspect of the present invention, A in formulas (I) and (I′) is phenyl or pyridyl optionally substituted by one or two groups selected from the group fluoro, chloro, methoxy, methyl, NMe₂, NEt₂, phenoxy ethyl, propyl, t-butyl, thiomethyl, trifluoromethyl, cyano, butyloxy, ethoxy, propyloxy, morpholine, SO₂Me, C═OMe, COOH or —CH₂—COOH; w, x, y and z are independently 1, 2 or 3 and w+x is not greater than 4 and y+z is not greater than 4; B is —CH₂—C(═O)— or —C(═O)—; D is —CH₂—; E is phenyl or pyridyl; and one R¹ is isobutoxy or phenoxy in the ortho position of ring E.

The present invention also provides compounds of formula (I″) and pharmaceutically acceptable salts, solvates or N-oxides thereof: 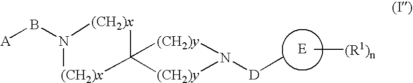
in which:
x and y are independently 1 or 2,
A is a phenyl, benzyl, alkyl, C₃₋₆ saturated or partially unsaturated cycloalkyl, alkyl-aryl naphthyl, a 5- to 7-membered heteroaromatic ring containing 1 to 3 heteroatoms, or a 9- or 10-membered bicyclic heteroaromatic ring containing 1 to 4 heteroatoms, each being optionally substituted by one or more groups selected from halogen, cyano, CF₃, OCF₃, C₁₋₆ alkoxy, C₁₋₆ alkyl, phenoxy, C₁₋₆ thioalkyl, SO₂C₁₋₆ alkyl, or NR²R³,
R² and R³ are independently halogen or C₁₋₆ alkyl or R² and R³ together with the nitrogen to which they are attached form a 6-membered saturated ring optionally containing a further heteroatom,
B is a group R⁴-R⁵ where R⁴ is a bond, NH, O or C₁₋₄ alkyl optionally interrupted by NH or O, and R⁵ is C═O or SO₂,
D is C₁₋₄ alkyl,
E is phenyl or a 5- or 6-membered aromatic ring containing one or two heteroatoms,
R¹ is C₂₋₆ alkoxy, C₂₋₆ alkenyloxy, phenoxy, benzyloxy, halogen, OCH₂CN, COC₁₋₆ alkyl, OR¹¹ or OCH₂R¹¹ where R¹¹ is phenyl, or a 5- or 6-membered saturated or aromatic ring containing one or two heteroatoms and optionally substituted by one or more C₁₋₆ alkyl groups,
and
n is 0, 1, 2, 3 or 4.
provided that when E is phenyl and n is 1 then R¹ is not a phenoxy group at the meta-position of the phenyl ring E.
In one embodiment, when A-B is acetyl, tosyl or tertiary butyloxy-carbonyl (t-boc), then D-E-(R¹)ₙ is not benzyl.

To the extent that groups A (and its substituents), R², R³, R⁴, R⁵, D, E, R¹, R¹¹ and n in formula (I″) are the same as those defined in formulas (I) and (I′), then the corresponding preferences and example groups referred to above in respect of formulas (I) and (I′) also apply to formula (I″).

In formula (I″) the term alkyl, whether alone or as part of another group, includes straight chain and branched chain alkyl groups. Examples of 5- to 7-membered heteroaromatic ring containing 1 to 3 heteroatoms include thienyl, furanyl, pyrrolyl, imidazolyl, pyridyl, pyrazinyl, pyrimidyl, pyridazinyl, triazinyl, oxazolyl, thiazolyl, isoxazolyl, pyrazolyl, oxadiazolyl, thiadiazolyl, triazolyl and tetrazolyl. Examples of bicyclic 9- or 10-membered rings include indole, isoindole, indoline, benzofuran, benzothiophene, benzimidazole, benzthiazole, purine, quinoline, isoquinoline, cinnoline, quinazoline, quinoxaline, 1.8-naphthyridine. Substituents on any rings can be present in any suitable ring position including suitable substituents on nitrogen atoms. Aryl means phenyl or naphthyl.

In formula (I″), when R² and R³ together with the nitrogen to which they are attached form a 6-membered saturated ring optionally containing a further heteroatom examples of such rings include morpholine and piperidine rings.

In formula (I″), preferably the ring A is phenyl, naphthyl, quinolyl or pyridyl each optionally substituted as defined above. Preferred substituents include chloro, methoxy, methyl, NMe₂, NEt₂, phenoxy, ethyl, propyl, t-butyl, thiomethyl, trifluoromethyl, cyano, butyloxy, ethoxy, propyloxy, morpholine, SO₂Me or C═OMe. Preferably either a single substituent is present or two substituents are present on the ring A.

In formula (I″), preferably B is a group R⁴-R⁵ where R⁴ is a bond and R⁵ is C═O.

In formula (I″), preferably D is a CH₂ group.

In formula (I″), when E is a 5- or 6-membered aromatic ring containing one or two heteroatoms examples include pyridine, pyrimidine, thiophene, furan and pyrrole. Preferably E is phenyl.

In formula (I″), when R¹ is OCH₂B where B is a 5- or 6-membered saturated or aromatic ring containing one or two heteroatoms and optionally substituted by one or more C₁₋₆ alkyl groups, examples of suitable rings include tetrahydrofuran, tetrahydropyran, oxazole, isoxazolethiazole, isothiazole, imidazole, pyrazole, pyrroline, pyrrole, thiophene and furan.

In formula (I″), preferred groups for R¹ include OCH₂CH═CH₂, butyloxy, propyloxy, benzyloxy, ethoxy, bromo, methyl, chloro, OCH₂CN, fluoro, methoxy, CF₃ or OCH₂R⁵ where R⁵ is tetrahydrofuran, tetrahydropyran or dimethyloxazole.

In formula (I″), preferably n is 1 or 2, more preferably n is 1.

Certain compounds of formulas (I), (I′) and (I″) are capable of existing in stereoisomeric forms. It will be understood that the invention encompasses all geometric and optical isomers of the compounds of formula (I), (I′) and (I″) and mixtures thereof including racemates. Tautomers and mixtures thereof also form an aspect of the present invention.

Preferred compounds the present invention include:
3-benzoyl-9-(2-ethoxybenzyl)-3,9-diazaspiro[5.5]undecane,
3-(2-ethoxybenzyl)-9-(4-ethylbenzoyl)-3,9-diazaspiro[5.5]undecane,
3-[(6-chloropyridin-3-yl)carbonyl]-9-(2-ethoxybenzyl)-3,9-diazaspiro[5.5]undecane,
(4-{[9-(2-ethoxybenzyl)-3,9-diazaspiro[5.5]undec-3-yl]carbonyl}phenyl)dimethylamine,
3-(2-ethoxybenzyl)-9-[2-methoxy-4-(methylthio)benzoyl]-3,9-diazaspiro[5.5]undecane,
3-(4-butoxybenzoyl)-9-(2-ethoxybenzyl)-3,9-diazaspiro[5.5]undecane,
1-(4-{[9-(2-ethoxybenzyl)-3,9-diazaspiro[5.5]undec-3-yl]carbonyl}phenyl)ethanone,
3-(2-ethoxybenzyl)-9-(quinolin-2-ylcarbonyl)-3,9-diazaspiro[5.5]undecane,
3-(2-ethoxybenzyl)-9-(3-phenoxybenzoyl)-3,9-diazaspiro[5.5]undecane,
3-(4-tert-butylbenzoyl)-9-(2-ethoxybenzyl)-3,9-diazaspiro[5.5]undecane,
4-{[9-(2-ethoxybenzyl)-3,9-diazaspiro[5.5]undec-3-yl]carbonyl}benzonitrile,
3-(2-ethoxybenzyl)-9-(6-methoxy-2-naphthoyl)-3,9-diazaspiro[5.5]undecane,
3-(2,3-dichlorobenzoyl)-9-(2-ethoxybenzyl)-3,9-diazaspiro[5.5]undecane,
3-(2-ethoxybenzyl)-9-(3-methoxybenzoyl)-3,9-diazaspiro[5.5]undecane,
3-(2,3-dimethylbenzoyl)-9-(2-ethoxybenzyl)-3,9-diazaspiro[5.5]undecane,
3-(4-chlorobenzoyl)-9-(2-ethoxybenzyl)-3,9-diazaspiro[5.5]undecane,
3-(2-ethoxybenzyl)-9-(4-methylbenzoyl)-3,9-diazaspiro[5.5]undecane,
3-(3,4-dichlorobenzoyl)-9-(2-ethoxybenzyl)-3,9-diazaspiro[5.5]undecane,
3-(3,4-dimethoxybenzoyl)-9-(2-ethoxybenzyl)-3,9-diazaspiro[5.5]undecane,
3-(2,4-dichlorobenzoyl)-9-(2-ethoxybenzyl)-3,9-diazaspiro[5.5]undecane,
3-(2-ethoxybenzyl)-9-(4-isopropoxybenzoyl)-3,9-diazaspiro[5.5]undecane,
3-(2-ethoxybenzyl)-9-(4-phenoxybenzoyl)-3,9-diazaspiro[5.5]undecane,
3-(2-ethoxybenzyl)-9-(2-naphthoyl)-3,9-diazaspiro[5.5]undecane,
3-(2-chlorobenzoyl)-9-(2-ethoxybenzyl)-3,9-diazaspiro[5.5]undecane,
3-(2,3-dimethoxybenzoyl)-9-(2-ethoxybenzyl)-3,9-diazaspiro[5.5]undecane,
3-(2-ethoxybenzyl)-9-(1-naphthoyl)-3,9-diazaspiro[5.5]undecane, (3-{[9-(2-ethoxybenzyl)-3,9-diazaspiro[5.5]undec-3-yl]carbonyl}phenyl)dimethylamine,
3-(2-ethoxybenzyl)-9-[3-(methylsulfonyl)benzoyl]-3,9-diazaspiro[5.5]undecane,
3-(2-ethoxybenzyl)-9-(4-methoxybenzoyl)-3,9-diazaspiro[5.5]undecane,
(4-{[9-(2-ethoxybenzyl)-3,9-diazaspiro[5.5]undec-3-yl]carbonyl}phenyl)diethylamine,
3-(2-ethoxybenzyl)-9-(4-propylbenzoyl)-3,9-diazaspiro[5.5]undecane,
3-(2-chloroisonicotinoyl)-9-(2-ethoxybenzyl)-3,9-diazaspiro[5.5]undecane,
3-(2-ethoxybenzyl)-9-[3-(trifluoromethyl)benzoyl]-3,9-diazaspiro[5.5]undecane,
3-(2-ethoxybenzyl)-9-[4-(trifluoromethyl)benzoyl]-3,9-diazaspiro[5.5]undecane,
3-(2-ethoxybenzyl)-9-(quinolin-4-ylcarbonyl)-3,9-diazaspiro[5.5]undecane,
3-(3-chloro-2-methylbenzoyl)-9-(2-ethoxybenzyl)-3,9-diazaspiro[5.5]undecane,
3-[2-(benzyloxy)benzyl]-9-[(6-chloropyridin-3-yl)carbonyl]-3,9-diazaspiro[5.5]undecane,
[4-({9-[2-(benzyloxy)benzyl]-3,9-diazaspiro[5.5]undec-3-yl}carbonyl)phenyl]dimethylamine,
3-[2-(benzyloxy)benzyl]-9-[2-methoxy-4-(methylthio)benzoyl]-3,9-diazaspiro[5.5]undecane,
1-[4-({9-[2-(benzyloxy)benzyl]-3,9-diazaspiro[5.5]undec-3-yl}carbonyl)phenyl]ethanone.
3-[2-(benzyloxy)benzyl]-9-(4-ethylbenzoyl)-3,9-diazaspiro[5.5]undecane,
3-[2-(benzyloxy)benzyl]-9-(quinolin-2-ylcarbonyl)-3,9-diazaspiro[5.5]undecane,
3-[2-(benzyloxy)benzyl]-9-(4-chloro-2-methoxybenzoyl)-3,9-diazaspiro[5.5]undecane,
3-({9-[2-(benzyloxy)benzyl]-3,9-diazaspiro[5.5]undec-3-yl}carbonyl)benzonitrile,
3-[2-(benzyloxy)benzyl]-9-(4-tert-butylbenzoyl)-3,9-diazaspiro[5.5]undecane,
4-({9-[2-(benzyloxy)benzyl]-3,9-diazaspiro[5.5]undec-3-yl}carbonyl)benzonitrile,
3-[2-(benzyloxy)benzyl]-9-(4-morpholin-4-ylbenzoyl)-3,9-diazaspiro[5.5]undecane,
3-[2-(benzyloxy)benzyl]-9-(2,3-dichlorobenzoyl)-3,9-diazaspiro[5.5]undecane,
3-[2-(benzyloxy)benzyl]-9-(3-methoxybenzoyl)-3,9-diazaspiro[5.5]undecane,
3-[2-(benzyloxy)benzyl]-9-(2,3-dimethylbenzoyl)-3,9-diazaspiro[5.5]undecane,
3-[2-(benzyloxy)benzyl]-9-(4-chlorobenzoyl)-3,9-diazaspiro[5.5]undecane,
3-[2-(benzyloxy)benzyl]-9-(4-methylbenzoyl)-3,9-diazaspiro[5.5]undecane,
3-[2-(benzyloxy)benzyl]-9-(3,4-dimethoxybenzoyl)-3,9-diazaspiro[5.5]undecane,
3-[2-(benzyloxy)benzyl]-9-(4-isopropoxybenzoyl)-3,9-diazaspiro[5.5]undecane,
3-[2-(benzyloxy)benzyl]-9-(4-phenoxybenzoyl)-3,9-diazaspiro[5.5]undecane,
3-[2-(benzyloxy)benzyl]-9-(2-naphthoyl)-3,9-diazaspiro[5.5]undecane,
3-[2-(benzyloxy)benzyl]-9-(2-chlorobenzoyl)-3,9-diazaspiro[5.5]undecane,
3-[2-(benzyloxy)benzyl]-9-(2,3-dimethoxybenzoyl)-3,9-diazaspiro[5.5]undecane,
3-[2-(benzyloxy)benzyl]-9-(1-naphthoyl)-3,9-diazaspiro[5.5]undecane,
[3-({9-[2-(benzyloxy)benzyl]-3,9-diazaspiro[5.5]undec-3-yl}carbonyl)phenyl]dimethylamine,
3-[2-(benzyloxy)benzyl]-9-(4-methoxybenzoyl)-3,9-diazaspiro[5.5]undecane,
[4-({9-[2-(benzyloxy)benzyl]-3,9-diazaspiro[5.5]undec-3-yl}carbonyl)phenyl]-diethylamine,
3-[2-(benzyloxy)benzyl]-9-(2-chloroisonicotinoyl)-3,9-diazaspiro[5.5]undecane,
3-[2-(benzyloxy)benzyl]-9-[3-(trifluoromethyl)benzoyl]-3,9-diazaspiro[5.5]undecane,
3-[2-(benzyloxy)benzyl]-9-[4-(trifluoromethyl)benzoyl]-3,9-diazaspiro[5.5]undecane,
3-[2-(benzyloxy)benzyl]-9-(quinolin-4-ylcarbonyl)-3,9-diazaspiro[5.5]undecane,
3-benzoyl-9-(2-propoxybenzyl)-3,9-diazaspiro[5.5]undecane,
3-benzoyl-9-[2-(tetrahydrofuran-2-ylmethoxy)benzyl]-3,9-diazaspiro[5.5]undecane,
3-(2-propoxybenzyl)-9-(pyridin-3-ylcarbonyl)-3,9-diazaspiro[5.5]undecane,
3-(2-isobutoxybenzyl)-9-(pyridin-4-ylcarbonyl)-3,9-diazaspiro[5.5]undecane,
3-(4-chlorobenzoyl)-9-[2-(pyridin-2-ylmethoxy)benzyl]-3,9-diazaspiro[5.5]undecane,
3-{[9-(2-isobutoxybenzyl)-3,9-diazaspiro[5.5]undec-3-yl]carbonyl}benzonitrile,
3-(2-isobutoxybenzyl)-9-(pyrazin-2-ylcarbonyl)-3,9-diazaspiro[5.5]undecane,
3-(2-isobutoxybenzyl)-9-(pyrimidin-2-ylcarbonyl)-3,9-diazaspiro[5.5]undecane,
3-(2-isobutoxybenzyl)-9-(pyrimidin-4-ylcarbonyl)-3,9-diazaspiro[5.5]undecane,
3-(2-isobutoxybenzyl)-9-(pyrimidin-5-ylcarbonyl)-3,9-diazaspiro[5.5]undecane,
3-(4-chlorobenzoyl)-9-[(6-isobutoxypyridin-2-yl)methyl]-3,9-diazaspiro[5.5]undecane,
2-(4-chlorobenzoyl)-7-(3-phenoxybenzyl)-2,7-diazaspiro[3.5]nonane,
2-benzoyl-7-(3-phenoxybenzyl)-2,7-diazaspiro[3.5]nonane,
3-(2-isobutoxybenzyl)-9-(pyridazin-3-ylcarbonyl)-3,9-diazaspiro[5.5]undecane,
3-(2-isobutoxybenzyl)-9-(pyridazin-4-ylcarbonyl)-3,9-diazaspiro[5.5]undecane,
3-(2-isobutoxybenzyl)-9-(pyridin-2-ylcarbonyl)-3,9-diazaspiro[5.5]undecane,
3-(2-isobutoxybenzyl)-9-(pyridin-3-ylcarbonyl)-3,9-diazaspiro[5.5]undecane,
3-(4-chlorobenzoyl)-9-[3-(pyridin-2-ylmethoxy)benzyl]-3,9-diazaspiro[5.5]undecane,
3-(4-chlorobenzoyl)-9-[3-(pyridin-3-ylmethoxy)benzyl]-3,9-diazaspiro[5.5]undecane,
3-(3-furoyl)-9-(2-isobutoxybenzyl)-3,9-diazaspiro[5.5]undecane,
3-(2-isobutoxybenzyl)-9-(3-thienylcarbonyl)-3,9-diazaspiro[5.5]undecane,
3-(4-chlorobenzoyl)-9-(2-isobutoxybenzyl)-3,9-diazaspiro[5.5]undecane,
3-benzoyl-9-(2-isobutoxybenzyl)-3,9-diazaspiro[5.5]undecane,
2-(3-furoyl)-8-(2-isobutoxybenzyl)-2,8-diazaspiro[4.5]decane,
2-{[8-(2-isobutoxybenzyl)-2,8-diazaspiro[4.5]dec-2-yl]carbonyl}quinoline,
2-{[2-(2-isobutoxybenzyl)-2,8-diazaspiro[4.5]dec-8-yl]carbonyl}quinoline,
8-(2-isobutoxybenzyl)-2-(pyridin-2-ylacetyl)-2,8-diazaspiro[4.5]decane,
2-(4-chlorobenzoyl)-7-(2-isobutoxybenzyl)-2,7-diazaspiro[3.5]nonane,
2-(4-chlorobenzoyl)-7-(2-phenoxybenzyl)-2,7-diazaspiro[3.5]nonane,
3-[(5-chloro-2-thienyl)carbonyl]-9-(2-isobutoxybenzyl)-3,9-diazaspiro[5.5]undecane,
3-(2-isobutoxybenzyl)-9-(1H-pyrrol-2-ylcarbonyl)-3,9-diazaspiro[5.5]undecane,
3-(2-isobutoxybenzyl)-9-[4-(1,3-oxazol-5-yl)benzoyl]-3,9-diazaspiro[5.5]undecane,
3-(2-isobutoxybenzyl)-9-[4-(1H-1,2,4-triazol-1-yl)benzoyl]-3,9-diazaspiro[5.5]undecane,
3-(4-chlorobenzoyl)-9-(3-isobutoxybenzyl)-3,9-diazaspiro[5.5]undecane,
3-(2-isobutoxybenzyl)-9-[(5-methyl-2-thienyl)carbonyl]-3,9-diazaspiro[5.5]undecane,
3-(4-chlorobenzoyl)-9-[(3-phenoxy-2-thienyl)methyl]-3,9-diazaspiro[5.5]undecane,
3-(2-isobutoxybenzyl)-9-[4-(trifluoromethyl)benzoyl]-3,9-diazaspiro[5.5]undecane,
3-[(6-chloropyridin-2-yl)carbonyl]-9-(2-isobutoxybenzyl)-3,9-diazaspiro[5.5]undecane,
3-(2-isobutoxybenzyl)-9-[(6-methylpyridin-3-yl)carbonyl]-3,9-diazaspiro[5.5]undecane,
3-[(6-chloropyridin-3-yl)carbonyl]-9-(2-isobutoxybenzyl)-3,9-diazaspiro[5.5]undecane,
3-(2-chloroisonicotinoyl)-9-(2-isobutoxybenzyl)-3,9-diazaspiro[5.5]undecane,
3-(2-isobutoxybenzyl)-9-(quinolin-2-ylcarbonyl)-3,9-diazaspiro[5.5]undecane,
2-[3-(4-chlorophenyl)propanoyl]-7-(2-phenoxybenzyl)-2,7-diazaspiro[3.5]nonane,
3-(2-isobutoxybenzyl)-9-[(1-oxidopyridin-3-yl)carbonyl]-3,9-diazaspiro[5.5]undecane,
3-[3-(pyridin-4-ylmethoxy)benzyl]-9-(pyrimidin-4-ylcarbonyl)-3,9-diazaspiro[5.5]undecane,
2-(4-chlorobenzoyl)-9-(2-isobutoxybenzyl)-2,9-diazaspiro[5.5]undecane,
9-(2-isobutoxybenzyl)-2-isonicotinoyl-2,9-diazaspiro[5.5]undecane,
2-(3-furoyl)-9-(2-isobutoxybenzyl)-2,9-diazaspiro[5.5]undecane,
9-(2-isobutoxybenzyl)-2-(quinolin-2-ylcarbonyl)-2,9-diazaspiro[5.5]undecane,
9-(2-isobutoxybenzyl)-2-(pyridin-4-ylacetyl)-2,9-diazaspiro[5.5]undecane,
7-(4-chlorobenzoyl)-2-(2-isobutoxybenzyl)-2,7-diazaspiro[4.5]decane,
2-(2-isobutoxybenzyl)-7-isonicotinoyl-2,7-diazaspiro[4.5]decane,
7-(3-furoyl)-2-(2-isobutoxybenzyl)-2,7-diazaspiro[4.5]decane,
2-{[2-(2-isobutoxybenzyl)-2,7-diazaspiro[4.5]dec-7-yl]carbonyl}quinoline,
2-(2-isobutoxybenzyl)-7-(pyridin-4-ylacetyl)-2,7-diazaspiro[4.5]decane,
2-(4-chlorobenzoyl)-7-(2-isobutoxybenzyl)-2,7-diazaspiro[4.4]nonane,
2-(2-isobutoxybenzyl)-7-isonicotinoyl-2,7-diazaspiro[4.4]nonane,
2-(3-furoyl)-7-(2-isobutoxybenzyl)-2,7-diazaspiro[4.4]nonane,
2-{[7-(2-isobutoxybenzyl)-2,7-diazaspiro[4.4]non-2-yl]carbonyl}quinoline,
2-(2-isobutoxybenzyl)-7-(pyridin-4-ylacetyl)-2,7-diazaspiro[4.4]nonane,
2-[(4-chlorophenyl)acetyl]-7-(2-isobutoxybenzyl)-2,7-diazaspiro[3.5]nonane,
2-[3-(4-chlorophenyl)propanoyl]-7-(3-phenoxybenzyl)-2,7-diazaspiro[3.5]nonane,
2-[3-(4-chlorophenyl)propanoyl]-7-(2-isobutoxybenzyl)-2,7-diazaspiro[3.5]nonane,
2-[(4-chlorophenyl)acetyl]-7-(2-isobutoxybenzyl)-2,7-diazaspiro[3.5]nonane,
2-(4-chlorobenzoyl)-7-(3-isobutoxybenzyl)-2,7-diazaspiro[4.4]nonane,
2-(4-chlorobenzoyl)-7-(2-phenoxybenzyl)-2,7-diazaspiro[4.4]nonane,
2-[2-(benzyloxy)benzyl]-7-(4-chlorobenzoyl)-2,7-diazaspiro[4.4]nonane,
3-(2-isobutoxybenzyl)-9-(quinolin-3-ylcarbonyl)-3,9-diazaspiro[5.5]undecane,
3-(2-isobutoxybenzyl)-9-(pyridin-4-ylacetyl)-3,9-diazaspiro[5.5]undecane,
8-(2-isobutoxybenzyl)-2-(pyridin-3-ylacetyl)-2,8-diazaspiro[4.5]decane,
8-(2-isobutoxybenzyl)-2-(pyridin-4-ylacetyl)-2,8-diazaspiro[4.5]decane,
7-(2-isobutoxybenzyl)-2-(pyridin-2-ylacetyl)-2,7-diazaspiro[3.5]nonane,
7-(2-isobutoxybenzyl)-2-(pyridin-3-ylacetyl)-2,7-diazaspiro[3.5]nonane,
8-(2-isobutoxybenzyl)-2-(pyridin-3-ylcarbonyl)-2,8-diazaspiro[4.5]decane,
8-(2-isobutoxybenzyl)-2-isonicotinoyl-2,8-diazaspiro[4.5]decane,
7-(2-isobutoxybenzyl)-2-(pyridin-4-ylacetyl)-2,7-diazaspiro[3.5]nonane,
8-(2-isobutoxybenzyl)-2-pyridin-2-ylcarbonyl)-2,8-diazaspiro[4.5]decane,
3-(2-isobutoxybenzyl)-9-(pyridin-2-ylacetyl)-3,9-diazaspiro[5.5]undecane,
3-(2-isobutoxybenzyl)-9-(pyridin-3-ylacetyl)-3,9-diazaspiro[5.5]undecane,
3-(2-isobutoxybenzyl)-9-[4-(2H-tetrazol-5-yl)benzoyl]-3,9-diazaspiro[5.5]undecane,
7-(2-isobutoxybenzyl)-2-(pyridin-2-ylcarbonyl)-2,7-diazaspiro[3.5]nonane,
7-(2-isobutoxybenzyl)-2-(pyridin-3-ylcarbonyl)-2,7-diazaspiro[3.5]nonane,
7-(2-isobutoxybenzyl)-2-isonicotinoyl-2,7-diazaspiro[3.5]nonane,
3-(2-isobutoxybenzyl)-9-(1-oxidoisonicotinoyl)-3,9-diazaspiro[5.5]undecane,
3-(2-isobutoxybenzyl)-9-(quinoxalin-2-ylcarbonyl)-3,9-diazaspiro[5.5]undecane,
3-[4-(1H-imidazol-1-yl)benzoyl]-9-(2-isobutoxybenzyl)-3,9-diazaspiro[5.5]undecane,
5-{[9-(2-isobutoxybenzyl)-3,9-diazaspiro[5.5]undec-3-yl]carbonyl}pyridin-2(1H)-one,
3-{[9-(2-isobutoxybenzyl)-3,9-diazaspiro[5.5]undec-3-yl]carbonyl}pyridin-2(1H)-one,
3-(2-isobutoxybenzyl)-9-[3-(2H-tetrazol-5-yl)benzoyl]-3,9-diazaspiro[5.5]undecane,
3-(2-isobutoxybenzyl)-9-(2-methylisonicotinoyl)-3,9-diazaspiro[5.5]undecane,
3-[2-(cyclopropylmethoxy)benzyl]-9-isonicotinoyl-3,9-diazaspiro[5.5]undecane,
3-[1-(2-isobutoxyphenyl)ethyl]-9-isonicotinoyl-3,9-diazaspiro[5.5]undecan,
3-[(6-isobutoxypyridin-2-yl)methyl]-9-isonicotinoyl-3,9-diazaspiro[5.5]undecane,
3-[(6-isobutoxypyridin-2-yl)methyl]-9-(pyrimidin-4-ylcarbonyl)-3,9-diazaspiro[5.5]undecane,
3-isonicotinoyl-9-{2-[(2-methylprop-2-en-1-yl)oxy]benzyl}-3,9-diazaspiro[5.5]undecane,
3-isonicotinoyl-9-(2-phenoxybenzyl)-3,9-diazaspiro[5.5]undecane,
3-(2-isobutoxybenzyl)-9-[2-(2H-tetrazol-5-yl)benzoyl]-3,9-diazaspiro[5.5]undecane,
3-isonicotinoyl-9-[2-(1,1,2,2-tetrafluoroethoxy)benzyl]-3,9-diazaspiro[5.5]undecane,
4-{[9-(2-isobutoxybenzyl)-3,9-diazaspiro[5.5]undecane-3-yl]carbonyl}benzene sulfonamide,
8-(2-isobutoxybenzyl)-2-(pyridin-2-ylacetyl)-2,8-diazaspiro[4.5]decane,
3-(4-chlorobenzoyl)-9-[(2-isobutoxypyridin-3-yl)methyl]-3,9-diazaspiro[5.5]undecane,
3-[(2-isobutoxypyridin-3-yl)methyl]-9-isonicotinoyl-3,9-diazaspiro[5.5]undecane,
3-[(2-isobutoxypyridin-3-yl)methyl]-9-(pyrimidin-4-ylcarbonyl)-3,9-diazaspiro[5.5]undecane,
9-(2-isobutoxybenzyl)-N-3-thienyl-3,9-diazaspiro[5.5]undecane-3-carboxamide,
N-(4-chlorophenyl)-9-(2-isobutoxybenzyl)-3,9-diazaspiro[5.5]undecane-3-carboxamide,
9-(2-isobutoxybenzyl)-N-(2-phenylethyl)-3,9-diazaspiro[5.5]undecane-3-carboxamide,
9-(2-isobutoxybenzyl)-N-[2-(2-thienyl)ethyl]-3,9-diazaspiro[5.5]undecane-3-carboxamide,
9-(2-isobutoxybenzyl)-N-2-thienyl-3,9-diazaspiro[5.5]undecane-3-carboxamide,
N-(2,3-dihydro-1-benzofuran-6-yl)-9-(2-isobutoxybenzyl)-3,9-diazaspiro[5.5]undecane-3-carboxamide,
N-(2,3-dihydro-1,4-benzodioxin-6-yl)-9-(2-isobutoxybenzyl)-3,9-diazaspiro[5.5]undecane-3-carboxamide,
9-(2-isobutoxybenzyl)-N-(5-methyl-3-phenylisoxazol-4-yl)-3,9-diazaspiro[5.5]undecane-3-carboxamide,
9-(2-isobutoxybenzyl)-N-(3-methyl-5-phenylisoxazol-4-yl)-3,9-diazaspiro[5.5]undecane-3-carboxamide,
N-(2,6-dichloropyridin-4-yl)-9-(2-isobutoxybenzyl)-3,9-diazaspiro[5.5]undecane-3-carboxamide,
N-2,1,3-benzothiadiazol-4-yl-9-(2-isobutoxybenzyl)-3,9-diazaspiro[5.5]undecane-3-carboxamide,
9-(2-isobutoxybenzyl)-N-(4-phenoxyphenyl)-3,9-diazaspiro[5.5]undecane-3-carboxamide,
9-(2-isobutoxybenzyl)-N-(2-phenylcyclopropyl)-3,9-diazaspiro[5.5]undecane-3-carboxamide,
9-(2-isobutoxybenzyl)-N-(tetrahydro-2H-pyran-2-yl)-3,9-diazaspiro[5.5]undecane-3-carboxamide,
9-(2-isobutoxybenzyl)-N-(phenyl)-3,9-diazaspiro[5.5]undecane-3-carboxamide,
N-benzyl-9-(2-isobutoxybenzyl)-3,9-diazaspiro[5.5]undecane-3-carboxamide,
N-cyclohexyl-9-(2-isobutoxybenzyl)-3,9-diazaspiro[5.5]undecane-3-carboxamide,
N-(tert-butyl)-9-(2-isobutoxybenzyl)-3,9-diazaspiro[5.5]undecane-3-carboxamide,
ethyl N-{[9-(2-isobutoxybenzyl)-3,9-diazaspiro[5.5]undec-3-yl]carbonyl}glycinate,
N-cyclopentyl-9-(2-isobutoxybenzyl)-3,9-diazaspiro[5.5]undecane-3-carboxamide,
N-(2,4-dichlorobenzyl)-9-(2-isobutoxybenzyl)-3,9-diazaspiro[5.5]undecane-3-carboxamide,
9-(2-isobutoxybenzyl)-N-(2-methoxyphenyl)-3,9-diazaspiro[5.5]undecane-3-carboxamide,
9-(2-isobutoxybenzyl)-N-(4-methoxyphenyl)-3,9-diazaspiro[5.5]undecane-3-carboxamide,
ethyl 4-({[9-(2-isobutoxybenzyl)-3,9-diazaspiro[5.5]undec-3-yl]carbonyl}amino)benzoate,
ethyl 3-({[9-(2-isobutoxybenzyl)-3,9-diazaspiro[5.5]undec-3-yl]carbonyl}amino)benzoate,
N-(3-chlorophenyl)-9-(2-isobutoxybenzyl)-3,9-diazaspiro[5.5]undecane-3-carboxamide,
9-(2-isobutoxybenzyl)-N-(4-methoxybenzyl)-3,9-diazaspiro[5.5]undecane-3-carboxamide,
N-[2-(4-ethylphenyl)ethyl]-9-(2-isobutoxybenzyl)-3,9-diazaspiro[5.5]undecane-3-carboxamide,
9-(2-isobutoxybenzyl)-N-(4-isopropylphenyl)-3,9-diazaspiro[5.5]undecane-3-carboxamide,
N-(3-cyanophenyl)-9-(2-isobutoxybenzyl)-3,9-diazaspiro[5.5]undecane-3-carboxamide,
9-(2-isobutoxybenzyl)-N-(2-methylphenyl)-3,9-diazaspiro[5.5]undecane-3-carboxamide,
9-(2-isobutoxybenzyl)-N-(3-methylphenyl)-3,9-diazaspiro[5.5]undecane-3-carboxamide,
9-(2-isobutoxybenzyl)-N-(4-methylphenyl)-3,9-diazaspiro[5.5]undecane-3-carboxamide,
N-(2,6-dichlorophenyl)-9-(2-isobutoxybenzyl)-3,9-diazaspiro[5.5]undecane-3-carboxamide,
N-(3,4-dichlorophenyl)-9-(2-isobutoxybenzyl)-3,9-diazaspiro[5.5]undecane-3-carboxamide,
N-(3,5-dichlorophenyl)-9-(2-isobutoxybenzyl)-3,9-diazaspiro[5.5]undecane-3-carboxamide,
N-(4-chlorophenyl)-9-(2-isobutoxybenzyl)-2,9-diazaspiro[5.5]undecane-2-carboxamide,
N-(4-chlorophenyl)-2-(2-isobutoxybenzyl)-2,7-diazaspiro[4.5]decane-7-carboxamide,
N-(4-chlorophenyl)-7-(2-isobutoxybenzyl)-2,7-diazaspiro[4.4]nonane-2-carboxamide,
N-(4-chlorophenyl)-7-(2-isobutoxybenzyl)-2,7-diazaspiro[3.5]nonane-2-carboxamide,
9-(2-isobutoxybenzyl)-N-[(4-methylphenyl)sulfonyl]-3,9-diazaspiro[5.5]undecane-3-carboxamide,
N-[(4-chlorophenyl)sulfonyl]-9-(2-isobutoxybenzyl)-3,9-diazaspiro[5.5]undecane-3-carboxamide,
9-(2-isobutoxybenzyl)-N-[(2-methylphenyl)sulfonyl]-3,9-diazaspiro[5.5]undecane-3-carboxamide,
N-[(4-chlorophenyl)sulfonyl]-9-(2-isobutoxybenzyl)-2,9-diazaspiro[5.5]undecane-2-carboxamide,
3-(2-isobutoxybenzyl)-9-(2-thienylsulfonyl)-3,9-diazaspiro[5.5]undecane,
3-(2-isobutoxybenzyl)-9-(phenylsulfonyl)-3,9-diazaspiro[5.5]undecane,
3-(2-isobutoxybenzyl)-9-(propylsulfonyl)-3,9-diazaspiro[5.5]undecane,
3-(2-isobutoxybenzyl)-9-[(3-methylphenyl)sulfonyl]-3,9-diazaspiro[5.5]undecane,
3-(benzylsulfonyl)-9-(2-isobutoxybenzyl)-3,9-diazaspiro[5.5]undecane,
3-(2-isobutoxybenzyl)-9-(isopropylsulfonyl)-3,9-diazaspiro[5.5]undecane,
3-(2-isobutoxybenzyl)-9-(3-thienylsulfonyl)-3,9-diazaspiro[5.5]undecane,
3-[(2,5-dimethyl-3-furyl)sulfonyl]-9-(2-isobutoxybenzyl)-3,9-diazaspiro[5.5]undecane,
3-[(3,5-dimethylisoxazol-4-yl)sulfonyl]-9-(2-isobutoxybenzyl)-3,9-diazaspiro[5.5]undecane,
2-{[9-(2-isobutoxybenzyl)-3,9-diazaspiro[5.5]undec-3-yl]sulfonyl}benzonitrile,
4-{[9-(2-isobutoxybenzyl)-3,9-diazaspiro[5.5]undec-3-yl]sulfonyl}benzonitrile,
3-[(2,5-dimethoxyphenyl)sulfonyl]-9-(2-isobutoxybenzyl)-3,9-diazaspiro[5.5]undecane,
3-(2-isobutoxybenzyl)-9-[(4-methoxyphenyl)sulfonyl]-3,9-diazaspiro[5.5]undecane,
3-(2-isobutoxybenzyl)-9-[(3-nitrophenyl)sulfonyl]-3,9-diazaspiro[5.5]undecane,
3-[(2-chlorophenyl)sulfonyl]-9-(2-isobutoxybenzyl)-3,9-diazaspiro[5.5]undecane,
3-[(4-chlorophenyl)sulfonyl]-9-(2-isobutoxybenzyl)-3,9-diazaspiro[5.5]undecane,
3-[(2,4-dimethyl-1,3-thiazol-5-yl)sulfonyl]-9-(2-isobutoxybenzyl)-3,9-diazaspiro[5.5]undecane,
3-(2,1,3-benzoxadiazol-4-ylsulfonyl)-9-(2-isobutoxybenzyl)-3,9-diazaspiro[5.5]undecane,
2-[(4-chlorophenyl)sulfonyl]-9-(2-isobutoxybenzyl)-2,9-diazaspiro[5.5]undecane,
7-[(4-chlorophenyl)sulfonyl]-2-(2-isobutoxybenzyl)-2,7-diazaspiro[4.5]decane,
2-[(4-chlorophenyl)sulfonyl]-7-(2-isobutoxybenzyl)-2,7-diazaspiro[4.4]nonane,
2-[(4-chlorophenyl)sulfonyl]-7-(2-isobutoxybenzyl)-2,7-diazaspiro[3.5]nonane,
3-(2-isobutoxybenzyl)-9-[(4-isopropylphenyl)sulfonyl]-3,9-diazaspiro[5.5]undecane,
4-{[9-(2-isobutoxybenzyl)-3,9-diazaspiro[5.5]undec-3-yl]sulfonyl}benzoic acid,
3-(2-isobutoxybenzyl)-9-(quinolin-8-ylsulfonyl)-3,9-diazaspiro[5.5]undecane,
3-[(5-chloro-1,3-dimethyl-1H-pyrazol-4-yl)sulfonyl]-9-(2-isobutoxybenzyl)-3,9-diazaspiro[5.5]undecane,
3-[(4-tert-butylphenyl)sulfonyl]-9-(2-isobutoxybenzyl)-3,9-diazaspiro[5.5]undecane,
N-(4-{[9-(2-isobutoxybenzyl)-3,9-diazaspiro[5.5]undec-3-yl]sulfonyl}phenyl)acetamide,
3-(2,1,3-benzothiadiazol-4-ylsulfonyl)-9-(2-isobutoxybenzyl)-3,9-diazaspiro[5.5]undecane,
2-hydroxy-5-{[9-(2-isobutoxybenzyl)-3,9-diazaspiro[5.5]undec-3-yl]sulfonyl}benzoic acid,
methyl 3-{[9-(2-isobutoxybenzyl)-3,9-diazaspiro[5.5]undec-3-yl]sulfonyl}thiophene-2-carboxylate,
3-{[4-(2-furyl)phenyl]sulfonyl}-9-(2-isobutoxybenzyl)-3,9-diazaspiro[5.5]undecane,
3-(2-isobutoxybenzyl)-9-[(4-methyl-3,4-dihydro-2H-1,4-benzoxazin-7-yl)sulfonyl]-3,9-diazaspiro[5.5]undecane,
3-(2-isobutoxybenzyl)-9-[(5-methyl-1-phenyl-1H-pyrazol-4-yl)sulfonyl]-3,9-diazaspiro[5.5]undecane,
3-(2-isobutoxybenzyl)-9-[(6-morpholin-4-ylpyridin-3-yl)sulfonyl]-3,9-diazaspiro[5.5]undecane,
3-(2,3-dihydro-1-benzofuran-5-ylsulfonyl)-9-(2-isobutoxybenzyl)-3,9-diazaspiro[5.5]undecane,
3-{[9-(2-isobutoxybenzyl)-3,9-diazaspiro[5.5]undec-3-yl]carbonyl}benzoic acid,
4-{2-[9-(2-isobutoxybenzyl)-3,9-diazaspiro[5.5]undec-3-yl]-2-oxoethyl}benzoic acid,
2-{[9-(2-isobutoxybenzyl)-3,9-diazaspiro[5.5]undec-3-yl]carbonyl}benzoic acid,
(2-{[9-(2-isobutoxybenzyl)-3,9-diazaspiro[5.5]undec-3-yl]carbonyl}phenyl)acetic acid,
(3-{[9-(2-isobutoxybenzyl)-3,9-diazaspiro[5.5]undec-3-yl]carbonyl}phenyl)acetic acid,
[{2-[9-(2-isobutoxybenzyl)-3,9-diazaspiro[5.5]undec-3-yl]-2-oxoethyl}(phenyl)amino]acetic acid,
5-{[9-(2-isobutoxybenzyl)-3,9-diazaspiro[5.5]undec-3-yl]carbonyl}thiophene-2-carboxylic acid,
(2E,4E)-6-[9-(2-isobutoxybenzyl)-3,9-diazaspiro[5.5]undec-3-yl]-6-oxohexa-2,4-dienoic acid,
6-[9-(2-isobutoxybenzyl)-3,9-diazaspiro[5.5]undec-3-yl]-6-oxohexanoic acid,
4′-{[9-(2-isobutoxybenzyl)-3,9-diazaspiro[5.5]undec-3-yl]carbonyl}biphenyl-4-carboxylic acid,
(3-{2-[9-(2-isobutoxybenzyl)-3,9-diazaspiro[5.5]undec-3-yl]-2-oxoethyl}phenyl)acetic acid,
3-{[9-(2-isobutoxybenzyl)-3,9-diazaspiro[5.5]undec-3-yl]carbonyl}-1H-pyrazole-5-carboxylic acid,
{2-[9-(2-isobutoxybenzyl)-3,9-diazaspiro[5.5]undec-3-yl]-2-oxoethoxy}acetic acid,
3-(4-chlorobenzoyl)-9-{2-[(2,6-dichlorobenzyl)oxy]benzyl}-3,9-diazaspiro[5.5]undecane,
3-(4-chlorobenzoyl)-9-[2-(2-methoxyphenoxy)benzyl]-3,9-diazaspiro[5.5]undecane,
3-[2-(tert-butylthio)benzyl]-9-(4-chlorobenzoyl)-3,9-diazaspiro[5.5]undecane,
3-(4-chlorobenzoyl)-9-[3-(pyridin-2-yloxy)benzyl]-3,9-diazaspiro[5.5]undecane,
3-(4-chlorobenzoyl)-9-[(3,5-diethoxypyridin-4-yl)methyl]-3,9-diazaspiro[5.5]undecane,
2-(2-{[9-(4-chlorobenzoyl)-3,9-diazaspiro[5.5]undec-3-yl]methyl}phenoxy)benzonitrile,
3-[2-(allyloxy)benzyl]-9-(4-chlorobenzoyl)-3,9-diazaspiro[5.5]undecane,
3-[3-(benzyloxy)benzyl]-9-(4-chlorobenzoyl)-3,9-diazaspiro[5.5]undecane,
3-(4-chlorobenzoyl)-9-(4-phenoxybenzyl)-3,9-diazaspiro[5.5]undecane,
3-(4-chlorobenzoyl)-9-[2-(4-methylphenoxy)benzyl]-3,9-diazaspiro[5.5]undecane,
3-[2-(4-tert-butylphenoxy)benzyl]-9-(4-chlorobenzoyl)-3,9-diazaspiro[5.5]undecane,
3-(4-chlorobenzoyl)-9-[2-(3-chlorophenoxy)benzyl]-3,9-diazaspiro[5.5]undecane,
3-(4-chlorobenzoyl)-9-[2-(4-fluorophenoxy)benzyl]-3,9-diazaspiro[5.5]undecane,
3-(4-chlorobenzoyl)-9-{2-[3-(trifluoromethyl)phenoxy]benzyl}-3,9-diazaspiro[5.5]undecane,
3-(4-chlorobenzoyl)-9-[2-(2,4-dichlorophenoxy)benzyl]-3,9-diazaspiro[5.5]undecane,
3-(4-chlorobenzoyl)-9-{2-[(2-fluorophenyl)thio]benzyl}-3,9-diazaspiro[5.5]undecane,
3-(4-chlorobenzoyl)-9-(4-fluoro-3-phenoxybenzyl)-3,9-diazaspiro[5.5]undecane,
3-(4-chlorobenzoyl)-9-(2-isobutoxybenzyl)-3,9-diazaspiro[5.5]undecane,
2-[2-(allyloxy)benzyl]-7-(4-chlorobenzoyl)-2,7-diazaspiro[3.5]nonane,
7-(4-chlorobenzoyl)-2-[2-(3-chlorophenoxy)benzyl]-2,7-diazaspiro[3.5]nonane,
7-(4-chlorobenzoyl)-2-[2-(4-fluorophenoxy)benzyl]-2,7-diazaspiro[3.5]nonane,
7-(4-chlorobenzoyl)-2-{2-[3-(trifluoromethyl)phenoxy]benzyl}-2,7-diazaspiro[3.5]nonane,
2-(2-{[7-(4-chlorobenzoyl)-2,7-diazaspiro[3.5]non-2-yl]methyl}phenoxy)benzonitrile,
7-(4-chlorobenzoyl)-2-[2-(pyridin-3-yloxy)benzyl]-2,7-diazaspiro[3.5]nonane,
7-(4-chlorobenzoyl)-2-(4-fluoro-3-phenoxybenzyl)-2,7-diazaspiro[3.5]nonane,
7-(4-chlorobenzoyl)-2-(2-isobutoxybenzyl)-2,7-diazaspiro[3.5]nonane,
7-[2-(allyloxy)benzyl]-2-(4-chlorobenzoyl)-2,7-diazaspiro[3.5]nonane,
7-[3-(benzyloxy)benzyl]-2-(4-chlorobenzoyl)-2,7-diazaspiro[3.5]nonane,
2-(4-chlorobenzoyl)-7-[2-(3-chlorophenoxy)benzyl]-2,7-diazaspiro[3.5]nonane,
2-(4-chlorobenzoyl)-7-[2-(4-fluorophenoxy)benzyl]-2,7-diazaspiro[3.5]nonane,
2-(4-chlorobenzoyl)-7-{2-[3-(trifluoromethyl)phenoxy)benzyl}-2,7-diazaspiro[3.5]nonane,
2-(2-{(2-(4-chlorobenzoyl)-2,7-diazaspiro[3.5]non-7-yl]methyl}phenoxy)benzonitrile,
2-(4-chlorobenzoyl)-7-[2-(pyridin-3-yloxy)benzyl]-2,7-diazaspiro[3.5]nonane,
2-(4-chlorobenzoyl)-7-(4-fluoro-3-phenoxybenzyl)-2,7-diazaspiro[3.5]nonane,
2-(4-chlorobenzoyl)-7-(2-isobutoxybenzyl)-2,7-diazaspiro[3.5]nonane,
8-[2-(allyloxy)benzyl]-2-(4-chlorobenzoyl)-2,8-diazaspiro[4.5]decane,
8-[3-(benzyloxy)benzyl]-2-(4-chlorobenzoyl)-2,8-diazaspiro[4.5]decane,
2-(4-chlorobenzoyl)-8-(4-phenoxybenzyl)-2,8-diazaspiro[4.5]decane,
2-(4-chlorobenzoyl)-8-[2-(3-chlorophenoxy)benzyl]-2,8-diazaspiro[4.5]decane,
2-(4-chlorobenzoyl)-8-[2-(4-fluorophenoxy)benzyl]-2,8-diazaspiro[4.5]decane,
2-(4-chlorobenzoyl)-8-{2-[3-(trifluoromethyl)phenoxy]benzyl}-2,8-diazaspiro[4.5]decane,
2-(4-chlorobenzoyl)-8-[2-(2,4-dichlorophenoxy)benzyl]-2,8-diazaspiro[4.5]decane,
2-(2-{[2-(4-chlorobenzoyl)-2,8-diazaspiro[4.5]dec-8-yl]methyl}phenoxy)benzonitrile,
2-(4-chlorobenzoyl)-8-(4-fluoro-3-phenoxybenzyl)-2,8-diazaspiro[4.5]decane,
2-(4-chlorobenzoyl)-8-(2-isobutoxybenzyl)-2,8-diazaspiro[4.5]decane,
2-[2-(allyloxy)benzyl]-8-(4-chlorobenzoyl)-2,8-diazaspiro[4.5]decane,
2-[3-(benzyloxy)benzyl]-8-(4-chlorobenzoyl)-2,8-diazaspiro[4.5]decane,
8-(4-chlorobenzoyl)-2-[2-(3-chlorophenoxy)benzyl]-2,8-diazaspiro[4.5]decane,
8-(4-chlorobenzoyl)-2-[2-(4-fluorophenoxy)benzyl]-2,8-diazaspiro[4.5]decane,
8-(4-chlorobenzoyl)-2-{2-[3-(trifluoromethyl)phenoxy]benzyl}-2,8-diazaspiro[4.5]decane,
2-(2-{[8-(4-chlorobenzoyl)-2,8-diazaspiro[4.5]dec-2-yl]methyl}phenoxy)benzonitrile,
8-(4-chlorobenzoyl)-2-{2-[(2-chloro-1,3-thiazol-5-yl)methoxy]benzyl}-2,8-diazaspiro[4.5]decane,
8-(4-chlorobenzoyl)-2-(4-fluoro-3-phenoxybenzyl)-2,8-diazaspiro[4.5]decane,
8-(4-chlorobenzoyl)-2-(2-isobutoxybenzyl)-2,8-diazaspiro[4.5]decane,
3-(4-chlorobenzoyl)-9-[2-(3-methylbutoxy)benzyl]-3,9-diazaspiro[5.5]undecane,
3-(2-ethoxybenzyl)-9-(4-fluorobenzoyl)-3,9-diazaspiro[5.5]undecane,
3-(2-ethoxybenzyl)-9-(4-nitrobenzoyl)-3,9-diazaspiro[5.5]undecane,
3-(4-chlorobenzoyl)-9-(2-isobutoxybenzyl)-3,9-diazaspiro[5.5]undecane,
3-(2-isobutoxybenzyl)-9-(4-nitrobenzoyl)-3,9-diazaspiro[5.5]undecane,
3-(4-fluorobenzoyl)-9-(2-isobutoxybenzyl)-3,9-diazaspiro[5.5]undecane,
2-chloro-5-{[9-(2-isobutoxybenzyl)-3,9-diazaspiro[5.5]undec-3-yl]carbonyl}benzenesulfonamide,
3-(2-isobutoxybenzyl)-9-(1H-pyrrol-3-ylcarbonyl)-3,9-diazaspiro[5.5]undecane,
8-(2-isobutoxybenzyl)-2-[2-(methylsulfonyl)benzoyl]-2,8-diazaspiro[4.5]decane,
2-[4-chloro-2-(methylsulfonyl)benzoyl]-g-(2-isobutoxybenzyl)-2,8-diazaspiro[4.5]decane,
2-{[8-(2-isobutoxybenzyl)-2,8-diazaspiro[4.5]dec-2-yl]carbonyl}nicotinamide,
8-(2-isobutoxybenzyl)-2-[(2-morpholin-4-ylpyridin-3-yl)carbonyl]-2,8-diazaspiro[4.5]decane,
2-[(2,6-dimethoxypyridin-3-yl)carbonyl]-8-(2-isobutoxybenzyl)-2,8-diazaspiro[4.5]decane,
2-(2,4-dimethoxybenzoyl)-8-(2-isobutoxybenzyl)-2,8-diazaspiro[4.5]decane,
3-[(4-chlorobenzyl)sulfonyl]-9-(2-ethoxybenzyl)-3,9-diazaspiro[5.5]undecane,
8-(2-isobutoxybenzyl)-2-[4-(methylsulfonyl)benzoyl]-2,8-diazaspiro[4.5]decane,
8-(2-isobutoxybenzyl)-2-[2-methoxy-4-(methylthio)benzoyl]-2,8-diazaspiro[4.5]decane,
2-(4-butoxybenzoyl)-8-(2-isobutoxybenzyl)-2,8-diazaspiro[4.5]decane,
1-(4-{[8-(2-isobutoxybenzyl)-2,8-diazaspiro[4.5]dec-2-yl]carbonyl}phenyl)ethanone,
2-(4-ethylbenzoyl)-8-(2-isobutoxybenzyl)-2,8-diazaspiro[4.5]decane,
2-{[8-(2-isobutoxybenzyl)-2,8-diazaspiro[4.5]dec-2-yl]carbonyl}quinoline,
2-(4-chloro-2-methoxybenzoyl)-8-(2-isobutoxybenzyl)-2,8-diazaspiro[4.5]decane,
8-(2-isobutoxybenzyl)-2-(4-morpholin-4-ylbenzoyl)-2,8-diazaspiro[4.5]decane,
8-(2-isobutoxybenzyl)-2-(6-methoxy-2-naphthoyl)-2,8-diazaspiro[4.5]decane,
2-(2,3-dichlorobenzoyl)-8-(2-isobutoxybenzyl)-2,8-diazaspiro[4.5]decane,
8-(2-isobutoxybenzyl)-2-(3-methoxybenzoyl)-2,8-diazaspiro[4.5]decane,
2-(2,3-dimethylbenzoyl)-8-(2-isobutoxybenzyl)-2,8-diazaspiro[4.5]decane,
8-(2-isobutoxybenzyl)-2-(4-methylbenzoyl)-2,8-diazaspiro[4.5]decane,
2-(3,4-dichlorobenzoyl)-8-(2-isobutoxybenzyl)-2,8-diazaspiro[4.5]decane,
2-(2,4-dichlorobenzoyl)-8-(2-isobutoxybenzyl)-2,8-diazaspiro[4.5]decane,
8-(2-isobutoxybenzyl)-2-(4-isopropoxybenzoyl)-2,8-diazaspiro[4.5]decane,
8-(2-isobutoxybenzyl)-2-(4-phenoxybenzoyl)-2,8-diazaspiro[4.5]decane,
8-(2-isobutoxybenzyl)-2-(2-naphthoyl)-2,8-diazaspiro[4.5]decane,
2-(2-chlorobenzoyl)-8-(2-isobutoxybenzyl)-2,8-diazaspiro[4.5]decane,
2-(2,3-dimethoxybenzoyl)-8-(2-isobutoxybenzyl)-2,8-diazaspiro[4.5]decane,
8-(2-isobutoxybenzyl)-2-(1-naphthoyl)-2,8-diazaspiro[4.5]decane,
8-(2-isobutoxybenzyl)-2-(4-methoxybenzoyl)-2,8-diazaspiro[4.5]decane,
N,N-diethyl-4-{[8-(2-isobutoxybenzyl)-2,8-diazaspiro[4.5]dec-2-yl]carbonyl}aniline,
8-(2-isobutoxybenzyl)-2-(4-propylbenzoyl)-2,8-diazaspiro[4.5]decane,
8-(2-isobutoxybenzyl)-2-[3-(trifluoromethyl)benzoyl]-2,8-diazaspiro[4.5]decane,
8-(2-isobutoxybenzyl)-2-[4-(trifluoromethyl)benzoyl]-2,8-diazaspiro[4.5]decane,
4-{[8-(2-isobutoxybenzyl)-2,8-diazaspiro[4.5]dec-2-yl]carbonyl}quinoline,
2-(3-chloro-2-methylbenzoyl)-8-(2-isobutoxybenzyl)-2,8-diazaspiro[4.5]decane,
(4-{2-[8-(2-isobutoxybenzyl)-2,8-diazaspiro[4.5]dec-2-yl]-2-oxoethyl}phenyl)dimethylamine,
2-[(2-fluorophenyl)acetyl]-8-(2-isobutoxybenzyl)-2,8-diazaspiro[4.5]decane,
8-(2-isobutoxybenzyl)-2-[(3-nitrophenyl)acetyl]-2,8-diazaspiro[4.5]decane,
8-(2-isobutoxybenzyl)-2-[(4-nitrophenyl)acetyl]-2,8-diazaspiro[4.5]decane,
8-(2-isobutoxybenzyl)-2-[(2-nitrophenyl)acetyl]-2,8-diazaspiro[4.5]decane,
2-[(3,4-dimethoxyphenyl)acetyl]-8-(2-isobutoxybenzyl)-2,8-diazaspiro[4.5]decane,
2-(3-furoyl)-8-(2-isobutoxybenzyl)-2,8-diazaspiro[4.5]decane,
2-[(4-chlorophenyl)acetyl]-8-(2-isobutoxybenzyl)-2,8-diazaspiro[4.5]decane,
8-(2-isobutoxybenzyl)-2-(1,2,3-thiadiazol-4-ylcarbonyl)-2,8-diazaspiro[4.5]decane,
8-(2-isobutoxybenzyl)-2-[(5-methyl-1H-pyrazol-3-yl)carbonyl]-2,8-diazaspiro[4.5]decane,
2-[(1,5-dimethyl-1H-pyrazol-3-yl)carbonyl]-8-(2-isobutoxybenzyl)-2,8-diazaspiro[4.5]decane,
2-[(4-butoxyphenyl)acetyl]-8-(2-isobutoxybenzyl)-2,8-diazaspiro[4.5]decane,
2-[(3,5-difluorophenyl)acetyl]-8-(2-isobutoxybenzyl)-2,8-diazaspiro[4.5]decane,
2-[(2,4-dichlorophenyl)acetyl]-8-(2-isobutoxybenzyl)-2,8-diazaspiro[4.5]decane,
2-[(2,4-difluorophenyl)acetyl]-8-(2-isobutoxybenzyl)-2,8-diazaspiro[4.5]decane,
8-(2-isobutoxybenzyl)-2-[(3-methylisoxazol-4-yl)carbonyl]-2,8-diazaspiro[4.5]decane,
8-(2-isobutoxybenzyl)-2-(2-methyl-3-furoyl)-2,8-diazaspiro[4.5]decane,
8-(2-isobutoxybenzyl)-2-[(5-methylisoxazol-4-yl)carbonyl]-2,8-diazaspiro[4.5]decane,
2-(1,3-benzodioxol-5-ylacetyl)-8-(2-isobutoxybenzyl)-2,8-diazaspiro[4.5]decane,
2-[(3,5-dimethylisoxazol-4-yl)carbonyl]-8-(2-isobutoxybenzyl)-2,8-diazaspiro[4.5]decane,
8-(2-isobutoxybenzyl)-2-[(1,2,5-trimethyl-1H-pyrrol-3-yl)carbonyl]-2,8-diazaspiro[4.5]decane,
8-(2-isobutoxybenzyl)-2-[(5-nitro-1H-pyrazol-3-yl)carbonyl]-2,8-diazaspiro[4.5]decane,
2-[(2,5-difluorophenyl)acetyl]-g-(2-isobutoxybenzyl)-2,8-diazaspiro[4.5]decane,
2-{[4-(benzyloxy)-3-methoxyphenyl]acetyl}-8-(2-isobutoxybenzyl)-2,8-diazaspiro[4.5]decane,
8-(2-isobutoxybenzyl)-2-{[4-(trifluoromethoxy)phenyl]acetyl}-2,8-diazaspiro[4.5]decane,
2-(2,5-dimethyl-3-furoyl)-8-(2-isobutoxybenzyl)-2,8-diazaspiro[4.5]decane,
8-(2-isobutoxybenzyl)-2-[(4-methylphenyl)acetyl]-2,8-diazaspiro[4.5]decane,
8-(2-isobutoxybenzyl)-2-[(4-isopropylphenyl)acetyl]-2,8-diazaspiro[4.5]decane,
8-(2-isobutoxybenzyl)-2-{[4-(methylsulfonyl)phenyl]acetyl}-2,8-diazaspiro[4.5]decane,
8-(2-isobutoxybenzyl)-2-(1H-pyrazol-4-ylcarbonyl)-2,8-diazaspiro[4.5]decane,
8-(2-isobutoxybenzyl)-2-[(4-nitro-1H-pyrazol-3-yl)carbonyl]-2,8-diazaspiro[4.5]decane,
(2-{[8-(2-isobutoxybenzyl)-2,8-diazaspiro[4.5]dec-2-yl]carbonyl}phenyl)dimethylamine,
2-[(3,5-dimethylphenyl)acetyl]-8-(2-isobutoxybenzyl)-2,8-diazaspiro[4.5]decane,
2-(3-chloro-4-methylbenzoyl)-8-(2-isobutoxybenzyl)-2,8-diazaspiro[4.5]decane,
8-(2-isobutoxybenzyl)-2-[(4-methoxy-3-thienyl)carbonyl]-2,8-diazaspiro[4.5]decane,
8-(2-isobutoxybenzyl)-2-{[3-(trifluoromethyl)phenyl]acetyl}-2,8-diazaspiro[4.5]decane,
8-[(6-chloropyridin-3-yl)carbonyl]-2-(2-isobutoxybenzyl)-2,8-diazaspiro[4.5]decane,
(4-{[2-(2-isobutoxybenzyl)-2,8-diazaspiro[4.5]dec-8-yl]carbonyl}phenyl)dimethylamine,
2-(2-isobutoxybenzyl)-8-[4-(methylsulfonyl)benzoyl]-2,8-diazaspiro[4.5]decane,
8-(4-butoxybenzoyl)-2-(2-isobutoxybenzyl)-2,8-diazaspiro[4.5]decane,
1-(4-{[2-(2-isobutoxybenzyl)-2,8-diazaspiro[4.5]dec-8-yl]carbonyl}phenyl)ethanone,
8-(4-ethylbenzoyl)-2-(2-isobutoxybenzyl)-2,8-diazaspiro[4.5]decane,
2-{[2-(2-isobutoxybenzyl)-2,8-diazaspiro[4.5]dec-8-yl]carbonyl}quinoline,
8-(4-chloro-2-methoxybenzoyl)-2-(2-isobutoxybenzyl)-2,8-diazaspiro[4.5]decane,
3-{[2-(2-isobutoxybenzyl)-2,8-diazaspiro[4.5]dec-8-yl]carbonyl}benzonitrile,
2-(2-isobutoxybenzyl)-8-(3-phenoxybenzoyl)-2,8-diazaspiro[4.5]decane,
8-(4-tert-butylbenzoyl)-2-(2-isobutoxybenzyl)-2,8-diazaspiro[4.5]decane,
4-{[2-(2-isobutoxybenzyl)-2,8-diazaspiro[4.5]dec-8-yl]carbonyl}benzonitrile,
2-(2-isobutoxybenzyl)-8-(4-morpholin-4-ylbenzoyl)-2,8-diazaspiro[4.5]decane,
2-(2-isobutoxybenzyl)-8-(6-methoxy-2-naphthoyl)-2,8-diazaspiro[4.5]decane,
8-(2,3-dichlorobenzoyl)-2-(2-isobutoxybenzyl)-2,8-diazaspiro[4.5]decane,
2-(2-isobutoxybenzyl)-8-(3-methoxybenzoyl)-2,8-diazaspiro[4.5]decane,
8-(2,3-dimethylbenzoyl)-2-(2-isobutoxybenzyl)-2,8-diazaspiro[4.5]decane,
2-(2-isobutoxybenzyl)-8-(4-methylbenzoyl)-2,8-diazaspiro[4.5]decane,
8-(3,4-dichlorobenzoyl)-2-(2-isobutoxybenzyl)-2,8-diazaspiro[4.5]decane,
8-(3,4-dimethoxybenzoyl)-2-(2-isobutoxybenzyl)-2,8-diazaspiro[4.5]decane,
8-(2,4-dichlorobenzoyl)-2-(2-isobutoxybenzyl)-2,8-diazaspiro[4.5]decane,
2-(2-isobutoxybenzyl)-8-(4-isopropoxybenzoyl)-2,8-diazaspiro[4.5]decane,
2-(2-isobutoxybenzyl)-8-(2-naphthoyl)-2,8-diazaspiro[4.5]decane,
8-(2-chlorobenzoyl)-2-(2-isobutoxybenzyl)-2,8-diazaspiro[4.5]decane,
8-(2,3-dimethoxybenzoyl)-2-(2-isobutoxybenzyl)-2,8-diazaspiro[4.5]decane,
2-(2-isobutoxybenzyl)-8-(1-naphthoyl)-2,8-diazaspiro[4.5]decane,
(3-{[2-(2-isobutoxybenzyl)-2,8-diazaspiro[4.5]dec-8-yl]carbonyl}phenyl)dimethylamine,
2-(2-isobutoxybenzyl)-8-(4-methoxybenzoyl)-2,8-diazaspiro[4.5]decane,
N,N-diethyl-4-{[2-(2-isobutoxybenzyl)-2,8-diazaspiro[4.5]dec-8-yl]carbonyl}aniline,
2-(2-isobutoxybenzyl)-8-(4-propylbenzoyl)-2,8-diazaspiro[4.5]decane,
8-(2-chloroisonicotinoyl)-2-(2-isobutoxybenzyl)-2,8-diazaspiro[4.5]decane,
2-(2-isobutoxybenzyl)-8-[3-(trifluoromethyl)benzoyl]-2,8-diazaspiro[4.5]decane,
2-(2-isobutoxybenzyl)-8-[4-(trifluoromethyl)benzoyl]-2,8-diazaspiro[4.5]decane,
4-{[2-(2-isobutoxybenzyl)-2,8-diazaspiro[4.5]dec-8-yl]carbonyl}quinoline,
8-(3-chloro-2-methylbenzoyl)-2-(2-isobutoxybenzyl)-2,8-diazaspiro[4.5]decane,
(4-{2-[2-(2-isobutoxybenzyl)-2,8-diazaspiro[4.5]dec-8-yl]-2-oxoethyl}phenyl)dimethylamine,
8-[(2-fluorophenyl)acetyl]-2-(2-isobutoxybenzyl)-2,8-diazaspiro[4.5]decane,
2-(2-isobutoxybenzyl)-8-[(3-nitrophenyl)acetyl]-2,8-diazaspiro[4.5]decane,
2-(2-isobutoxybenzyl)-8-[(4-nitrophenyl)acetyl]-2,8-diazaspiro[4.5]decane,
2-(2-isobutoxybenzyl)-8-[(2-nitrophenyl)acetyl]-2,8-diazaspiro[4.5]decane,
8-[(3,4-dimethoxyphenyl)acetyl]-2-(2-isobutoxybenzyl)-2,8-diazaspiro[4.5]decane,
8-(3-furoyl)-2-(2-isobutoxybenzyl)-2,8-diazaspiro[4.5]decane,
8-[(4-chlorophenyl)acetyl]-2-(2-isobutoxybenzyl)-2,8-diazaspiro[4.5]decane,
2-(2-isobutoxybenzyl)-8-(1,2,3-thiadiazol-4-ylcarbonyl)-2,8-diazaspiro[4.5]decane,
2-(2-isobutoxybenzyl)-8-[(5-methyl-1H-pyrazol-3-yl)carbonyl]-2,8-diazaspiro[4.5]decane,
8-[(1,5-dimethyl-1H-pyrazol-3-yl)carbonyl]-2-(2-isobutoxybenzyl)-2,8-diazaspiro[4.5]decane,
8-[(4-butoxyphenyl)acetyl]-2-(2-isobutoxybenzyl)-2,8-diazaspiro[4.5]decane,
8-[(3,5-difluorophenyl)acetyl]-2-(2-isobutoxybenzyl)-2,8-diazaspiro[4.5]decane,
8-[(2,4-dichlorophenyl)acetyl]-2-(2-isobutoxybenzyl)-2,8-diazaspiro[4.5]decane,
8-[(2,4-difluorophenyl)acetyl]-2-(2-isobutoxybenzyl)-2,8-diazaspiro[4.5]decane,
2-(2-isobutoxybenzyl)-8-[(3-methylisoxazol-4-yl)carbonyl]-2,8-diazaspiro[4.5]decane,
2-(2-isobutoxybenzyl)-8-(2-methyl-3-furoyl)-2,8-diazaspiro[4.5]decane,
2-(2-isobutoxybenzyl)-8-[(5-methylisoxazol-4-yl)carbonyl]-2,8-diazaspiro[4.5]decane,
8-(1,3-benzodioxol-5-ylacetyl)-2-(2-isobutoxybenzyl)-2,8-diazaspiro[4.5]decane,
2-(2-isobutoxybenzyl)-8-[(1,2,5-trimethyl-1H-pyrrol-3-yl)carbonyl]-2,8-diazaspiro[4.5]decane,
2-(2-isobutoxybenzyl)-8-[(5-nitro-1H-pyrazol-3-yl)carbonyl]-2,8-diazaspiro[4.5]decane,
8-[(2,5-difluorophenyl)acetyl]-2-(2-isobutoxybenzyl)-2,8-diazaspiro[4.5]decane,
8-{[4-(benzyloxy)-3-methoxyphenyl]acetyl}-2-(2-isobutoxybenzyl)-2,8-diazaspiro[4.5]decane,
2-(2-isobutoxybenzyl)-8-{[4-(trifluoromethoxy)phenyl]acetyl}-2,8-diazaspiro[4.5]decane,
8-(2,5-dimethyl-3-furoyl)-2-(2-isobutoxybenzyl)-2,8-diazaspiro[4.5]decane,
2-(2-isobutoxybenzyl)-8-[(4-methylphenyl)acetyl]-2,8-diazaspiro[4.5]decane,
2-(2-isobutoxybenzyl)-8-(3-thienylcarbonyl)-2,8-diazaspiro[4.5]decane,
2-(2-isobutoxybenzyl)-8-(pyridin-4-ylacetyl)-2,8-diazaspiro[4.5]decane,
2-(2-isobutoxybenzyl)-8-(pyridin-2-ylacetyl)-2,8-diazaspiro[4.5]decane,
2-(2-isobutoxybenzyl)-8-[(4-isopropylphenyl)acetyl]-2,8-diazaspiro[4.5]decane,
2-(2-isobutoxybenzyl)-8-{[4-(methylsulfonyl)phenyl]acetyl}-2,8-diazaspiro[4.5]decane,
2-(2-isobutoxybenzyl)-8-(1H-pyrazol-4-ylcarbonyl)-2,8-diazaspiro[4.5]decane,
2-(2-isobutoxybenzyl)-8-[(4-nitro-1H-pyrazol-3-yl)carbonyl]-2,8-diazaspiro[4.5]decane,
(2-{[2-(2-isobutoxybenzyl)-2,8-diazaspiro[4.5]dec-8-yl]carbonyl}phenyl)dimethylamine,
   and pharmaceutically acceptable salts and solvates thereof.

According to the present invention there is also provided a process for the preparation of compounds of formula (I), (I′) and (I″) which comprises:
(a) reaction of a compound of formula (I): 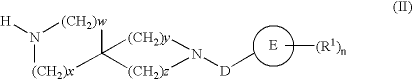
where R¹, n, D, E, w, x, y and z are as defined in formulae (I) or (I′) or are protected derivatives thereof, or a compound of formula (II′) 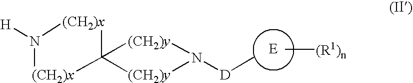
where R¹, n, D, E, x and y are as defined in formulae (I″) or are protected derivatives thereof,
with a compound of formula (III):
A-B-LG (III)
where A and B are as defined in formulae (I), (I′) or (I″) or are protected derivatives thereof and LG is a leaving group, or
(b) for compounds of formula (I), (I′) or (I″) where R⁴ is N and R⁵ is C═O, reaction of a compound of formula (II) or (II′) as defined above with a compound of formula (IV):
AN═C═O (IV)
in which A is as defined in formula (I), (I′) or (I″) or is a protected derivative thereof and optionally thereafter (a) or (b):
removing any protecting groups,
forming a pharmaceutically acceptable salt.

When B is a group R⁴-R⁵ where R⁴ is a bond and R⁵ is C═O, then the group LG is preferably OH. The reaction can be carried out in the presence of a base such as DEA with HBTU in a suitable solvent such as NMP.

When B is a group R⁴-R⁵ where R⁴ is O or a bond and R⁵ is C═O or SO₂, then the group LO is preferably Cl.

When B is a group R⁴-R⁵ where R⁴ is N and R⁵ is SO₂, then the group LG is preferably Cl.

Reaction of a compound of formula (II) or (II′) with a isocyanate of formula AN═C═O can be carried out in the presence of a suitable solvent at a suitable temperature (such as room temperature).

A compound of formula (II) or (II′) can be prepared by reaction of a compound of formula (V) or (V′) respectively 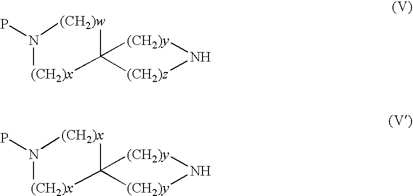
in which w, x, y and z are as defined in formulas (I), (I′) or (I″) and P is a protecting group, with an aldehyde compound of formula (VI): 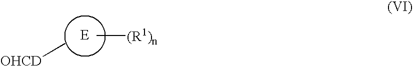
in which E, R¹ and n are as defined in formulas (I), (I′) or (I″) or are protected derivatives thereof and D is alkyl or a bond. The reaction can be carried out in the presence of NaB(OAc)₃H in DMF/HOAca t ambient temperature. The protecting group P is suitably a group such as CO₂Bu¹.

It will be appreciated by those skilled in the art that in the processes of the present invention certain functional groups such as hydroxyl or amino groups in the starting reagents or intermediate compound may need to be protected by protecting groups. Thus, the preparation of the compounds of formulas (I), (I′) and (I″) may involve, at an appropriate stage, the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991).

The compounds of formulas (I), (I′) and (I″) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate or p-toluenesulphonate.

The compounds of formulas (I), (I′) and (I″) have activity as pharmaceuticals, in particular as modulators of chemokine receptor (especially CCR8) activity, and may be used in the treatment (therapeutic or prophylactic) of conditions/diseases in human and non-human animals which are exacerbated or caused by excessive or dysregulated production of chemokines. Examples of such conditions/diseases include:
(1) (the respiratory tract) obstructive airways diseases including chronic obstructive pulmonary disease (COPD), asthma, such as bronchial, allergic, intrinsic, extrinsic and dust asthma, particularly chronic or inveterate asthma (e.g. late asthma and airways hyper-responsiveness), bronchitis, acute, allergic, atrophic rhinitis and chronic rhinitis including rhinitis caseosa, hypertrophic rhinitis, rhinitis purulenta, rhinitis sicca and rhinitis medicamentosa, membranous rhinitis including croupous, fibrinous and pseudomembranous rhinitis and scrofoulous rhinitis, seasonal rhinitis including rhinitis nervosa (hay fever) and vasomotor rhinitis, sarcoidosis, farmer's lung and related diseases, fibroid lung and idiopathic interstitial pneumonia,
(2) (bone and joints) gout, rheumatoid arthritis, seronegative spondyloarthropathies (including ankylosing spondylitis, psoriatic arthritis and Reiter's disease), Behcet's disease, Sjogren's syndrome and systemic sclerosis,
(3) (skin) pruritis, scleroderma, otitus, psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis, lupus,
(4) (gastrointestinal tract) Coeliac disease, proctitis, eosinopilic gastro-enteritis, mastocytosis, inflammatory bowel diseases such as Crohn's disease, ulcerative colitis, ileitis and enteritis, food-related allergies which have effects remote from the gut, e.g., migraine, rhinitis and eczema,
(5) (central and peripheral nervous system) Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia, polyneuropathies, e.g. Guillain-Barré syndrome, chronic inflammatory demyelinating polyradiculoneuropathy, multifocal motor neuropathy, plexopathies, CNS demyelination, e.g. multiple sclerosis, acute disseminated/haemorrhagic encephalomyelitis, and subacute sclerosing panencephalitis, neuromuscular disorders, e.g. myasthenia gravis and Lambert-Eaton syndrome, spinal disorders, e.g. tropical spastic paraparesis, and stiff-man syndrome: paraneoplastic syndromes, e.g. cerebellar degeneration and encephalomyelitis, CNS trauma, migraine, stroke and correctum diseases such as meningitis
(6) (other tissues and systemic disease) hepatitis, vasculitis, spondyloarthopathies, vaginitis, glomerulonephritis, myositis, atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura, post-operative adhesions, and sepsis.
(7) (allograft and xenograft rejection) acute and chronic following, for example, transplantation of kidney, heart, liver, lung, bone marrow, skin and cornea, and chronic graft versus host disease,
(8) Cancer, carcinoma & tumour metastasis, including that of the bladder, breast, colon, kidney, liver, lung, ovary, pancreas, stomach, cervix, thyroid and skin, especially non-small cell lung cancer (NSCLC), malignant melanoma, prostate cancer and squamous sarcoma. Hematopoietic tumors of lymphoid lineage, including acute lymphocytic leukemia, B cell lymphoma and Burketts lymphoma, Hodgkins Lymphoma, Acute Lymphoblastic Leukemia. Hematopoietic tumors of myeloid lineage, including acute and chronic myelogenous leukemias and promyelocytic leukemia. Tumors of mesenchymal origin, including fibrosarcoma and rhabdomyosarcoma, and other tumors, including melanoma, seminoma, tetratocarcinoma, neuroblastoma and glioma.
(9) All diseases that result from a general inbalance of the immune system and resulting in increased atopic inflammatory reactions.
(10) Cystic fibrosis, re-perfusion injury in the heart, brain, peripheral limbs and other organs.
(11) Burn wounds & chronic skin ulcers
(12) Reproductive Diseases (e.g. Disorders of ovulation, menstruation and implantation, Pre-term labour, Endometriosis)
(13) thrombosis
(14) infectious diseases such as HIV infection and other viral infections, bacterial infections.

Thus, the present invention provides a compound of formula (I), (I′) or (I″), or a pharmaceutically-acceptable salt or solvates thereof, as hereinbefore defined for use in therapy.

Preferably the compounds of the invention are used to treat diseases in which the chemokine receptor belongs to the CC chemokine receptor subfamily, more preferably the target chemokine receptor is the CCR8 receptor.

Particular conditions which can be treated with the compound of the invention are asthma, rhinitis and inflammatory skin disorders, diseases in which there are raised I-309, TARC, or MDC levels. It is preferred that the compound of the invention is used to treat asthma and rhinitis, especially asthma.

In a further aspect, the present invention provides the use of a compound of formula (I), (I′) or (I″), or a pharmaceutically acceptable salt or solvate thereof, as hereinbefore defined in the manufacture of a medicament for use in therapy.

In a still further aspect, the present invention provides the use of a compound of formula (I), (I′) or (I″), or a pharmaceutically acceptable salt or solvate thereof, as hereinbefore defined in the manufacture of a medicament for the treatment of human diseases or conditions in which modulation of chemokine receptor activity, particularly CCR8 activity, is beneficial.

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CCR8) receptor, which comprises administering to a patient a therapeutically effective amount of a compound of formula (I), (I′) or (I″), or a pharmaceutically acceptable salt or solvate thereof.

The invention also provides a method of treating a respiratory disease, such as asthma and rhinitis, especially asthma, in a patient suffering from, or at risk of, said disease, which comprises administering to the patient a therapeutically effective amount of a compound of formula (I), (I′) or (I″), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

The compounds of formula (I), (I′) or (I″), and pharmaceutically acceptable salts and solvates thereof may be used on their own but will generally be administered in the form of a pharmaceutical composition in which the formula (I), (I′) or (I″) compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), (I′) or (I″), or a pharmaceutically acceptable salt or solvate thereof, as hereinbefore defined, in association with a pharmaceutically acceptable adjuvant, diluent or carrier.

The invention further provides a process for the preparation of a pharmaceutical composition of the invention which comprises mixing a compound of formula (I), (I′) or (I″), or a pharmaceutically acceptable salt or solvate thereof, as hereinbefore defined, with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations, or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

The invention further relates to combination therapies wherein a compound of the invention or a pharmaceutically acceptable salts or solvate thereof, or a pharmaceutical composition or formulation comprising a compound of formula (I), (I′) or (I″) is administered concurrently or sequentially with therapy and/or an agent for the treatment of any one of asthma, allergic rhinitis, cancer, COPD, rheumatoid arthritis, psoriasis, inflammatory bowel diseases, osteoarthritis or osteoporosis.

In particular, for the treatment of the inflammatory diseases rheumatoid arthritis, psoriasis, inflammatory bowel disease, COPD, asthma and allergic rhinitis the compounds of the invention may be combined with agents such as TNF-α inhibitors such as anti-TNF monoclonal antibodies (such as Remicade, CDP-870 and D₂E₇ and TNF receptor immunoglobulin molecules (such as Enbrel®), non-selective COX-1/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin), COX-2 inhibitors (such as meloxicam, celecoxib, rofecoxib, valdecoxib and etoricoxib) low dose methotrexate, lefunomide, ciclesonide, hydroxychloroquine, d-penicillamine, auranofin or parenteral or oral gold.

The present invention still further relates to the combination of a compound of the invention together with a leukotriene biosynthesis inhibitor, 5-lipoxygenase (5-LO) inhibitor or 5-lipoxygenase activating protein (FLAP) antagonist such as zileuton, ABT-761, fenleuton, tepoxalin, Abbott-79175, Abbott-85761, N-(5-substituted)-thiophene-2-alkylsulfonamides, 2,6-di-tert-butylphenol hydrazones, methoxytetrahydropyrans such as Zeneca ZD-2138, the compound SB-210661, pyridinyl-substituted 2-cyanonaphthalene compounds such as L-739,010, 2-cyanoquinoline compounds such as L-746,530, indole and quinoline compounds such as MK-591, MK-886, and BAY×1005.

The present invention still further relates to the combination of a compound of the invention together with a receptor antagonist for leukotrienes LTB₄, LTC₄, LTD₄, and LTE₄ selected from the group consisting of the phenothiazin-3-ones such as L-651,392, amidino compounds such as CGS-25019c, benzoxalamines such as ontazolast, benzenecarboximidamides such as BIIL 284/260, and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention together with a PDE4 inhibitor including inhibitors of the isoform PDE4D.

The present invention still further relates to the combination of a compound of the invention together with a antihistaminic H₂ receptor antagonists such as cetirizine, loratadine, desloratadine, fexofenadine, astemizole, azelastine, and chlorpheniramine.

The present invention still further relates to the combination of a compound of the invention together with a gastroprotective H₂ receptor antagonist.

The present invention still further relates to the combination of a compound of the invention together with an α₁.- and α₂.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents such as ipratropium bromide, tiotropium bromide, oxitropium bromide, pirenzepine, and telenzepine.

The present invention still further relates to the combination of a compound of the invention together with a β₁- to β₄-adrenoceptor agonists such as metaproterenol, isoproterenol, isoprenaline, albuterol, salbutamol, formoterol, salmeterol, terbutaline, orciprenaline, bitolterol mesylate, and pirbuterol, or methylxanthanines including theophylline and aminophylline, sodium cromoglycate, or muscarinic receptor (M1, M2, and M3) antagonist.

The present invention still further relates to the combination of a compound of the invention together with an insulin-like growth factor type I (IGF-1) mimetic.

The present invention still further relates to the combination of a compound of the invention together with an inhaled glucocorticoid with reduced systemic side effects, such as prednisone, prednisolone, flunisolide, triamcinolone acetonide, beclomethasone dipropionate, budesonide, fluticasone propionate, and mometasone furoate.

The present invention still further relates to the combination of a compound of the invention together with an inhibitor of matrix metalloproteases (MMPs), i.e., the stromelysins, the collagenases, and the gelatinases, as well as aggrecanase, especially collagenase-1 (MMP-1), collagenase-2 (MMP-8), collagenase-3 (1-13), stromelysin-1 (MMP-3), stromelysin-2 (MMP-10), and stromelysin-3 (MMP-11) and MMP-12.

The present invention still further relates to the combination of a compound of the invention together with other modulators of chemokine receptor function such as CCR1, CCR2, CCR2A, CCR2B, CCR3, CCR4, CCR5, CCR6, CCR7, CCR8, CCR9, CCR10 and CCR11 (for the C—C family), CXCR1, CXCR2, CXCR3, CXCR4 and CXCR5 (for the C—X—C family) and CX₃CR1 for the C—X₃—C family.

The present invention still further relates to the combination of a compound of the invention together with antiviral agents such as Viracept, AZT, aciclovir and famciclovir, and antisepsis compounds such as Valant.

The present invention still further relates to the combination of a compound of the invention together with cardiovascular agents such as calcium channel blockers, lipid lowering agents such as statins, fibrates, beta-blockers, Ace inhibitors, Angiotensin-2 receptor antagonists and platelet aggregation inhibitors.

The present invention still further relates to the combination of a compound of the invention together with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, COX-2 inhibitors, propentofylline or metrifonate.

The present invention still further relates to the combination of a compound of the invention together with (i) tryptase inhibitors, (ii) platelet activating factor (PAF) antagonists, (iii) interleukin converting enzyme (ICE) inhibitors, (iv) IMPDH inhibitors, (v) adhesion molecule inhibitors including VLA-4 antagonists, (vi) cathepsins, (vii) MAP kinase inhibitors, (viii) glucose-6 phosphate dehydrogenase inhibitors, (ix) kinin-B₁- and B₂-receptor antagonists, (x) anti-gout agents, e.g., colchicine, (xi) xanthine oxidase inhibitors, e.g., allopurinol, (xii) uricosuric agents, e.g., probenecid, sulfinpyrazone, and benzbromarone, (xiii) growth hormone secretagogues, (xiv) transforming growth factor (TGFβ), (xv) platelet-derived growth factor (PDGF), (xvi) fibroblast growth factor, e.g., basic fibroblast growth factor (bPGF), (xvii) granulocyte macrophage colony stimulating factor (GM-CSF), (xviii) capsaicin cream, (xix) Tachykinin NK₁ and NK₃ receptor antagonists selected from the group consisting of NKP-608C, SB-233412 (talnetant), and D-4418, (xx) elastase inhibitors selected from the group consisting of UT-77 and ZD-0892, (xxi) TNFα converting enzyme inhibitors (TACE), (xxii) induced nitric oxide synthase inhibitors (iNOS) or (xxiii) chemoattractant receptor-homologous molecule expressed on TH2 cells, (CRTH2 antagonists).

The compounds of the present invention may also be used in combination with osteoporosis agents such as roloxifene, droloxifene, lasofoxifene or fosomax and immunosuppressant agents such as FK-506, rapamycin, cyclosporine, azathioprine, and methotrexate.

The compounds of the invention may also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc and P2X7 receptor antagonists.

The compounds of the invention can also be used in combination with existing therapeutic agents for the treatment of cancer. Suitable agents to be used in combination include:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas), antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®), antitumour antibiotics (for example anthracyclines like adriamycin, bleomycin, doxorubicin, daunomycin, epirubicin, idarubicin, mitomycin-C, dactinomycin and mithramycin), antimitotic agents (for example vinca alkaloids like vincristine, vinblastine, vindesine and vinorelbine and taxoids like taxol and taxotere), and topoisomerase inhibitors (for example epipodophyllotoxins like etoposide and teniposide, amsacrine, topotecan and camptothecin),

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride,

(iii) Agents which inhibit cancer cell invasion (for example metalloproteinase inhibitors like marimastat and inhibitors of urokinase plasminogen activator receptor function),

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family,

(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin),

(vi) vascular damaging agents such as Combretastatin A4 and compounds disclosed in International Patent Applications WO 99/02166, WO00/40529, WO 00/41669, WO01/92224, WO02/04434 and WO02/08213,

(vii) antisense therapies, for example those which are directed to the targets listed above, such as ISIS 2503, an anti-ras antisense,

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy, and

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

General Procedures

¹H NMR and ¹³C NMR were recorded on a Varian Inova 400 MHz, a Bruker Avance DRX 400 or a Varian Mercury-VX 300 MHz instrument. The central peaks of chloroform-d (OH 7.27 ppm), diimethylsulfoxide-d₆ (δ_{H} 2.50 ppm), acetonitrile-d₃ (δ_{H} 1.95 ppm) or methanol-d₄ (δ_{H} 3.31 ppm) were used as internal references.

Column chromatography was carried out using silica gel (0.040-0.063 mm, Merck).

LC-MS Conditions:

Method A: Instrument Agilent 1100, Column: Waters Symmetry 2.1×30 mm, C18 3.5 μm, Mass APCI, Flow rate 0.7 ml/min, Wavelength 220 nm, Solvent A: water+0.1% TFA, solvent B: acetonitrile+0.1% TFA , Gradient 5-95%/B 8 min, 95% B 2 min. retention times (RT) are recorded in minutes.

Method B: Mass Spectrometer—Finnigan TSQ700 with electrospray source operating in positive or negative ion mode. HP1050 system running at 2.0 ml/min, 200 μL/min split to the ESI source with inline HP1050 Single Wavelength UV detector at 254 nm.

Mobile Phase

A) Water 0.1% formic Acid; B) Acetonitrile 0.1% formic Acid

Gradient

| Time | flow | % A | % B |
|---|---|---|---|
| 0.00 | 2.0 | 95 | 5 |
| 1.00 | 2.0 | 95 | 5 |
| 15.00 | 2.0 | 5 | 95 |
| 17.00 | 2.0 | 5 | 95 |
| 18.00 | 2.0 | 95 | 5 |
| 20.00 | 2.0 | 95 | 5 |

Column—Higgins Clipeus C18 5 um 100×3.0 mm

Method C: Mass Spectrometer—Platform LCT with electrospray source operating in positive ion mode. Waters 1525 1c pump running at 1.0 ml/min, ETS PAL autosampler, 100 μl/min split to the ESI source with inline Waters UV2488 Dual Wavelength UV detector at 254 nm and Sedex ELS detection.

Mobile Phase

A) Water 0.1% formic Acid; B) Acetonitrile 0.1% formic Acid

Gradient

| Time | flow | % A | % B |
|---|---|---|---|
| 0.00 | 1.0 | 95 | 5 |
| 1.00 | 1.0 | 95 | 5 |
| 15.00 | 1.0 | 5 | 95 |
| 20.00 | 1.0 | 5 | 95 |
| 22.00 | 1.0 | 95 | 5 |
| 25.00 | 1.0 | 95 | 5 |

Column—Higgins Clipeus C18 5 um 100×3.0 mm

Method D: Mass Spectrometer—Platform LCT with electrospray source operating in positive ion mode. Waters 1525 1c plump running at 2.0 ml/min, HTS PAL autosampler, 200 μL/min split to the ESI source with inline Waters UV2488 Dual Wavelength UV detector at 254 nm and Sedex ELS detection.

Mobile Phase

A) Water 0.1% formic Acid; B) Acetonitrile 0.1% formic Acid

Gradient

| Time | flow | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Column—Waters Atlantis dC18 3 um 4.6×20 mm IS column

Method E: Mass Spectrometer—Platform LC with electrospray source operating in positive and negative ion mode. HP1100 system running at 2.0 ml/min, 200 μL/min split to the ESI source with inline HP1100 DAD detection and SEDEX ELS detection.

Mobile Phase

A) Water 0.1% Formic Acid; B) Acetonitrile 0.1% Formic Acid

Gradient

| Time | flow | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Column—Luna 3u C18(2) 30×4.6 mm

Method F: Mass Spectrometer—Platform ZQ with electrospray source operating in positive and negative ion mode. HP1100 system running at 2.0 ml/min, 200 μL/min split to the ESI source with inline HP1100 DAD detection and SEDEX ELS detection.

Mobile Phase

A) Water 0.1% Formic Acid; B) Acetonitrile 0.1% Formic Acid

Gradient

| Time | flow | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Column—Luna 3u C18(2) 30×4.6 mm

Reverse Phase High Pressure Liquid Chromatography purification was performed using either a Genesis HPLC Column (Ref. 16R10985, 100 mm×22.5 mm) containing C18-7 μm 120A silica; or a Purospher STAR (50 mm×21.2 mm) containing C18 5 μm, Solvent A: water+0.1% TFA, solvent B: acetonitrile+0.1% TFA, Flow: 15 ml/min.

Unless stated otherwise, starting materials were commercially available. All solvents and commercial reagents were of laboratory grade and were used as received.

The following abbreviations are used:

HBTU=O-(Benzotriazol-1-yl)-N,N,N′,N′-tetramethyluronium hexafluorophosphate

DEA=N,N-Diisopropylethylamine

NMP=1-N-Methyl-2-pyrrolidinone

Compounds are named according to ACD naming software (Version ACD/Labs 6.00 (build 6.06/11 Jun. 2002).

### Preparative Procedures

### EXAMPLE 1

### 3-benzoyl-9-(2-ethoxybenzyl)-3,9-diazaspiro[5.5]undecane trifluoroacetate

### a) tert-butyl 9-benzoyl-3,9-diazaspiro[5.5]undecane-3-carboxylate

tert-Butyl 3,9-diazaspiro[5.5]undecane-3-carboxylate (3.44 mmol, 1.00 g), benzoic acid (3.44 mmol, 0.42 g), DIEA (6.88 mmol, 1.18 ml) and HBTU (3.44 mmol, 1.31 g) in NMP (5 ml) were mixed and vigorously stirred for 1 h at room temperature. Water was added and the mixture was extracted with EtOAc. Flash chromatography provided the title compound (0.94 g, 76%).

APCI-MS m/z: 303.2, 359 [MH+]

### b) 3-benzoyl-3,9-diazaspiro[5.5]undecane

tert-butyl 9-benzoyl-3,9-diazaspiro[5.5]undecane-3-carboxylate (3.69 mmol, 1.32 g) was stirred in trifluoroacetic acid (10% in CH₂Cl₂) for 3 h. The solvent was removed and the remaining residue was dissolved in methanol and loaded onto a SCX column. The title compound as a free amine was eluted with ammonia in methanol (0.99 g, >100%).

APCI-MS m/z: 259 [MH+]

### c) 3-benzoyl-9-(2-ethoxybenzyl)-3,9-diazaspiro[5.5]undecane trifluoroacetate

3-benzoyl-3,9-diazaspiro[5.5]undecane (0.031 mmol, 8.0 mg) was dissolved in NMP (300 μl) and acetic acid (60 μl), 2-ethoxybenzaldehyde (0.062 mmol, 8.7 μl) and NaCNBH₃ on resin (0.062 mmol, 15.0 mg) were added. The mixture was shaken for 1 h. The resin was filtered off and the pure title compound was obtained by preparative HPLC (8.0 mg, 66%).

¹H NMR (400 MHz, CDCl₃): δ 11.64 (brs, 1H), 7.53-7.27 (m, 7H), 7.02 (t, 1H), 6.94 (d, 1H), 4.29 (brs, 2H), 4.12 (brd, 2H), 3.8-3.3 (brm, 4H), 3.3-3.1 (brm, 2H), 2.9-2.7 (brm, 2H), 2.1-2.0 (brt, 2H), 1.85-1.80 (brd, 2H), 1.7-1.4 (brm, 4H), 1.44 (brt, 3H).

APCI-MS m/z: 393 [MH+]

The following compounds were prepared according to the general procedure used for example 1.

### 3-benzoyl-9-(2-methoxybenzyl)-3,9-diazaspiro[5.5]undecane trifluoroacetate

¹H NMR (400 MHz, CDCl₃): δ 11.64 (brs, 1H), 7.57 (brs, 1H), 7.46-7.36 (m, 6H), 7.05 (t, 1H), 6.96 (d, 1H), 4.27 (brs, 2H), 3.89 (s, 3H), 3.73 (brs, 2H), 3.5-3.3 (brm, 4H), 2.9-2.7 (brm, 2H), 2.1-2.0 (m, 2H), 1.85-1.80 (brd, 2H), 1.7-1.4 (brm, 4H).

APCI-MS m/z: 379 [MH+]

### 3-(4-chlorobenzoyl)-9-(2-phenoxybenzyl)-3,9-diazaspiro[5.5]undecane trifluoroacetate

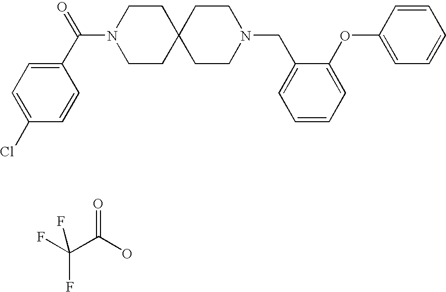

¹H NMR (400 MHz, CD₃OD) δ 7.59 (d, J=7.2 Hz, 1H), 7.51-7.37 (m, 7H), 7.22 (t, J=7.5 Hz, 2H), 7.10 (d, J=7.6 Hz, 2H), 6.88 (d, J=8.4 Hz, 1H), 4.46 (s, 2H), 3.74 (s, 2H), 3.53-3.37 (m, 4H), 3.29-3.16 (m, 2H), 2.05 (d, J=14.6 Hz, 2H), 1.85-1.40 (m, 6H)

APCI-MS m/z: 475/477 (3:1) [MH+]

### 3-benzoyl-9-(3-methoxybenzyl)-3,9-diazaspiro[5.5]undecane trifluoroacetate

LC-MS (Method A) RT: 3.61 min, m/z 380 (MH⁺)

### 3-benzoyl-9-[3-(trifluoromethyl)benzyl]-3,9-diazaspiro[5.5]undecane trifluoroacetate

LC-MS RT: 4.08 min, m/z 417 (MH⁺)

### 3-benzoyl-9-(3,5-dimethoxybenzyl)-3,9-diazaspiro[5.5]undecane trifluoroacetate

LC-MS (Method A) RT: 3.79 min, m/z 409 (MH⁺)

### 3-benzoyl-9-(3-methylbenzyl)-3,9-diazaspiro[5.5]undecane trifluoroacetate

LC-MS (Method A) RT: 3.77 min, m/z 364 (MH⁺)

### 3-benzoyl-9-(3-chlorobenzyl)-3,9-diazaspiro[5.5]undecane trifluoroacetate

LC-MS (Method A) RT: 3.80 min, m/z 383 (MH⁺)

### 3-benzoyl-9-(3-fluoro-2-methylbenzyl)-3,9-diazaspiro[5.5]undecane trifluoroacetate

LC-MS (Method A) RT: 3.77 min, m/z 381 (MH⁺)

### 3-[2-(allyloxy)benzyl]-9-benzoyl-3,9-diazaspiro[5.5]undecane trifluoroacetate

LC-MS (Method A) RT: 4.05 min, m/z 405 (MH⁺)

### 3-benzoyl-9-[3-(trifluoromethoxy)benzyl]-3,9-diazaspiro[5.5]undecane trifluoroacetate

LC-MS (Method A) RT: 4.21 min, m/z 433 (MH⁺)

### 3-benzoyl-9-(2-fluoro-5-methoxybenzyl)-3,9-diazaspiro[5.5]undecane trifluoroacetate

LC-MS (Method A) RT: 3.64 min, m/z 397 (MH⁺)

### 3-benzoyl-9-(4-fluoro-3-methylbenzyl)-3,9-diazaspiro[5.5]undecane trifluoroacetate

LC-MS (Method A) RT: 3.89 min, m/z 381 (MH⁺)

### 3-benzoyl-9-[2-(benzyloxy)benzyl]-3,9-diazaspiro[5.5]undecane trifluoroacetate

LC-MS (Method A) RT: 4.54 min, m/z 455 (MH⁺)

### 3-benzoyl-9-(5-bromo-2-ethoxybenzyl)-3,9-diazaspiro[5.5]undecane trifluoroacetate

LC-MS (Method A) RT: 4.30 min, m/z 471 (MH⁺)

### 3-benzoyl-9-(3-ethoxybenzyl)-3,9-diazaspiro[5.5]undecane trifluoroacetate

LC-MS (Method A) RT: 3.90 min, m/z 393 (MH⁺)

### EXAMPLE 2

### 3-(2-ethoxybenzyl)-9-(4-ethylbenzoyl)-3,9-diazaspiro[5.5]undecane trifluoroacetate

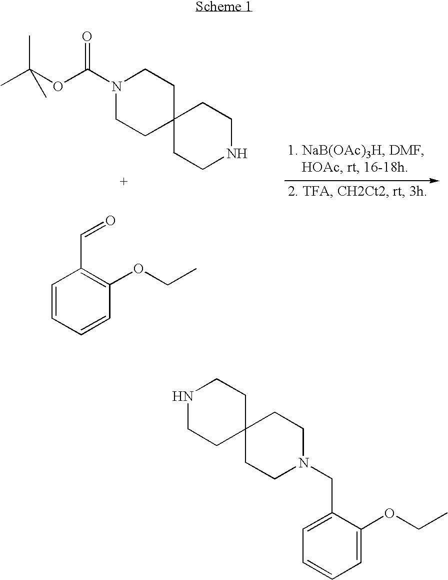

### a) 3-(2-ethoxybenzyl)-3,9-diazaspiro[5.5]undecane

tert-Butyl 3,9-diazaspiro[5.5]undecane-3-carboxylate (0.75 g, 2.9 mmol), 2-ethoxybenzaldehyde (0.646 g, 4.3 mmol) and sodium triacetoxyborohydride (1.23 g, 5.8 mmol) was stirred in DMF (16 ml) and acetic acid (4.5 ml) for 16 h at room temperature. The reaction mixture was diluted with water (20 ml) and the pH was adjusted to 8-9 with saturated Na₂CO₃. The product was extracted with EtOAc, washed with water, dried and the solvent was evaporated. The resulting material was dissolved in methylene chloride (30 ml) and TFA (3 ml) was added. The solution was stirred for 3 h at room temperature. The residue after evaporation was dissolved in MeOH and absorbed onto SCX resin. The product was eluted with 10% ammonia in MeOH and the filtrate was evaporated to give the title compound (664 mg, 79%).

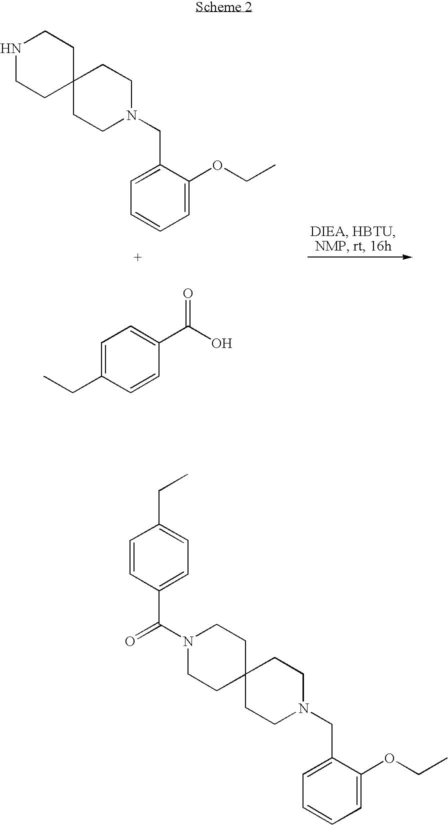

### b) 3-(2-ethoxybenzyl)-9-(4-ethylbenzoyl)-3,9-diazaspiro[5.5]undecane trifluoroacetate

3-(2-ethoxybenzyl)-3,9-diazaspiro[5.5]undecane (1.0 equiv), 4-ethylbensoic acid (1.2 equiv), DIEA (2.3 equiv) and HBTU (1.0 equiv) in NMP (370 μl) were mixed and vigorously stirred for 18 h at room temperature. The pure title compound was obtained by preparative HPLC.

LC-MS (Method A) RT: 4.50 min, m/z 421 (MH⁺)

The following compounds were prepared according to the general procedure used for example 2.

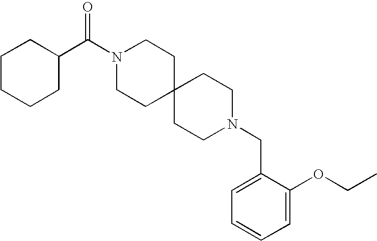

### 3-(cyclohexylcarbonyl)-9-(2-ethoxybenzyl)-3,9-diazaspiro[5.5]undecane

¹H NMR (400 MHz, CD₃OD) δ 7.48 (m, 1H), 7.43 (d, J=6.8 Hz, 1H), 7.12 (d, J=8.7 Hz, 1H), 7.05 (m, 1H), 4.34 (s, 2H), 4.19 (q, J=7.3 Hz, 2H), 3.62-3.12 (m, 8H), 2.63 (m, 1H), 2.00 (d, 2H), 1.83-1.60 (m, 9H), 1.51-1.22 (m, 10H)

APCI-MS m/z: 400 [MH+]

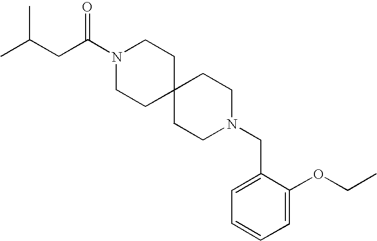

### 3-(2-ethoxybenzyl)-9-(3-methylbutanoyl)-3,9-diazaspiro[5.5]undecane trifluoroacetate

¹H NMR (400 MHz, CD₃OD) δ 7.48 (m, 1H), 7.43 (d, J=7.2 Hz, 1H), 7.13 (d, J=9.1 Hz, 1H), 7.05 (m, 1H), 4.35 (s, 2H), 4.19 (q, J=6.9 Hz, 2H), 3.62-3.12 (m, 8H), 2.28 (m, 2H), 2.10-1.96 (m, 3H), 1.73-1.59 (m, 4H), 1.45 (m, 5H), 0.96 (m, 6H)

APCI-MS m/z: 373 [MH+]

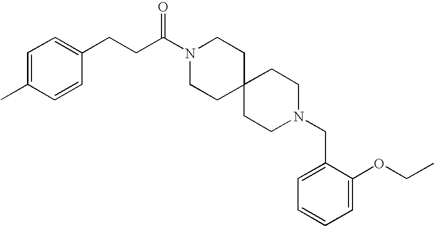

### 3-(2-ethoxybenzyl)-9-[3-(4-methylphenyl)propanoyl]-3,9-diazaspiro[5.5]undecane trifluoroacetate

¹H NMR (400 MHz, CD₃OD) δ 7.52 (t, J=8.2 Hz, 1H), 7.45 (d, J=7.7 Hz, 1H), 7.20-7.03 (m, 6H), 4.36 (s, 2H), 4.22 (q, J=7.1 Hz, 2H), 3.70-3.06 (m, 8H), 2.94-2.88 (m, 2H), 2.73-2.67 (m, 2H), 2.32 (d, J=4.5 Hz, 3H), 1.98-1.89 (m, 2H), 1.68-1.55 (m, 3H), 1.51 (t, J=7.5 Hz, 3H), 1.44-1.37 (m, 2H), 1.22-1.19 (m, 1H)

APCI-MS m/z: 435 [MH+]

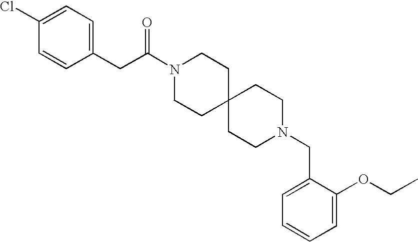

### 3-[(4-chlorophenyl)acetyl]-9-(2-ethoxybenzyl)-3,9-diazaspiro[5.5]undecane trifluoroacetate

¹H NMR (400 MHz, CD₃OD) δ 7.51 (t, 1H), 7.45 (d, 1H), 7.38-7.34 (m, 2H), 7.29-7.26 (m, 2H), 7.15 (d, 1H), 7.08 (t, 1H), 4.36 (d, 2H), 4.22 (q, 2H), 3.81 (d, 2H), 3.65-3.63 (m, 2H), 3.57-3.55 (m, 2H), 3.42-3.17 (m, 6H), 1.98 (d, 2H), 1.69-1.59 (m, 2H), 1.50 (t, 3H), 1.48-1.45 (m, 1H), 1.39-1.34 (m, 1H)

APCI-MS m/z: 441 [MH+]

### 2-(4-chlorobenzoyl)-7-(2-phenoxybenzyl)-2,7-diazaspiro[3.5]nonane trifluoroacetate

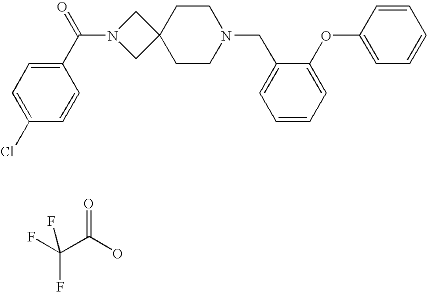

¹H NMR (400 MHz, CD₃OD) δ 7.69-7.63 (m, 2H), 7.62-7.55 (m, 1H), 7.51-7.40 (m, 5H), 7.22 (t, J=7.4 Hz, 2H), 7.14-7.06 (m, 2H), 6.88 (d, J=8.1 Hz, 1H), 4.45 (app d, 2H), 4.25 (s, ½×2H), 4.14 (s, ½×2H), 4.02 (s, ½×2H), 3.92 (s, ½×2H), 3.27-3.06 (m, 2H), 2.27 (d, J=14.4 Hz, 2H), 2.09-1.94 (m, 2H)

APCI-MS m/z: 447/449 (3:1) [MH+]

### 3-[(6-chloropyridin-3-yl)carbonyl]-9-(2-ethoxybenzyl)-3,9-diazaspiro[5.5]undecane bis(trifluoroacetate)

LC-MS (Method A) RT: 3.78 min, m/z 428 (MH⁺)

### (4-{[9-(2-ethoxybenzyl)-3,9-diazaspiro[5.5]undec-3-yl]carbonyl}phenyl)dimethylamine bis(trifluoroacetate)

LC-MS (Method A) RT: 3.32 min, m/z 436 (MH⁺)

### 3-(2-ethoxybenzyl)-9-[2-methoxy-4-(methylthio)benzoyl]-3,9-diazaspiro[5.5]undecane trifluoroacetate

LC-MS (Method A) RT: 4.35 min, m/z 469 (MH⁺)

### 3-(4-butoxybenzoyl)-9-(2-ethoxybenzyl)-3,9-diazaspiro[5.5]undecane trifluoroacetate

LC-MS (Method A) RT: 4.96 min, m/z 465 (MH⁺)

### 1-(4-{[9-(2-ethoxybenzyl)-3,9-diazaspiro[5.5]undec-3-yl]carbonyl}phenyl)ethanone trifluoroacetate

LC-MS (Method A) RT: 3.81 min, m/z 435 (MH⁺)

### 3-(2-ethoxybenzyl)-9-(quinolin-2-ylcarbonyl)-3,9-diazaspiro[5.5]undecane bis(trifluoroacetate)

LC-MS (Method A) RT: 4.00 min, m/z 444 (MH⁺)

### 3-(2-ethoxybenzyl)-9-(3-phenoxybenzoyl)-3,9-diazaspiro[5.5]undecane trifluoroacetate

LC-MS (Method A) RT: 4.89 min, m/z 485 (MH⁺)

### 3-(4-tert-butylbenzoyl)-9-(2-ethoxybenzyl)-3,9-diazaspiro[5.5]undecane trifluoroacetate

LC-MS (Method A) RT: 4.96 min, m/z 449 (MH⁺)

### 4-{[9-(2-ethoxybenzyl)-3,9-diazaspiro[5.5]undec-3-yl]carbonyl}benzonitrile trifluoroacetate

LC-MS (Method A) RT: 3.90 min, m/z 418 (MH⁺)

### 3-(2-ethoxybenzyl)-9-(6-methoxy-2-naphthoyl)-3,9-diazaspiro[5.5]undecane trifluoroacetate

LC-MS (Method A) RT: 4.56 min, m/z 473 (MH⁺)

### 3-(2,3-dichlorobenzoyl)-9-(2-ethoxybenzyl)-3,9-diazaspiro[5.5]undecane trifluoroacetate

LC-MS (Method A) RT: 4.46 min, m/z 461 (MH⁺)

### 3-(2-ethoxybenzyl)-9-(3-methoxybenzoyl)-3,9-diazaspiro[5.5]undecane trifluoroacetate

LC-MS (Method A) RT: 4.04 min, m/z 423 (MH⁺)

### 3-(2,3-dimethylbenzoyl)-9-(2-ethoxybenzyl)-3,9-diazaspiro[5.5]undecane trifluoroacetate

LC-MS (Method A) RT: 4.29 min, m/z 421 (MH⁺)

### 3-(4-chlorobenzoyl)-9-(2-ethoxybenzyl)-3,9-diazaspiro[5.5]undecane trifluoroacetate

LC-LC-MS (Method A) RT: 4.34 min, m/z 427 (MH⁺)

### 3-(2-ethoxybenzyl)-9-(4-methylbenzoyl)-3,9-diazaspiro[5.5]undecane trifluoroacetate

LC-LC-MS (Method A) RT: 4.21 min, m/z 407 (MH⁺)

### 3-(3,4-dichlorobenzoyl)-9-(2-ethoxybenzyl)-3,9-diazaspiro[5.5]undecane trifluoroacetate

LC-MS (Method A) RT: 4.69 min, m/z 461 (MH⁺)

### 3-(3,4-dimethoxybenzoyl)-9-(2-ethoxybenzyl)-3,9-diazaspiro[5.5]undecane trifluoroacetate

LC-MS (Method A) RT: 3.82 min, m/z 453 (MH⁺)

### 3-(2,4-dichlorobenzoyl)-9-(2-ethoxybenzyl)-3,9-diazaspiro[5.5]undecane trifluoroacetate

LC-MS (Method A) RT: 4.55 min, m/z 461 (MH⁺)

### 3-(2-ethoxybenzyl)-9-(4-isopropoxybenzoyl)-3,9-diazaspiro[5.5]undecane trifluoroacetate

LC-MS (Method A) RT: 4.51 min, m/z 451 (MH⁺)

### 3-(2-ethoxybenzyl)-9-(4-phenoxybenzoyl)-3,9-diazaspiro[5.5]undecane trifluoroacetate

LC-MS (Method A) RT: 4.90 min, m/z 485 (MH⁺)

### 3-(2-ethoxybenzyl)-9-(2-naphthoyl)-3,9-diazaspiro[5.5]undecane trifluoroacetate

LC-MS (Method A) RT: 4.53 min, m/z 443 (MH⁺)

### 3-(2,3-dimethoxybenzoyl)-9-(2-ethoxybenzyl)-3,9-diazaspiro[5.5]undecane trifluoroacetate

LC-MS (Method A) RT: 3.97 min, m/z 453 (MH⁺)

### 3-(2-ethoxybenzyl)-9-(1-naphthoyl)-3,9-diazaspiro[5.5]undecane trifluoroacetate

LC-MS (Method A) RT: 4.42 min, m/z 443 (MH⁺)

### (3-{[9-(2-ethoxybenzyl)-3,9-diazaspiro[5.5]undec-3-yl]carbonyl}phenyl)dimethylamine bis(trifluoroacetate)

LC-MS (Method A) RT: 3.22 min, m/z 436 (MH⁺)

### 3-(2-ethoxybenzyl)-9-[3-(methylsulfonyl)benzoyl]-3,9-diazaspiro[5.5]undecane trifluoroacetate

LC-MS (Method A) RT: 3.66 min, m/z 471 (MH⁺)

### 3-(2-ethoxybenzyl)-9-(4-methoxybenzoyl)-3,9-diazaspiro[5.5]undecane trifluoroacetate

LC-MS (Method A) RT: 4.02 min, m/z 423 (MH⁺)

### (4-{[9-(2-ethoxybenzyl)-3,9-diazaspiro[5.5]undec-3-yl]carbonyl}phenyl)diethylamine bis(trifluoroacetate)

LC-MS (Method A) RT: 3.24 min, m/z 464 (MH⁺)

### 3-(2-ethoxybenzyl)-9-(4-propylbenzoyl)-3,9-diazaspiro[5.5]undecane trifluoroacetate

LC-MS (Method A) RT: 4.81 min, m/z 435 (MH⁺)

### 3-(2-chloroisonicotinoyl)-9-(2-ethoxybenzyl)-3,9-diazaspiro[5.5]undecane bis(trifluoroacetate)

LC-MS (Method A) RT: 3.74 min, m/z 428 (MH⁺)

### 3-(2-ethoxybenzyl)-9-[3-(trifluoromethyl)benzoyl]-3,9-diazaspiro[5.5]undecane trifluoroacetate

LC-MS RT: 4.56 min, m/z 461 (MH⁺)

### 3-(2-ethoxybenzyl)-9-[4-(trifluoromethyl)benzoyl]-3,9-diazaspiro[5.5]undecane trifluoroacetate

LC-MS (Method A) RT: 4.60 min, m/z 461 (MH⁺)

### 3-(2-ethoxybenzyl)-9-(quinolin-4-ylcarbonyl)-3,9-diazaspiro[5.5]undecane bis(trifluoroacetate)

LC-MS (Method A) RT: 3.23 min, m/z 444 (MH⁺)

### 3-(3-chloro-2-methylbenzoyl)-9-(2-ethoxybenzyl)-3,9-diazaspiro[5.5]undecane trifluoroacetate

LC-MS (Method A) RT: 4.46 min, m/z 441 (MH⁺)

### 3-[2-(benzyloxy)benzyl]-9-[(6-chloropyridin-3-yl)carbonyl]-3,9-diazaspiro[5.5]undecane bis(trifluoroacetate)

LC-MS (Method A) RT: 4.43 min, m/z 490 (MH⁺)

### [4-({9-[2-(benzyloxy)benzyl]-3,9-diazaspiro[5.5]undec-3-yl}carbonyl)phenyl]dimethylamine bis(trifluoroacetate)

LC-MS (Method A) RT: 3.97 min, m/z 498 (MH⁺).

### 3-[2-(benzyloxy)benzyl]-9-[2-methoxy-4-(methylthio)benzoyl]-3,9-diazaspiro[5.5]undecane trifluoroacetate

LC-MS (Method A) RT: 4.88 min, m/z 531 (MH⁺)

### 1-[4-({9-[2-(benzyloxy)benzyl]-3,9-diazaspiro[5.5]undec-3-yl}carbonyl)phenyl]ethanone trifluoroacetate

LC-MS (Method A) RT: 4.42 min, m/z 497 (MH⁺)

### 3-[2-(benzyloxy)benzyl]-9-(4-ethylbenzoyl)-3,9-diazaspiro[5.5]undecane trifluoroacetate

LC-MS (Method A) RT: 5.01 min, m/z 483 (MH⁺)

### 3-[2-(benzyloxy)benzyl]-9-(quinolin-2-ylcarbonyl)-3,9-diazaspiro[5.5]undecane bis(trifluoroacetate)

LC-MS (Method A) RT: 4.58 min, m/z 506 (MH⁺)

### 3-[2-(benzyloxy)benzyl]-9-(4-chloro-2-methoxybenzoyl)-3,9-diazaspiro[5.5]undecane trifluoroacetate

LC-MS (Method A) RT: 4.88 min, m/z 519 (MH⁺)

### 3-({9-[2-(benzyloxy)benzyl]-3,9-diazaspiro[5.5]undec-3-yl}carbonyl)benzonitrile trifluoroacetate

LC-MS (Method A) RT: 4.51 min, m/z 480 (MH⁺)

### 3-[2-(benzyloxy)benzyl]-9-(4-tert-butylbenzoyl)-3,9-diazaspiro[5.5]undecane trifluoroacetate

LC-MS (Method A) RT: 5.41 min, m/z 511 (MH⁺)

### 4-({9-[2-(benzyloxy)benzyl]-3,9-diazaspiro[5.5]undec-3-yl}carbonyl)benzonitrile trifluoroacetate

LC-MS (Method A) RT: 4.52 min, m/z 480 (MH⁺)

### 3-[2-(benzyloxy)benzyl]-9-(4-morpholin-4-ylbenzoyl)-3,9-diazaspiro[5.5]undecane bis(trifluoroacetate)

LC-MS (Method A) RT: 7.18 min, m/z 540 (MH⁺)

### 3-[2-(benzyloxy)benzyl]-9-(2,3-dichlorobenzoyl)-3,9-diazaspiro[5.5]undecane trifluoroacetate

LC-MS (Method A) RT: 4.99 min, m/z 523 (MH⁺)

### 3-[2-(benzyloxy)benzyl]-9-(3-methoxybenzoyl)-3,9-diazaspiro[5.5]undecane trifluoroacetate

LC-MS (Method A) RT: 4.61 min, m/z 485 (MH⁺)

### 3-[2-(benzyloxy)benzyl]-9-(2,3-dimethylbenzoyl)-3,9-diazaspiro[5.5]undecane trifluoroacetate

LC-MS (Method A) RT: 4.84 min, m/z 483 (MH⁺)

### 3-[2-(benzyloxy)benzyl]-9-(4-chlorobenzoyl)-3,9-diazaspiro[5.5]undecane trifluoroacetate

LC-MS (Method A) RT: 4.88 min, m/z 489 (MH⁺)

### 3-[2-(benzyloxy)benzyl]-9-(4-methylbenzoyl)-3,9-diazaspiro[5.5]undecane trifluoroacetate

LC-MS (Method A) RT: 4.77 min, m/z 469 (MH⁺)

### 3-[2-(benzyloxy)benzyl]-9-(3,4-dimethoxybenzoyl)-3,9-diazaspiro[5.5]undecane trifluoroacetate

LC-MS (Method A) RT: 4.41 min, m/z 515 (MH⁺)

### 3-[2-(benzyloxy)benzyl]-9-(4-isopropoxybenzoyl)-3,9-diazaspiro[5.5]undecane trifluoroacetate

LC-MS (Method A) RT: 5.01 min, m/z 513 (MH⁺)

### 3-[2-(benzyloxy)benzyl]-9-(4-phenoxybenzoyl)-3,9-diazaspiro[5.5]undecane trifluoroacetate

LC-MS (Method A) RT: 5.35 min, m/z 547 (MH⁺)

### 3-[2-(benzyloxy)benzyl]-9-(2-naphthoyl)-3,9-diazaspiro[5.5]undecane trifluoroacetate

LC-MS (Method A) RT: 5.03 min, m/z 505 (MH⁺)

### 3-[2-(benzyloxy)benzyl]-9-(2-chlorobenzoyl)-3,9-diazaspiro[5.5]undecane trifluoroacetate

LC-MS (Method A) RT: 4.68 min, m/z 489 (MH⁺)

### 3-[2-(benzyloxy)benzyl]-9-(2,3-dimethoxybenzoyl)-3,9-diazaspiro[5.5]undecane trifluoroacetate

LC-MS (Method A) RT: 4.57 min, m/z 515 (MH⁺)

### 3-[2-(benzyloxy)benzyl]-9-(1-naphthoyl)-3,9-diazaspiro[5.5]undecane trifluoroacetate

LC-MS (Method A) RT: 4.92 min, m/z 505 (MH⁺)

### [3-({9-[2-(benzyloxy)benzyl]-3,9-diazaspiro[5.5]undec-3-yl}carbonyl)phenyl]dimethylamine bis(trifluoroacetate)

LC-MS (Method A) RT: 3.85 min, m/z 498 (MH⁺)

### 3-[2-(benzyloxy)benzyl]-9-(4-methoxybenzoyl)-3,9-diazaspiro[5.5]undecane trifluoroacetate

LC-MS (Method A) RT: 4.60 min, m/z 485 (MH⁺)

### [4-({9-[2-(benzyloxy)benzyl]-3,9-diazaspiro[5.5]undec-3-yl}carbonyl)phenyl]-diethylamine bis(trifluoroacetate)

LC-MS (Method A) RT: 3.83 min, m/z 526 (MH⁺)

### 3-[2-(benzyloxy)benzyl]-9-(2-chloroisonicotinoyl)-3,9-diazaspiro[5.5]undecane bis(trifluoroacetate)

LC-MS (Method A) RT: 4.39 min, m/z 490 (MH⁺)

### 3-[2-(benzyloxy)benzyl]-9-[3-(trifluoromethyl)benzoyl]-3,9-diazaspiro[5.5]undecane trifluoroacetate

LC-MS (Method A) RT: 5.07 min, m/z 523 (MH⁺)

### 3-[2-(benzyloxy)benzyl]-9-[4-(trifluoromethyl)benzoyl]-3,9-diazaspiro[5.5]undecane trifluoroacetate

LC-MS (Method A) RT: 5.10 min, m/z 523 (MH⁺)

### 3-[2-(benzyloxy)benzyl]-9-(quinolin ylcarbonyl)-3,9-diazaspiro[5.5]undecane bis(trifluoroacetate)

LC-MS (Method A) RT: 3.84 min, m/z 506 (MH⁺)

### EXAMPLE: 3

### 3-benzoyl-9-(2-propoxybenzyl)-3,9-diazaspiro[5.5]undecane trifluoroacetate

### a) 2-propoxybenzaldehyde

To a solution of salicylaldehyde (0.82 mmol, 87 μl) in DMF (250 μl) NaH (60%, 0.85 mmol, 34 mg) was added. 1-bromopropane (0.85 mmol, 94 μl) was added dropwise and the mixture was stirred for 4 h. The mixture was partitioned between water and EtOAc and the organic layer washed and evaporated leaving the title compound (89 mg, 66%) with a purity of 80%.

APCI-MS m/z: 165 [MH+]

### b) 3-benzoyl-9-(2-propoxybenzyl)-3,9-diazaspiro[5.5]undecane trifluoroacetate

3-benzoyl-3,9-diazaspiro[5.5]undecane (0.062 mmol, 16 mg) was dissolved in NMP (400 μl) and acetic acid (200 μl), 2-propoxybenzaldehyde (0.124 mmol) and NaCNBH₃ on resin (0.124 mmol, 30 mg) were added. The mixture was shaken for 1 h. The resin was filtered off and the pure title compound was obtained by preparative HPLC (8 mg, 32%).

¹H NMR (400 MHz, CDCl₃): δ 11.64 (brs, 1H), 7.53-7.36 (m, 7H), 7.04 (t, 1H), 6.96 (d, 1H), 4.31 (brs, 2H), 4.10 (brd, 2H), 3.8-3.1 (brm, 6H), 2.9-2.7 (brm, 2H), 2.1-2.0 (brt, 2H), 1.90-1.80 (brd, 4H), 1.7-1.4 (brm, 4H), 1.05 (brt, 3H).

APCI-MS m/z: 407 [MH+]

The following compounds were prepared according to the general procedure used for example 3.

### 3-benzoyl-9-(2-isobutoxybenzyl)-3,9-diazaspiro[5.5]undecane trifluoroacetate

¹H NMR (400 MHz, CDCl₃): δ 11.59 (brs, 1H), 7.55-7.35 (m, 7H), 7.04 (t, 1H), 6.94 (d, 1H), 4.30 (brs, 2H), 3.8-2.7 (brm, 10H), 2.2-2.0 (brm, 3H), 1.82 (brd, 2H), 1.7-1.4 (brm, 4H), 1.06 (brd, 6H).

APCI-MS m/z: 421 [MH+]

### 2-(4-chlorobenzoyl)-7-(2-isobutoxybenzyl)-2,7-diazaspiro[3.5]nonane trifluoroacetate

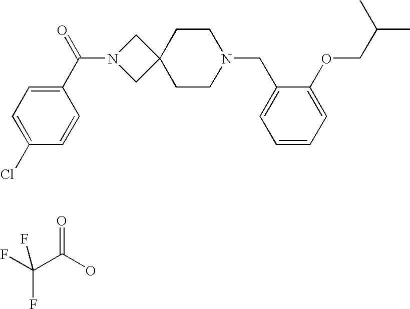

¹H NMR (400 MHz, CD₃OD) δ 7.66 (t, J=8.3 Hz, 2H), 7.53-7.38 (m, 4H), 7.12 (d, J=8.3 Hz, 1H), 7.05 (t, J=7.5 Hz, 1H), 4.33 (app d, 2H), 4.24 (s, ½×2H), 4.12 (s, ½×2H), 4.03 (s, ½×2H), 3.91 (s, ½×2H), 3.89 (t, J=6.2 Hz, 2H), 3.22-3.00 (m, 2H), 2.16 (quintet, J=6.8 Hz, 1H), 2.04-1.91 (m, 2H), 1.08 (app t, 6H)

APCI-MS m/z: 427/429 (3:1) [MH+]

### EXAMPLE: 4

### 3-benzoyl-9-[2-(tetrahydrofuran-2-ylmethoxy)benzyl]-3,9-diazaspiro[5.5]undecane trifluoroacetate

### a) 2-(tetrahydrofuran-2-ylmethoxy)benzaldehyde

To a solution of salicylaldehyde (0.82 mmol, 87 μl) in DMF (250 μl) NaH (60%, 0.85 mmol, 34 mg) was added. 2-(bromomethyl)tetrahydrofuran (1.04 mmol, 118 μl) was added dropwise and the mixture was stirred for 4 h at 90° C. The mixture was partitioned between water and EtOAc and the organic layer washed and evaporated leaving the title compound.

APCI-MS m/z: 207 [MH+]

### b) 3-benzoyl-9-[2-(tetrahydrofuran-2-ylmethoxy)benzyl]-3,9-diazaspiro[5,5]undecane trifluoroacetate

3-benzoyl-3,9-diazaspiro[5.5]undecane (0.062 mmol, 16 mg) was dissolved in NMP (400 μl) and acetic acid (200 μl), 2-(tetrahydrofuran-2-ylmethoxy)benzaldehyde (0.124 mmol) and NaCNBH₃ on resin (0.124 mmol, 30 mg) were added. The mixture was shaken for 1 h. The resin was filtered off and the pure title compound was obtained by preparative HPLC.

¹H NMR (400 MHz, CDCl₃): δ 11.33 (brs, 1H), 7.47-7.35 (m, 7H), 7.04 (t, 1H), 6.92 (d, 1H), 5.09 (brs, 2H), 4.32 (brs, 2H), 4.10 (brd, 1H), 3.9-3.3 (m, 9H), 2.99 (brs, 2H), 2.2-1.4 (m, 11H).

APCI-MS m/z: 449 [MH+]

The following compounds were prepared according to the general procedure used for example 4.

### 3-benzoyl-9-[2-(tetrahydro-2H-pyran-2-ylmethoxy)benzyl]-3,9-diazaspiro[5.5]undecane trifluoroacetate

¹H NMR (400 MHz, CDCl₃): δ 11.36 (brs, 1H), 7.56-7.35 (m, 7H), 7.05 (t, 1H), 6.92 (d, 1H), 4.35-4.27 (m, 2H), 3.99-3.92 (m, 3H), 3.8-3.3 (m, 9H), 2.96 (brs, 2H), 2.2-1.4 (m, 13H).

APCI-MS m/z: 463 [MH+]

### 3-benzoyl-9-{2-[(3,5-dimethylisoxazol-4-yl)methoxy]benzyl}-3,9-diazaspiro[5.5]undecane trifluoroacetate

¹H NMR (400 MHz, CDCl₃): δ 11.85 (brs, 1H), 7.66 (m, 1H), 7.48-7.35 (m, 6H), 7.12 (t, 1H), 7.05 (d, 1H), 4.87 (brs, 2H), 4.18 (brs, 2H), 3.8-2.6 (brm, 8H), 2.42 (brs, 3H), 2.28 (brs, 3H), 2.10 (brt, 2H), 1.76 (brd, 2H), 1.6-1.4 (brm, 4H).

APCI-MS m/z: 474 [MH+]

### {2-[(9-benzoyl-3,9-diazaspiro[5.5]undec-3-yl)methyl]phenoxy}acetonitrile trifluoroacetate

¹H NMR (400 MHz, CDCl₃): δ 12.27 (brs, 1H), 7.83 (d, 1H), 7.53 (m, 1H), 7.45-7.35 (m, 5H), 7.21 (t, 1H), 7.06 (d, 1H), 4.99 (brs, 2H), 4.24 (brs, 2H), 3.8-3.6 (brm, 2H), 3.4-3.2 (m, 4H), 2.9-2.7 (brm, 2H), 2.35 (brt, 2H), 1.76 (brd, 2H), 1.6-1.4 (brm, 4H).

APCI-MS m/z: 404 [MH+]

### EXAMPLE: 5

### 3-(2-propoxybenzyl)-9-(pyridin-3-ylcarbonyl)-3,9-diazaspiro[5.5]undecane bis(trifluoroacetate)

### a) tert-butyl 9-(pyridin-3-ylcarbonyl)-3,9-diazaspiro[5.5]undecane-3-carboxylate

tert-Butyl 3,9-diazaspiro[5.5]undecane-3-carboxylate (1.72 mmol, 500 mg), nicotinic acid (1.72 mmol, 212 mg), DIEA (3.44 mmol, 589 μl) and HBTU (1.72 mmol, 652 mg) in NMP (2.5 ml) were mixed and vigorously stirred for 1 h at room temperature. Water was added and the mixture was extracted with EtOAc. Flash chromatography provided the title compound (476 g, 77%).

APCI-MS m/z: 304 [MH+]

### b) 3-(pyridin-3-ylcarbonyl)-3,9-diazaspiro[5.5]undecane

tert-butyl 9-(pyridin-3-ylcarbonyl)-3,9-diazaspiro[5.5]undecane-3-carboxylate (1.32 mmol, 476 mg) was stirred in trifluoroacetic acid (10% in CH₂Cl₂) for 3 h. The solvent was removed and the remaining residue was dissolved in methanol and loaded onto a SCX column. The title compound as a free amine was eluted with ammonia in methanol.

APCI-MS m/z: 260 [MH+]

### c) 3-(2-propoxybenzyl)-9-(pyridin-3-ylcarbonyl)-3,9-diazaspiro[5.5]undecane bis(trifluoroacetate)

3-(pyridin-3-ylcarbonyl)-3,9-diazaspiro[5.5]undecane (0.062 mmol, 16.0 mg) was dissolved in NMP (400 μl) and acetic acid (200 μl), 2-propoxybenzaldehyde (0.124 mmol) and NaCNBH₃ on resin (0.124 mmol, 30.0 mg) were added. The mixture was shaken for 1 h. The resin was filtered off and the pure title compound was obtained by preparative HPLC.

¹H NMR (400 MHz, CDCl₃): δ 11.47 (brs, 1H), 8.83-8.77 (m, 2H), 8.2-8.1 (m, 1H), 7.72 (s, 1H), 7.48 (d, 1H), 7.43 (t, 1H), 7.01 (t, 1H), 6.96 (d, 1H), 4.29 (s, 2H), 4.00 (brs, 2H), 3.74 (brs, 2H), 3.50-3.40 (brm, 4H), 2.84 (brs, 2H), 2.09 (brt, 2H), 1.90-1.79 (m, 4H), 1.7-1.4 (brm, 4H), 1.06 (brs, 3H).

APCI-MS m/z: 408 [MH+]

### EXAMPLE: 6

### 2-[(4-chlorophenyl)sulfonyl]-7-(2-phenoxybenzyl)-2,7-diazaspiro[3.5]nonane trifluoroacetate

7-(2-phenoxybenzyl)-2,7-diazaspiro[3.5]nonane dihydrochloride (0.11 mmol, 42 mg), 4-chlorobenzenesulfonyl chloride (0.13 mmol, 28 mg) and DIEA (0.33 mmol, 56 μl) in DMF (500 μl) were mixed and vigorously stirred overnight at room temperature. Water and CH₃CN (1:1, 1 ml) was added and the pure title compound was obtained by preparative HPLC (47 mg, 72%).

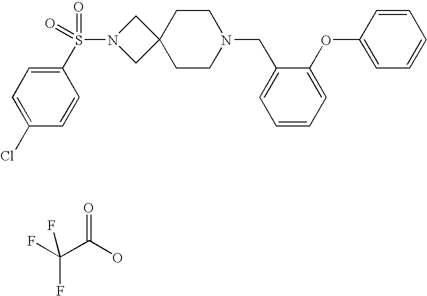

¹H NMR (400 MHz, CD₃OD) δ 7.84 (d, J=9.8 Hz, 2H), 7.69 (d, J=9.1 Hz, 2H), 7.54 (dd, J=7.6, 1.5 Hz, 1H), 7.46-7.39 (m, 3H), 7.26-7.17 (m, 2H), 7.07 (d, J=7.8 Hz, 2H), 6.86 (d, J=8.3 Hz, 1H), 4.38 (s, 2H), 3.67 (s, 2H), 3.56 (s, 2H), 3.12-2.98 (m, 2H), 1.96-1.77 (m, 4H)

APCI-MS m/z: 483/485 (3:1) [MH+]

### EXAMPLE: 7

### 3-(2-isobutoxybenzyl)-9-(pyridin-4-ylcarbonyl)-3,9-diazaspiro[5.5]undecane dihydrochloride

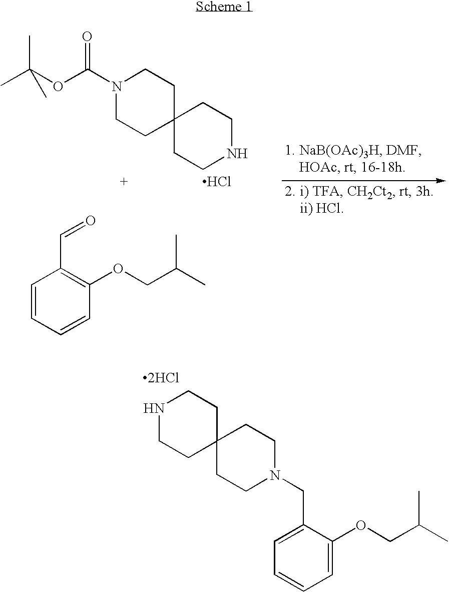

### a) 3-(2-isobutoxybenzyl)-3,9-diazaspiro[5.5]undecane dihydrochloride

A mixture of tert-butyl 3,9-diazaspiro[5.5]undecane-3-carboxylate hydrochloride (1.0 g, 3.44 mmol), 2-isobutoxybenzaldehyde (0.612 g, 3.44 mmol), triethylamine (0.718 ml, 5.16 mmol), sodium triacetoxyborohydride (1.02 g, 4.81 mmol) and dichloroethane (25 ml) was stirred at room temperature overnight. The reaction mixture was concentrated, then partitioned between ethyl acetate and saturated sodium hydrogen carbonate solution. The organic layer was isolated and evaporated to dryness to provide an oil. The oil was dissolved in dichloromethane (25 ml), and then trifluoroacetic acid (5 ml) was added. After stirring for 3 hours the reaction mixture was concentrated to give an orange oil which was dissolved in ethyl acetate and washed with 1M hydrochloric acid (3×). The combined aqueous layers were concentrated, then azeotroped with toluene, and triturated with diethyl ether to provide the title compound (1.2 g, 3.09 mmol) as an off-white solid.

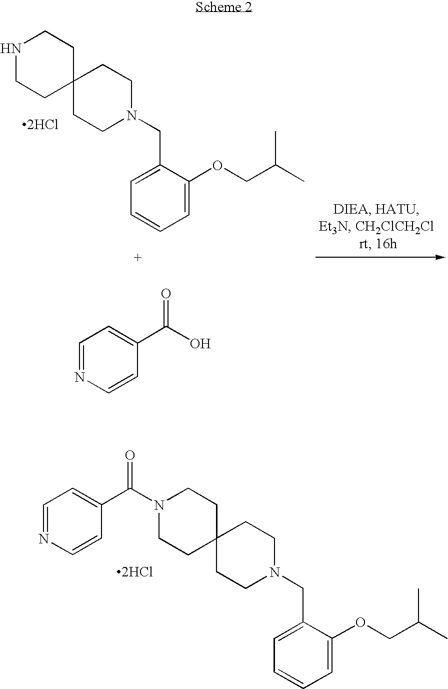

### b) 3-(2-isobutoxybenzyl)-9-isonicotinoyl-3,9-diazaspiro[5.5]undecane dihydrochloride

To a solution of 3-(2-isobutoxybenzyl)-3,9-diazaspiro[5.5]undecane dihydrochloride (42 mg, 0.11 mmol, 1 equiv), isonicotinic acid (18 mg, 0.14 mmol, 1.2 equiv) and diisopropylethylamine (86 μl, 0.50 mmol, 4.5 equiv) in dry dichloromethane (4 ml), was added O-(7-azabenzotriazol-1-yl)-N,N,N′N′-tetramethyluronium hexafluorophosphate (46 mg, 0.12 mmol, 1.05 equiv). The reaction mixture was stirred at room temperature overnight, then concentrated, and subjected to chromatography using an Isolute® flash NH₂ cartridge and a mixture of ethyl acetate and cyclohexane (gradient 10:90 to 50:50, v/v) as eluent to give an oil. The oil was subsequently triturated with 1.25M hydrochloric acid in methanol solution to provide an off-white solid, which was filtered, then dried under vacuum to obtain the title compound (32 mg, 59%) as a white solid.

¹H NMR (400 MHz, CD₃OD with NaOD added): δ 8.65 (m, 2H), 7.43 (m, 2H), 7.28 (dd, 1H), 7.24 (ddd, 1H), 6.93 (dd, 1H), 6.90 (td, 1H), 3.76 (d, 2H), 3.72 (m, 2H), 3.62 (s, 2H), 3.32 (m, 2H), 2.53 (br m, 4H), 2.09 (m, 1H), 1.60 (m, 6H), 1.45 (m, 2H), 1.06 (d, 6H).

LCMS (Method C): R_{T}=5.98 minutes; 422 (M+H)⁺.

The following compounds were prepared according to the general procedure used for example 7.

| | LCMS | Retention | Mass Ion/ |
|---|---|---|---|
| Compound | Method | time/min | MH⁺ |
| 3-(4-chlorobenzoyl)-9-[2-(pyridin-2-ylmethoxy)benzyl]- | B | 5.05 | 490/492 |
| 3,9-diazaspiro[5.5]undecane | | | |
| 3-(4-chlorobenzoyl)-9-[3-(pyridin-4-ylmethoxy)benzyl]- | B | 3.81 | 490/492 |
| 3,9-diazaspiro[5.5]undecane | | | |
| 3-{[9-(2-isobutoxybenzyl)-3,9-diazaspiro[5.5]undec-3- | B | 5.34 | 446 |
| yl]carbonyl}benzonitrile | | | |
| 3-(2-isobutoxybenzyl)-9-(pyrazin-2-ylcarbonyl)-3,9- | C | 6.46 | 423 |
| diazaspiro[5.5]undecane | | | |
| 3-(2-isobutoxybenzyl)-9-(pyrimidin-2-ylcarbonyl)-3,9- | C | 6.34 | 423 |
| diazaspiro[5.5]undecane | | | |
| 3-(2-isobutoxybenzyl)-9-(pyrimidin-4-ylcarbonyl)-3,9- | C | 6.39 | 423 |
| diazaspiro[5.5]undecane | | | |
| 3-(2-isobutoxybenzyl)-9-(pyrimidin-5-ylcarbonyl)-3,9- | C | 6.35 | 423 |
| diazaspiro[5.5]undecane | | | |
| 3-(4-chlorobenzoyl)-9-[(6-isobutoxypyridin-2-yl)methyl]- | C | 7.77 | 456/458 |
| 3,9-diazaspiro[5.5]undecane | | | |
| 2-(4-chlorobenzoyl)-7-(3-phenoxybenzyl)-2,7- | C | 7.68 | 447/449 |
| diazaspiro[3.5]nonane | | | |
| 2-benzoyl-7-(3-phenoxybenzyl)-2,7- | C | 7.23 | 413 |
| diazaspiro[3.5]nonane | | | |
| 3-(2-isobutoxybenzyl)-9-(pyridazin-3-ylcarbonyl)-3,9- | C | 6.13 | 423 |
| diazaspiro[5.5]undecane | | | |
| 3-(2-isobutoxybenzyl)-9-(pyridazin-4-ylcarbonyl)-3,9- | C | 6.29 | 423 |
| diazaspiro[5.5]undecane | | | |
| 3-(2-isobutoxybenzyl)-9-(pyridin-2-ylcarbonyl)-3,9- | C | 6.65 | 422 |
| diazaspiro[5.5]undecane | | | |
| 3-(2-isobutoxybenzyl)-9-(pyridin-3-ylcarbonyl)-3,9- | C | 6.29 | 422 |
| diazaspiro[5.5]undecane | | | |
| 3-(4-chlorobenzoyl)-9-[3-(pyridin-2-ylmethoxy)benzyl]- | C | 6.45 | 490/492 |
| 3,9-diazaspiro[5.5]undecane | | | |
| 3-(4-chlorobenzoyl)-9-[3-(pyridin-3-ylmethoxy)benzyl]- | C | 5.7 | 490/492 |
| 3,9-diazaspiro[5.5]undecane | | | |
| 3-(3-furoyl)-9-(2-isobutoxybenzyl)-3,9- | C | 7.09 | 411 |
| diazaspiro[5.5]undecane | | | |
| 3-(2-isobutoxybenzyl)-9-(3-thienylcarbonyl)-3,9- | C | 7.15 | 427 |
| diazaspiro[5.5]undecane | | | |
| 3-(4-chlorobenzoyl)-9-(2-isobutoxybenzyl)-3,9- | B | 5.75 | 455/457 |
| diazaspiro[5.5]undecane | | | |
| 3-benzoyl-9-(2-isobutoxybenzyl)-3,9- | B | 5.54 | 421 |
| diazaspiro[5.5]undecane | | | |
| 2-(3-furoyl)-8-(2-isobutoxybenzyl)-2,8- | B | 5.07 | 397 |
| diazaspiro[4.5]decane | | | |
| 2-{[8-(2-isobutoxybenzyl)-2,8-diazaspiro[4.5]dec-2- | B | 5.5 | 458 |
| yl]carbonyl}quinoline | | | |
| 2-{[2-(2-isobutoxybenzyl)-2,8-diazaspiro[4.5]dec-8- | B | 4.92 | 458 |
| yl]carbonyl}quinoline | | | |
| 8-(2-isobutoxybenzyl)-2-(pyridin-2-ylacetyl)-2,8- | B | 3.66 | 422 |
| diazaspiro[4.5]decane | | | |
| 2-(4-chlorobenzoyl)-7-(2-isobutoxybenzyl)-2,7- | C | 7.81 | 427/429 |
| diazaspiro[3.5]nonane | | | |
| 2-(4-chlorobenzoyl)-7-(2-phenoxybenzyl)-2,7- | C | 7.73 | 447/449 |
| diazaspiro[3.5]nonane | | | |
| 3-[(5-chloro-2-thienyl)carbonyl]-9-(2-isobutoxybenzyl)- | C | 8.03 | 461/463 |
| 3,9-diazaspiro[5.5]undecane | | | |
| 3-(2-isobutoxybenzyl)-9-(1H-pyrrol-2-ylcarbonyl)-3,9- | C | 7.2 | 410 |
| diazaspiro[5.5]undecane | | | |
| 3-(2-isobutoxybenzyl)-9-[4-(1,3-oxazol-5-yl)benzoyl]- | C | 7.38 | 488 |
| 3,9-diazaspiro[5.5]undecane | | | |
| 3-(2-isobutoxybenzyl)-9-[4-(1H-1,2,4-triazol-1- | C | 6.9 | 488 |
| yl)benzoyl]-3,9-diazaspiro[5.5]undecane | | | |
| 3-(4-chlorobenzoyl)-9-(3-isobutoxybenzyl)-3,9- | C | 8 | 455/457 |
| diazaspiro[5.5]undecane | | | |
| 3-(2-isobutoxybenzyl)-9-[(5-methyl-2-thienyl)carbonyl]- | C | 7.72 | 441 |
| 3,9-diazaspiro[5.5]undecane | | | |
| 3-(4-chlorobenzoyl)-9-[(3-phenoxy-2-thienyl)methyl]- | C | 7.73 | 481/483 |
| 3,9-diazaspiro[5.5]undecane | | | |
| 3-(2-isobutoxybenzyl)-9-[4-(trifluoromethyl)benzoyl]- | C | 8.24 | 489 |
| 3,9-diazaspiro[5.5]undecane | | | |
| 3-[(6-chloropyridin-2-yl)carbonyl]-9-(2- | C | 7.26 | 456/458 |
| isobutoxybenzyl)-3,9-diazaspiro[5.5]undecane | | | |
| 3-(2-isobutoxybenzyl)-9-[(6-methylpyridin-3- | C | 5.88 | 436 |
| yl)carbonyl]-3,9-diazaspiro[5.5]undecane | | | |
| 3-[(6-chloropyridin-3-yl)carbonyl]-9-(2- | C | 7.2 | 456/458 |
| isobutoxybenzyl)-3,9-diazaspiro[5.5]undecane | | | |
| 3-(2-chloroisonicotinoyl)-9-(2-isobutoxybenzyl)-3,9- | C | 7.16 | 456/458 |
| diazaspiro[5.5]undecane | | | |
| 3-(2-isobutoxybenzyl)-9-(quinolin-2-ylcarbonyl)-3,9- | C | 7.63 | 472 |
| diazaspiro[5.5]undecane | | | |
| 2-[3-(4-chlorophenyl)propanoyl]-7-(2-phenoxybenzyl)- | C | 7.9 | 475/477 |
| 2,7-diazaspiro[3.5]nonane | | | |
| 3-(2-isobutoxybenzyl)-9-[(1-oxidopyridin-3-yl)carbonyl]- | C | 5.98 | 438 |
| 3,9-diazaspiro[5.5]undecane | | | |
| 3-[3-(pyridin-4-ylmethoxy)benzyl]-9-(pyrimidin-4- | C | 3.79 | 458 |
| ylcarbonyl)-3,9-diazaspiro[5.5]undecane | | | |
| 2-(4-chlorobenzoyl)-9-(2-isobutoxybenzyl)-2,9- | F | 2.45 | 455/457 |
| diazaspiro[5.5]undecane | | | |
| 9-(2-isobutoxybenzyl)-2-isonicotinoyl-2,9- | F | 1.91 | 422 |
| diazaspiro[5.5]undecane | | | |
| 2-(3-furoyl)-9-(2-isobutoxybenzyl)-2,9- | F | 2.21 | 411 |
| diazaspiro[5.5]undecane | | | |
| 9-(2-isobutoxybenzyl)-2-(quinolin-2-ylcarbonyl)-2,9- | F | 2.42 | 472 |
| diazaspiro[5.5]undecane | | | |
| 9-(2-isobutoxybenzyl)-2-(pyridin-4-ylacetyl)-2,9- | F | 1.82 | 436 |
| diazaspiro[5.5]undecane | | | |
| 7-(4-chlorobenzoyl)-2-(2-isobutoxybenzyl)-2,7- | F | 2.48 | 441/443 |
| diazaspiro[4.5]decane | | | |
| 2-(2-isobutoxybenzyl)-7-isonicotinoyl-2,7- | F | 1.94 | 408 |
| diazaspiro[4.5]decane | | | |
| 7-(3-furoyl)-2-(2-isobutoxybenzyl)-2,7- | F | 2.15 | 397 |
| diazaspiro[4.5]decane | | | |
| 2-{[2-(2-isobutoxybenzyl)-2,7-diazaspiro[4.5]dec-7- | F | 2.34 | 458 |
| yl]carbonyl}quinoline | | | |
| 2-(2-isobutoxybenzyl)-7-(pyridin-4-ylacetyl)-2,7- | F | 1.76 | 422 |
| diazaspiro[4.5]decane | | | |
| 2-(4-chlorobenzoyl)-7-(2-isobutoxybenzyl)-2,7- | E | 2.53 | 427/429 |
| diazaspiro[4.4]nonane | | | |
| 2-(2-isobutoxybenzyl)-7-isonicotinoyl-2,7- | F | 1.83 | 394 |
| diazaspiro[4.4]nonane | | | |
| 2-(3-furoyl)-7-(2-isobutoxybenzyl)-2,7- | E | 2.28 | 383 |
| diazaspiro[4.4]nonane | | | |
| 2-{[7-(2-isobutoxybenzyl)-2,7-diazaspiro[4.4]non-2- | F | 2.23 | 444 |
| yl]carbonyl}quinoline | | | |
| 2-(2-isobutoxybenzyl)-7-(pyridin-4-ylacetyl)-2,7- | F | 1.72 | 408 |
| diazaspiro[4.4]nonane | | | |
| 2-[(4-chlorophenyl)acetyl]-7-(2-isobutoxybenzyl)-2,7- | F | 2.4 | 441/443 |
| diazaspiro[3.5]nonane | | | |
| 2-[3-(4-chlorophenyl)propanoyl]-7-(3-phenoxybenzyl)- | F | 2.35 | 475/477 |
| 2,7-diazaspiro[3.5]nonane | | | |
| 2-[3-(4-chlorophenyl)propanoyl]-7-(2-isobutoxybenzyl)- | E | 2.6 | 455/457 |
| 2,7-diazaspiro[3.5]nonane | | | |
| 2-[(4-chlorophenyl)acetyl]-7-(2-isobutoxybenzyl)-2,7- | C | 7.74 | 441/443 |
| diazaspiro[3.5]nonane | | | |
| 2-(4-chlorophenyl)-7-(3-isobutoxybenzyl)-2,7- | E | 2.53 | 427/429 |
| diazaspiro[4.4]nonane | | | |
| 2-(4-chlorobenzoyl)-7-(2-phenoxybenzyl)-2,7- | E | 2.46 | 447/449 |
| diazaspiro[4.4]nonane | | | |
| 2-[2-(benzyloxy)benzyl]-7-(4-chlorobenzoyl)-2,7- | E | 2.51 | 461/463 |
| diazaspiro[4.4]nonane | | | |
| 3-(2-isobutoxybenzyl)-9-(quinolin-3-ylcarbonyl)-3,9- | C | 7.11 | 472 |
| diazaspiro[5.5]undecane | | | |
| 3-(2-isobutoxybenzyl)-9-(pyridin-4-ylacetyl)-3,9- | C | 5.51 | 436 |
| diazaspiro[5.5]undecane | | | |
| 8-(2-isobutoxybenzyl)-2-(pyridin-3-ylacetyl)-2,8- | C | 5.26 | 422 |
| diazaspiro[4.5]decane | | | |
| 8-(2-isobutoxybenzyl)-2-(pyridin-4-ylacetyl)-2,8- | C | 5.4 | 422 |
| diazaspiro[4.5]decane | | | |
| 7-(2-isobutoxybenzyl)-2-(pyridin-2-ylacetyl)-2,7- | C | 5.64 | 408 |
| diazaspiro[3.5]nonane | | | |
| 7-(2-isobutoxybenzyl)-2-(pyridin-3-ylacetyl)-2,7- | C | 5.35 | 408 |
| diazaspiro[3.5]nonane | | | |
| 8-(2-isobutoxybenzyl)-2-(pyridin-3-ylcarbonyl)-2,8- | C | 6.33 | 408 |
| diazaspiro[4.5]decane | | | |
| 8-(2-isobutoxybenzyl)-2-isonicotinoyl-2,8- | C | 6.04 | 408 |
| diazaspiro[4.5]decane | | | |
| 7-(2-isobutoxybenzyl)-2-(pyridin-4-ylacetyl)-2,7- | C | 5.33 | 408 |
| diazaspiro[3.5]nonane | | | |
| 8-(2-isobutoxybenzyl)-2-(pyridin-2-ylcarbonyl)-2,8- | C | 6.72 | 408 |
| diazaspiro[4.5]decane | | | |
| 3-(2-isobutoxybenzyl)-9-(pyridin-2-ylacetyl)-3,9- | B | 3.71 | 436 |
| diazaspiro[5.5]undecane | | | |
| 3-(2-isobutoxybenzyl)-9-(pyridin-3-ylacetyl)-3,9- | B | 3.47 | 436 |
| diazaspiro[5.5]undecane | | | |
| 3-(2-isobutoxybenzyl)-9-[4-(2H-tetrazol-5-yl)benzoyl]- | B | 4.74 | 489 |
| 3,9-diazaspiro[5.5]undecane | | | |
| 7-(2-isobutoxybenzyl)-2-(pyridin-2-ylcarbonyl)-2,7- | C | 7.12 | 394 |
| diazaspiro[3.5]nonane | | | |
| 7-(2-isobutoxybenzyl)-2-(pyridin-3-ylcarbonyl)-2,7- | C | 6.47 | 394 |
| diazaspiro[3.5]nonane | | | |
| 7-(2-isobutoxybenzyl)-2-isonicotinoyl-2,7- | C | 6.26 | 394 |
| diazaspiro[3.5]nonane | | | |
| 3-(2-isobutoxybenzyl)-9-(1-oxidoisonicotinoyl)-3,9- | C | 6.9 | 438 |
| diazaspiro[5.5]undecane | | | |
| 3-(2-isobutoxybenzyl)-9-(quinoxalin-2-ylcarbonyl)-3,9- | C | 7.7 | 473 |
| diazaspiro[5.5]undecane | | | |
| 3-[4-(1H-imidazol-1-yl)benzoyl]-9-(2-isobutoxybenzyl)- | C | 7.55 | 487 |
| 3,9-diazaspiro[5.5]undecane | | | |
| 5-{[9-(2-isobutoxybenzyl)-3,9-diazaspiro[5.5]undec-3- | C | 6.44 | 438 |
| yl]carbonyl}pyridin-2(1H)-one | | | |
| 3-{[9-(2-isobutoxybenzyl)-3,9-diazaspiro[5.5]undec-3- | C | 7.93 | 438 |
| yl]carbonyl}pyridin-2(1H)-one | | | |
| 3-(2-isobutoxybenzyl)-9-[3-(2H-tetrazol-5-yl)benzoyl]- | C | 7.31 | 489 |
| 3,9-diazaspiro[5.5]undecane | | | |
| 3-(2-isobutoxybenzyl)-9-(2-methylisonicotinoyl)-3,9- | C | 5.79 | 436 |
| diazaspiro[5.5]undecane | | | |
| 3-[2-(cyclopropylmethoxy)benzyl]-9-isonicotinoyl-3,9- | C | 6.64 | 420 |
| diazaspiro[5.5]undecane | | | |
| 3-[1-(2-isobutoxyphenyl)ethyl]-9-isonicotinoyl-3,9- | C | 7.2 | 436 |
| diazaspiro[5.5]undecane | | | |
| 3-[(6-isobutoxypyridin-2-yl)methyl]-9-isonicotinoyl-3,9- | C | 6.49 | 423 |
| diazaspiro[5.5]undecane | | | |
| 3-[(6-isobutoxypyridin-2-yl)methyl]-9-(pyrimidin-4- | C | 6.98 | 424 |
| ylcarbonyl)-3,9-diazaspiro[5.5]undecane | | | |
| 3-isonicotinoyl-9-{2-[(2-methylprop-2-en-1- | C | 6.58 | 420 |
| yl)oxy]benzyl}-3,9-diazaspiro[5.5]undecane | | | |
| 3-isonicotinoyl-9-(2-phenoxybenzyl)-3,9- | C | 6.93 | 442 |
| diazaspiro[5.5]undecane | | | |
| 3-(2-isobutoxybenzyl)-9-[2-(2H-tetrazol-5-yl)benzoyl]- | C | 8.12 | 489 |
| 3,9-diazaspiro[5.5]undecane | | | |
| 3-isonicotinoyl-9-[2-(1,1,2,2-tetrafluoroethoxy)benzyl]- | C | 6.04 | 466 |
| 3,9-diazaspiro[5.5]undecane | | | |
| 4-{[9-(2-isobutoxybenzyl)-3,9-diazaspiro[5.5]undecane- | C | 6.65 | 500 |
| 3-yl]carbonyl}benzene sulfonamide | | | |

### EXAMPLE: 8

### 8-(2-isobutoxybenzyl)-2-(pyridin-2-ylacetyl)-2,8-diazaspiro[4.5]decane

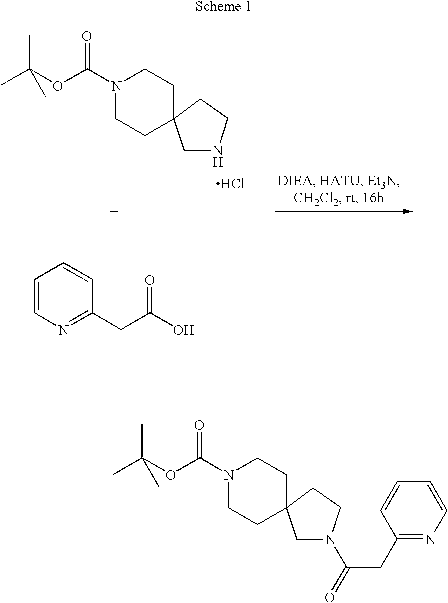

### a) tert-butyl 2-(pyridin-2-ylacetyl)-2,8-diazaspiro[4.5]decane-8-carboxylate

To a solution of tert-butyl 2,8-diazaspiro[4.5]decane-8-carboxylate hydrochloride (800 mg, 2.89 mmol, 1 equiv), 2-pyridylacetic acid hydrochloride (500 mg, 2.89 mmol, 1 equiv) and triethylamine (1.2 ml, 8.68 mmol, 3 equiv) in dry dichloromethane (12 ml), was added O-(7-azabenzotriazol-1-yl)-N,N,N′,N′-tetramethyluronium hexafluorophosphate (1.1 g, 2.89 mmol, 1 equiv). The reaction mixture was stirred at room temperature for 3 hours, then concentrated, and partitioned between ethyl acetate and saturated sodium hydrogen carbonate. The organic layer was isolated, dried (MgSO₄) and concentrated to give a dark orange oil which was subjected to silica-gel chromatography using a mixture of methanol and dichloromethane (4:96, v/v) as eluent, to provide the title compound (1.2 g, quantitative) as a dark yellow oil. LCMS Method E): R_{T}=2.19 minutes; 360 (M+H)⁺.

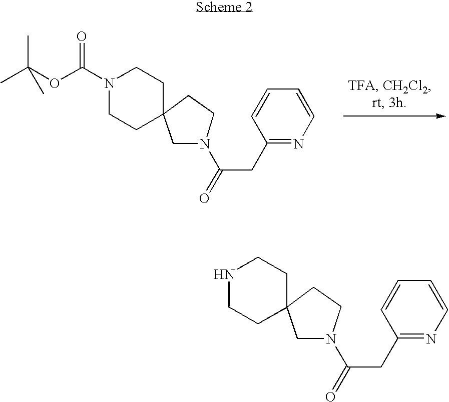

### b) 2-(pyridin-2-ylacetyl)-2,8-diazaspiro[4.5]decane

To a solution of tert-butyl 2-(pyridin-2-ylacetyl)-2,8-diazaspiro[4.5]decane-8-carboxylate (1.04 g, 2.89 mmol) in dichloromethane (4 ml) was added trifluoroacetic acid (2 ml). After stirring for 3 hours the reaction mixture was concentrated to an oil, which was dissolved in ethyl acetate and washed with 1M sodium hydroxide solution. The organic layer was isolated and the aqueous layer washed with dichloromethane (2×), followed by ethyl acetate (2×). The combined organic layers were concentrated to provide the title compound (250 mg, 33%) as a yellow oil. LCMS (Method E): R_{T}=0.34 minutes; 260 (M+H)⁺.

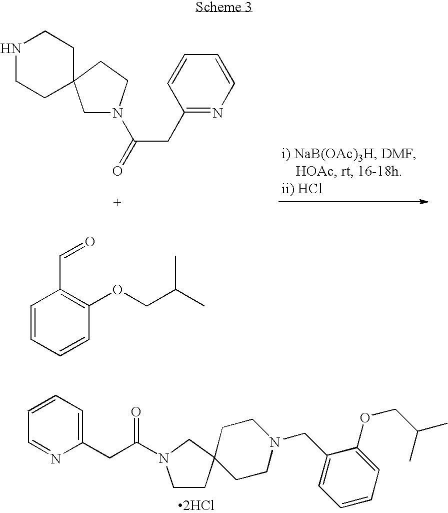

### c) 8-(2-isobutoxybenzyl)-2-(pyridin-2-ylacetyl)-2,8-diazaspiro[4.5]decane

A solution of 2-(pyridin-2-ylacetyl)-2,8-diazaspiro[4.5]decane (250 mg, 0.90 mmol) and 2-isobutoxybenzaldehyde (160 mg, 0.90 mmol) in dichloroethane (5 ml), was stirred at room temperature for 1.5 hours before sodium triacetoxyborohydride (286 mg, 1.35 mmol) was added. After stirring overnight the reaction mixture was concentrated to give an orange oil, which was subjected to silica-gel column chromatography using methanol and dichloromethane (4:96, v/v) as eluent to provide a yellow oil. The yellow oil was triturated with 1M hydrochloric acid in methanol to obtain the title compound (80 mg, 18%) as a pale yellow solid. LCMS (Method B): R_{T}=3.66 minutes; 422 (M+H)⁺.

### EXAMPLE: 9

### 3-(4-chlorobenzoyl)-9-[(2-isobutoxypyridin-3-yl)methyl]-3,9-diazaspiro[5.5]undecane dihydrochloride

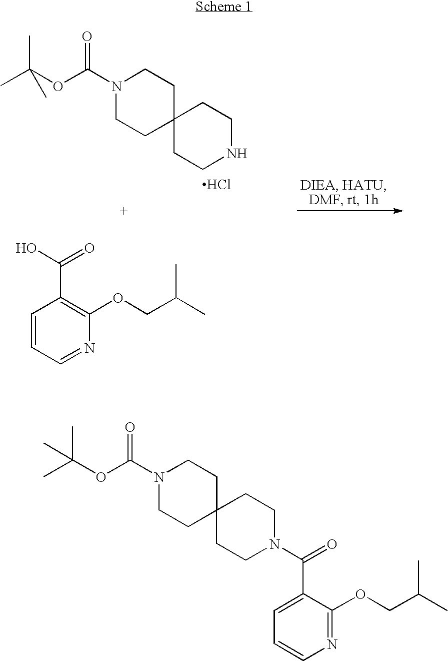

### a) tert-butyl 9-[(2-isobutoxypyridin-3-yl)carbonyl]-3,9-diazaspiro[5.5]undecane-3-carbonylate

To a solution tert-butyl 3,9-diazaspiro[5.5]undecane-3-carboxylate hydrochloride (293 mg, 1.0 mmol), 2-isobutoxynicotinic acid (214 mg, 1.1 mmol) and diisopropylethylamine (0.385 μl, 2.2 mmol) in dry N,N-dimethylformamide (9.5 ml) was added O-(7-azabenzotriazol-1-yl)-N,N,N′,N′-tetramethyluronium hexafluorophosphate (400 mg, 1.05 mmol). After stirring the solution for 1 hour, the reaction mixture was poured onto saturated sodium hydrogen carbonate, and extracted with ethyl acetate (2×). The combined organic layers were washed with water, brine, then dried (Na₂SO₄), and concentrated to a viscous gum. The gum was subjected to silica-gel column chromatography using a mixture of ethyl acetate and cyclohexane (gradient 25:75 to 70:30, v/v) as eluent to provide the title compound (403 mg, 94%) as a colourless viscous gum.

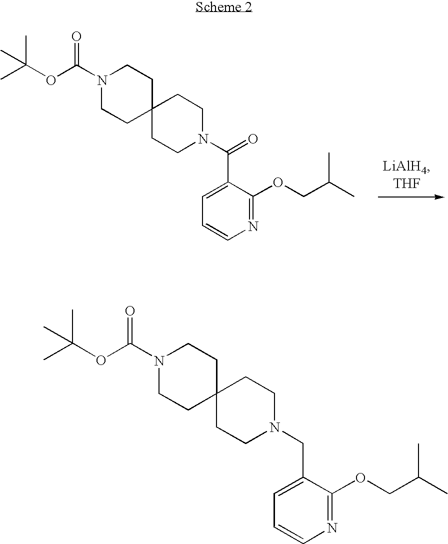

### b) tert-butyl 9-[(2-isobutoxypyridin-3-yl)methyl]-3,9-diazaspiro[5.5]undecane-3-carboxylate

To a solution of tert-butyl 9-[(2-isobutoxypyridin-3-yl)carbonyl]-3,9-diazaspiro[5.5]undecane-3-carboxylate (100 mg, 0.23 mmol) in dry tetrahydrofuran (2.5 ml) under nitrogen was added lithium aluminium hydride (18 mg, 0.47 mmol). After stirring at room temperature for 1 hour the reaction mixture was quenched with saturated ammonium chloride solution, and the resultant mixture was extracted with ethyl acetate (3×). The combined organic layers were washed with brine and concentrated to give a gum, which was subjected to chromatography using an Isolute® flash SCX-2 cartridge using 2M ammonia in methanol as eluent, to provide the title compound (46 mg, 48%) as a colourless oil. LCMS (Method E): R_{T}=2.45 minutes; 418 (M+H)⁺.

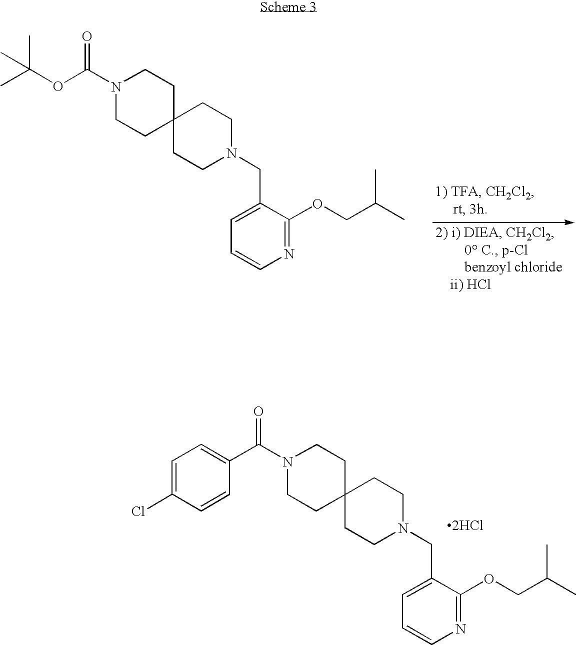

### c) 3-(4-chlorobenzoyl)-9-[(2-isobutoxypyridin-3-yl)methyl]-3,9-diazaspiro[5.5]undecane dihydrochloride

To a solution of tert-butyl 9-[(2-isobutoxypyridin-3-yl)methyl]-3,9-diazaspiro[5.5]undecane-3-carboxylate (43 mg, 0.1 mmol) in dry dichloromethane (3 ml) was added trifluoroacetic acid (1 ml). After stirring for 3 hours the reaction mixture was concentrated to give an oily residue. The oily residue was suspended in dichloromethane (3 ml) at 0° C., to which diisopropylethylamine (0.179 μl, 1.0 mmol) was added, followed by 4-chlorobenzoyl chloride (22 mg, 0.126 mmol). The reaction mixture was allowed to stir overnight before being concentrated and subjected to silica-gel column chromatography using a mixture of ethyl acetate and triethylamine (97:3, v/v) to give a light brown oil. The oil was triturated with 2M hydrochloric acid in diethyl ether, to provide the title compound (50 mg, 95%) as a white solid.

¹H NMR (400 MHz, CD₃OD with NaOD added): δ 8.02 (dd, 1H), 7.69 (dd, 1H), 7.47 (m, 2H), 7.39 (m, 2H), 6.95 (dd, 1H), 4.06 (d, 2H), 3.71 (br m, 2H), 3.57 (s, 2H), 3.38 (br m, 2H), 2.52 (br m, 4H), 2.08 (m, 1H), 1.60 (br m, 6H), 1.45 (br m, 2H), 1.04 (d, 61. LCMS (Method C): R_{T}=7.33 minutes; 456 & 458 (M+H)⁺.

The following compounds were prepared according to the general procedure used for example 9.

| | LCMS | Retention | Mass Ion/ |
|---|---|---|---|
| Compound | Method | time/min | MH⁺ |
| 3-[(2-isobutoxypyridin-3-yl)methyl]- | C | 6.13 | 423 |
| 9-isonicotinoyl-3,9- | | | |
| diazaspiro[5.5]undecane | | | |
| 3-[(2-isobutoxypyridin-3-yl)methyl]-9- | C | 6.14 | 424 |
| (pyrimidin-4-ylcarbonyl)-3,9- | | | |
| diazaspiro[5.5]undecane | | | |

### EXAMPLE: 10

### 9-(2-isobutoxybenzyl)-N-3-thienyl-3,9-diazaspiro[5.5]undecane-3-carboxamide

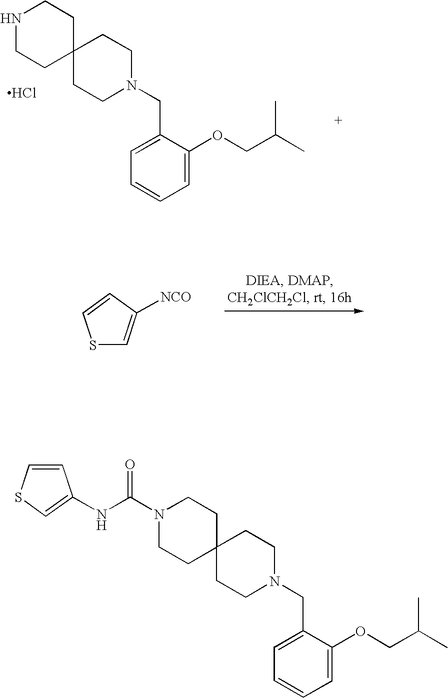

A solution of 3-(2-isobutoxybenzyl)-3,9-diazaspiro[5.5]undecane dihydrochloride (32 mg, 0.10 mmol) and 3-thienyl isocyanate (38 mg, 0.30 mmol) in dichloromethane (1 ml) was stirred for 18 hours. Polymer-bound tris(2-aminoethyl)amine (100 mg) was added to the reaction mixture, which was stirred for a further 1 hour before being filtered. The filtrate was concentrated and subjected to purification with an Isolute® flash SCX-2 cartridge using methanol and dichloromethane (1:1, v/v) followed by 0.5M ammonia in methanol as eluent, to provide the title compound (40 mg, 89%).

¹H NMR (400 MHz, CD₃OD): δ 7.29 (dd, 1H), 7.23 (m, 2H), 7.15 (dd, 1H), 7.05 (dd, 1H), 6.93 (d, 1H), 6.90 (td, 1H), 3.76 (d, 2H), 3.64 (s, 2H), 3.46 (br m, 4H), 2.55 (br m, 4H), 2.10 (m, 1H), 1.58 (br m, 4H), 1.49 (br m, 4H), 1.07 (d, 6H). LCMS (Method F): R_{T}=2.28 minutes; 442 (M+H)⁺.

The following compounds were prepared according to the general procedure used for example 10.

| | LCMS | Retention | Mass Ion/ |
|---|---|---|---|
| Compound | Method | time/min | MH⁺ |
| N-(4-chlorophenyl)-9-(2-isobutoxybenzyl)-3,9- | D | 2.89 | 470 |
| diazaspiro[5.5]undecane-3-carboxamide | | | |
| 9-(2-isobutoxybenzyl)-N-(2-phenylethyl)-3,9- | D | 2.75 | 464 |
| diazaspiro[5.5]undecane-3-carboxamide | | | |
| 9-(2-isobutoxybenzyl)-N-[2-(2-thienyl)ethyl]-3,9- | D | 2.73 | 470 |
| diazaspiro[5.5]undecane-3-carboxamide | | | |
| 9-(2-isobutoxybenzyl)-N-2-thienyl-3,9- | D | 2.7 | 442 |
| diazaspiro[5.5]undecane-3-carboxamide | | | |
| N-(2,3-dihydro-1-benzofuran-6-yl)-9-(2-isobutoxybenzyl)-3,9- | D | 2.69 | 478 |
| diazaspiro[5.5]undecane-3-carboxamide | | | |
| N-(2,3-dihydro-1,4-benzodioxin-6-yl)-9-(2-isobutoxybenzyl)- | D | 2.72 | 494 |
| 3,9-diazaspiro[5.5]undecane-3-carboxamide | | | |
| 9-(2-isobutoxybenzyl)-N-(5-methyl-3-phenylisoxazol-4-yl)-3,9- | D | 2.72 | 517 |
| diazaspiro[5.5]undecane-3-carboxamide | | | |
| 9-(2-isobutoxybenzyl)-N-(3-methyl-5-phenylisoxazol-4-yl)-3,9- | D | 2.75 | 517 |
| diazaspiro[5.5]undecane-3-carboxamide | | | |
| N-(2,6-dichloropyridin-4-yl)-9-(2-isobutoxybenzyl)-3,9- | D | 2.87 | 505/507 |
| diazaspiro[5.5]undecane-3-carboxamide | | | |
| N-2,1,3-benzothiadiazol-4-yl-9-(2-isobutoxybenzyl)-3,9- | C | 7.91 | 494 |
| diazaspiro[5.5]undecane-3-carboxamide | | | |
| 9-(2-isobutoxybenzyl)-N-(4-phenoxyphenyl)-3,9- | D | 3.04 | 528 |
| diazaspiro[5.5]undecane-3-carboxamide | | | |
| 9-(2-isobutoxybenzyl)-N-(2-phenylcyclopropyl)-3,9- | D | 2.84 | 476 |
| diazaspiro[5.5]undecane-3-carboxamide | | | |
| 9-(2-isobutoxybenzyl)-N-(tetrahydro-2H-pyran-2-yl)-3,9- | C | 6.76 | 444 |
| diazaspiro[5.5]undecane-3-carboxamide | | | |
| 9-(2-isobutoxybenzyl)-N-(phenyl)-3,9- | F | 2.34 | 436 |
| diazaspiro[5.5]undecane-3-carboxamide | | | |
| N-benzyl-9-(2-isobutoxybenzyl)-3,9-diazaspiro[5.5]undecane- | F | 2.36 | 450 |
| 3-carboxamide | | | |
| N-cyclohexyl-9-(2-isobutoxybenzyl)-3,9- | F | 2.35 | 442 |
| diazaspiro[5.5]undecane-3-carboxamide | | | |
| N-(tert-butyl)-9-(2-isobutoxybenzyl)-3,9- | F | 2.22 | 416 |
| diazaspiro[5.5]undecane-3-carboxamide | | | |
| ethyl N-{[9-(2-isobutoxybenzyl)-3,9-diazaspiro[5.5]undec-3- | F | 2.11 | 446 |
| yl]carbonyl}glycinate | | | |
| N-cyclopentyl-9-(2-isobutoxybenzyl)-3,9- | D | 2.8 | 428 |
| diazaspiro[5.5]undecane-3-carboxamide | | | |
| N-(2,4-dichlorobenzyl)-9-(2-isobutoxybenzyl)-3,9- | D | 3.04 | 518/520 |
| diazaspiro[5.5]undecane-3-carboxamide | | | |
| 9-(2-isobutoxybenzyl)-N-(2-methoxyphenyl)-3,9- | F | 2.39 | 466 |
| diazaspiro[5.5]undecane-3-carboxamide | | | |
| 9-(2-isobutoxybenzyl)-N-(4-methoxyphenyl)-3,9- | D | 2.84 | 466 |
| diazaspiro[5.5]undecane-3-carboxamide | | | |
| ethyl 4-({[9-(2-isobutoxybenzyl)-3,9-diazaspiro[5.5]undec-3- | D | 3.01 | 508 |
| yl]carbonyl}amino)benzoate | | | |
| ethyl 3-({[9-(2-isobutoxybenzyl)-3,9-diazaspiro[5.5]undec-3- | F | 2.53 | 508 |
| yl]carbonyl}amino)benzoate | | | |
| N-(3-chlorophenyl)-9-(2-isobutoxybenzyl)-3,9- | D | 3.05 | 470/472 |
| diazaspiro[5.5]undecane-3-carboxamide | | | |
| 9-(2-isobutoxybenzyl)-N-(4-methoxybenzyl)-3,9- | D | 2.86 | 480 |
| diazaspiro[5.5]undecane-3-carboxamide | | | |
| N-[2-(4-ethylphenyl)ethyl]-9-(2-isobutoxybenzyl)-3,9- | D | 3.11 | 492 |
| diazaspiro[5.5]undecane-3-carboxamide | | | |
| 9-(2-isobutoxybenzyl)-N-(4-isopropylphenyl)-3,9- | D | 3.13 | 478 |
| diazaspiro[5.5]undecane-3-carboxamide | | | |
| N-(3-cyanophenyl)-9-(2-isobutoxybenzyl)-3,9- | D | 2.91 | 461 |
| diazaspiro[5.5]undecane-3-carboxamide | | | |
| 9-(2-isobutoxybenzyl)-N-(2-methylphenyl)-3,9- | F | 2.33 | 450 |
| diazaspiro[5.5]undecane-3-carboxamide | | | |
| 9-(2-isobutoxybenzyl)-N-(3-methylphenyl)-3,9- | D | 2.94 | 450 |
| diazaspiro[5.5]undecane-3-carboxamide | | | |
| 9-(2-isobutoxybenzyl)-N-(4-methylphenyl)-3,9- | D | 2.95 | 450 |
| diazaspiro[5.5]undecane-3-carboxamide | | | |
| N-(2,6-dichlorophenyl)-9-(2-isobutoxybenzyl)-3,9- | F | 2.38 | 504/506 |
| diazaspiro[5.5]undecane-3-carboxamide | | | |
| N-(3,4-dichlorophenyl)-9-(2-isobutoxybenzyl)-3,9- | F | 2.65 | 504/506 |
| diazaspiro[5.5]undecane-3-carboxamide | | | |
| N-(3,5-dichlorophenyl)-9-(2-isobutoxybenzyl)-3,9- | F | 2.71 | 504/506 |
| diazaspiro[5.5]undecane-3-carboxamide | | | |
| N-(4-chlorophenyl)-9-(2-isobutoxybenzyl)-2,9- | E | 2.69 | 470/472 |
| diazaspiro[5.5]undecane-2-carboxamide | | | |
| N-(4-chlorophenyl)-2-(2-isobutoxybenzyl)-2,7- | E | 2.68 | 456/458 |
| diazaspiro[4.5]decane-7-carboxamide | | | |
| N-(4-chlorophenyl)-7-(2-isobutoxybenzyl)-2,7- | E | 2.54 | 442/444 |
| diazaspiro[4.4]nonane-2-carboxamide | | | |
| N-(4-chlorophenyl)-7-(2-isobutoxybenzyl)-2,7- | E | 2.57 | 442/444 |
| diazaspiro[3.5]nonane-2-carboxamide | | | |
| 9-(2-isobutoxybenzyl)-N-[(2-methylphenyl)sulfonyl]-3,9- | F | 2.34 | 514 |
| diazaspiro[5.5]undecane-3-carboxamide | | | |
| N-[(4-chlorophenyl)sulfonyl]-9-(2-isobutoxybenzyl)-3,9- | F | 2.44 | 534/536 |
| diazaspiro[5.5]undecane-3-carboxamide | | | |
| 9-(2-isobutoxybenzyl)-N-[(2-methylphenyl)sulfonyl]-3,9- | F | 2.34 | 514 |
| diazaspiro[5.5]undecane-3-carboxamide | | | |
| N-[(4-chlorophenyl)sulfonyl]-9-(2-isobutoxybenzyl)-2,9- | F | 2.48 | 534/536 |
| diazaspiro[5.5]undecane-2-carboxamide | | | |

### EXAMPLE: 11

### 3-(2-isobutoxybenzyl)-9-(2-thienylsulfonyl)-3,9-diazaspiro[5.5]undecane

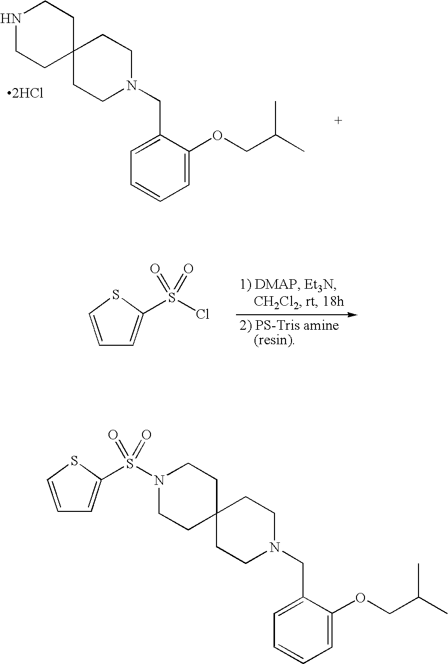

A solution of 3-(2-isobutoxybenzyl)-3,9-diazaspiro[5.5]undecane dihydrochloride (32 mg, 0.10 mmol), thiophene-2-sulfonyl chloride (55 mg, 0.30 mmol), triethylamine (40 μl, 0.30 mmol), 4-dimethylaminopyridine (2.4 mg, 0.02 mmol) in dichloromethane (2 ml) was stirred for 18 hours. Polymer-bound tris(2-aminoethyl)amine (160 mg) was added to the reaction mixture, which was stirred for a further 3 hours before being filtered. The filtrate was concentrated and subjected to purification with an Isolute® flash SCX-2 cartridge using methanol and dichloromethane (1:1, v/v) followed by 0.5M ammonia in methanol as eluent, to provide the title compound (19.3 mg, 42%).

¹H NMR (400 MHz, CD₃OD): δ 7.82 (dd, 1H), 7.57 (dd, 1H), 7.23 (m, 3H), 6.90 (d, 1H), 6.87 (td, 1H), 3.73 (d, 2H), 3.58 (s, 2H), 3.01 (br t, 4H), 2.46 (br t, 4H), 2.07 (m, 1H), 1.56 (br t, 4H), 1.41 (br t, 4H), 1.04 (d, 6H). LCMS (Method E): R_{T}=2.55 minutes; 463 (M+H)⁺.

The following compounds were prepared according to the general procedure used for example 11.

| | | Retention | Mass Ion/ |
|---|---|---|---|
| Compound | LCMS Method | time/min | MH⁺ |
| 3-(2-isobutoxybenzyl)-9-(phenylsulfonyl)-3,9- | F | 2.39 | 457 |
| diazaspiro[5.5]undecane | | | |
| 3-(2-isobutoxybenzyl)-9-(propylsulfonyl)-3,9- | E | 2.4 | 423 |
| diazaspiro[5.5]undecane | | | |
| 3-(2-isobutoxybenzyl)-9-[(3-methylphenyl)sulfonyl]-3,9- | E | 2.63 | 471 |
| diazaspiro[5.5]undecane | | | |
| 3-(benzylsulfonyl)-9-(2-isobutoxybenzyl)-3,9- | E | 2.59 | 471 |
| diazaspiro[5.5]undecane | | | |
| 3-(2-isobutoxybenzyl)-9-(isopropylsulfonyl)-3,9- | D | 2.63 | 423 |
| diazaspiro[5.5]undecane | | | |
| 3-(2-isobutoxybenzyl)-9-(3-thienylsulfonyl)-3,9- | D | 2.77 | 463 |
| diazaspiro[5.5]undecane | | | |
| 3-[(2,5-dimethyl-3-furyl)sulfonyl]-9-(2-isobutoxybenzyl)- | D | 2.9 | 475 |
| 3,9-diazaspiro[5.5]undecane | | | |
| 3-[(3,5-dimethylisoxazol-4-yl)sulfonyl]-9-(2- | D | 2.8 | 476 |
| isobutoxybenzyl)-3,9-diazaspiro[5.5]undecane | | | |
| 2-{[9-(2-isobutoxybenzyl)-3,9-diazaspiro[5.5]undec-3- | D | 2.8 | 482 |
| yl]sulfonyl}benzonitrile | | | |
| 4-{[9-(2-isobutoxybenzyl)-3,9-diazaspiro[5.5]undec-3- | D | 2.76 | 482 |
| yl]sulfonyl}benzonitrile | | | |
| 3-[(2,5-dimethoxyphenyl)sulfonyl]-9-(2- | D | 2.76 | 517 |
| isobutoxybenzyl)-3,9-diazaspiro[5.5]undecane | | | |
| 3-(2-isobutoxybenzyl)-9-[(4-methoxyphenyl)sulfonyl]- | D | 2.85 | 487 |
| 3,9-diazaspiro[5.5]undecane | | | |
| 3-(2-isobutoxybenzyl)-9-[(3-nitrophenyl)sulfonyl]-3,9- | D | 2.94 | 502 |
| diazaspiro[5.5]undecane | | | |
| 3-[(2-chlorophenyl)sulfonyl]-9-(2-isobutoxybenzyl)-3,9- | D | 2.92 | 491/493 |
| diazaspiro[5.5]undecane | | | |
| 3-[(4-chlorophenyl)sulfonyl]-9-(2-isobutoxybenzyl)-3,9- | D | 2.98 | 491/493 |
| diazaspiro[5.5]undecane | | | |
| 3-[(2,4-dimethyl-1,3-thiazol-5-yl)sulfonyl]-9-(2- | D | 2.7 | 492 |
| isobutoxybenzyl)-3,9-diazaspiro[5.5]undecane | | | |
| 3-(2,1,3-benzoxadiazol-4-ylsulfonyl)-9-(2- | D | 2.79 | 499 |
| isobutoxybenzyl)-3,9-diazaspiro[5.5]undecane | | | |
| 2-[(4-chlorophenyl)sulfonyl]-9-(2-isobutoxybenzyl)-2,9- | F | 2.66 | 491/493 |
| diazaspiro[5.5]undecane | | | |
| 7-[(4-chlorophenyl)sulfonyl]-2-(2-isobutoxybenzyl)-2,7- | F | 2.61 | 477/479 |
| diazaspiro[4.5]decane | | | |
| 2-[(4-chlorophenyl)sulfonyl]-7-(2-isobutoxybenzyl)-2,7- | F | 2.53 | 463/465 |
| diazaspiro[4.4]nonane | | | |
| 2-[(4-chlorophenyl)sulfonyl]-7-(2-isobutoxybenzyl)-2,7- | F | 2.5 | 463/465 |
| diazaspiro[3.5]nonane | | | |
| 3-(2-isobutoxybenzyl)-9-[(4-isopropylphenyl)sulfonyl]- | D | 3.09 | 499 |
| 3,9-diazaspiro[5.5]undecane | | | |
| 4-{[9-(2-isobutoxybenzyl)-3,9-diazaspiro[5.5]undec-3- | D | 2.7 | 501 |
| yl]sulfonyl}benzoic acid | | | |
| 3-(2-isobutoxybenzyl)-9-(quinolin-8-ylsulfonyl)-3,9- | D | 2.82 | 508 |
| diazaspiro[5.5]undecane | | | |
| 3-[(5-chloro-1,3-dimethyl-1H-pyrazol-4-yl)sulfonyl]-9- | D | 2.73 | 509/511 |
| (2-isobutoxybenzyl)-3,9-diazaspiro[5.5]undecane | | | |
| 3-[(4-tert-butylphenyl)sulfonyl]-9-(2-isobutoxybenzyl)- | D | 3.16 | 513 |
| 3,9-diazaspiro[5.5]undecane | | | |
| N-(4-{[9-(2-isobutoxybenzyl)-3,9-diazaspiro[5.5]undec- | D | 2.68 | 514 |
| 3-yl]sulfonyl}phenyl)acetamide | | | |
| 3-(2,1,3-benzothiadiazol-4-ylsulfonyl)-9-(2- | D | 2.82 | 515 |
| isobutoxybenzyl)-3,9-diazaspiro[5.5]undecane | | | |
| 2-hydroxy-5-{[9-(2-isobutoxybenzyl)-3,9- | D | 2.84 | 517 |
| diazaspiro[5.5]undec-3-yl]sulfonyl}benzoic acid | | | |
| methyl 3-{[9-(2-isobutoxybenzyl)-3,9- | D | 2.83 | 521 |
| diazaspiro[5.5]undec-3-yl]sulfonyl}thiophene-2- | | | |
| carboxylate | | | |
| 3-{[4-(2-furyl)phenyl]sulfonyl}-9-(2-isobutoxybenzyl)- | D | 2.8 | 524 |
| 3,9-diazaspiro[5.5]undecane | | | |
| 3-(2-isobutoxybenzyl)-9-[(4-methyl-3,4-dihydro-2H-1,4- | D | 2.89 | 528 |
| benzoxazin-7-yl)sulfonyl]-3,9-diazaspiro[5.5]undecane | | | |
| 3-(2-isobutoxybenzyl)-9-[(5-methyl-1-phenyl-1H- | D | 2.92 | 537 |
| pyrazol-4-yl)sulfonyl]-3,9-diazaspiro[5.5]undecane | | | |
| 3-(2-isobutoxybenzyl)-9-[(6-morpholin-4-ylpyridin-3- | D | 2.76 | 543 |
| yl)sulfonyl]-3,9-diazaspiro[5.5]undecane | | | |
| 3-(2,3-dihydro-1-benzofuran-5-ylsulfonyl)-9-(2- | D | 2.84 | 499 |
| isobutoxybenzyl)-3,9-diazaspiro[5.5]undecane | | | |

### EXAMPLE: 12

### 3-{[9-(2-isobutoxybenzyl)-3,9-diazaspiro[5.5]undec-3-yl]carbonyl}benzoic acid

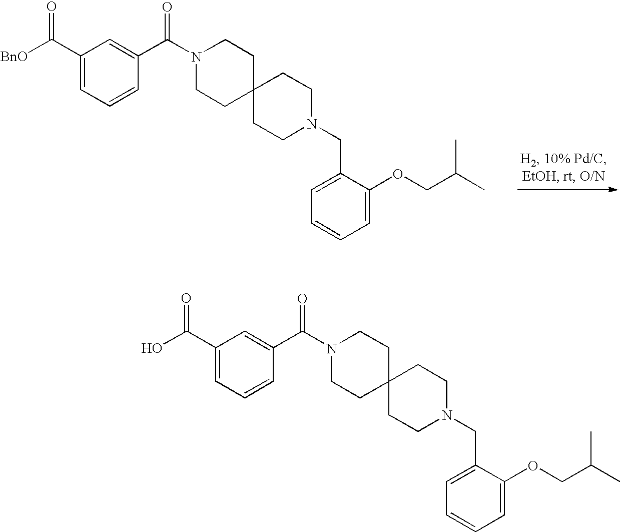

To a solution of benzyl 3-{[9-(2-isobutoxybenzyl)-3,9-diazaspiro[5.5]undec-3-yl]carbonyl}benzoate (prepared according to Example 7) (91 mg, 0.16 mmol), 10% Pd/C (10 mg), and ethanol (5 ml) was stirred under a hydrogen atmosphere until TLC indicated complete consumption of starting material. The reaction mixture was then filtered through Celite, which was then washed with ethanol, and the filtrate was concentrated to provide a crude oil. The oil was triturated with diethyl ether to provide the title compound (60 mg, 81%). LCMS (Method C): R_{T}=7.79 minutes; 465 (M+H)⁺.

The following compounds were prepared according to the general procedure used for example 12.

| | LCMS | Retention | Mass Ion/ |
|---|---|---|---|
| Compound | Method | time/min | MH⁺ |
| 4-{2-[9-(2-isobutoxybenzyl)-3,9- | C | 8.25 | 479 |
| diazaspiro[5.5]undec-3-yl]- | | | |
| 2-oxoethyl}benzoic acid | | | |
| 2-{[9-(2-isobutoxybenzyl)-3,9- | C | 7.6 | 464 |
| diazaspiro[5.5]undec-3- | | | |
| yl]carbonyl}benzoic acid | | | |
| (2-{[9-(2-isobutoxybenzyl)-3,9- | C | 7.88 | 479 |
| diazaspiro[5.5]undec-3- | | | |
| yl]carbonyl}phenyl)acetic acid | | | |

### EXAMPLE: 13

### (3-{[9-(2-isobutoxybenzyl)-3,9-diazaspiro[5.5]undec-3-yl]carbonyl}phenyl)acetic acid

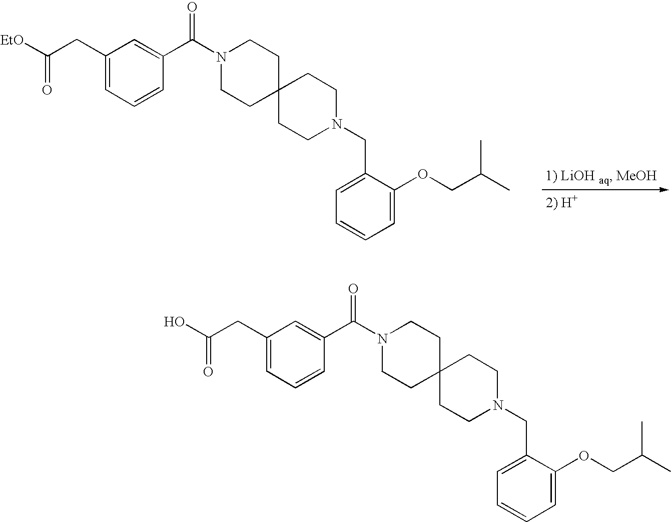

To a solution of ethyl (3-{[9-(2-isobutoxybenzyl)-3,9-diazaspiro[5.5]undec-3-yl]carbonyl}phenyl)acetate (prepared according to Example 7) (71 mg, 0.14 mmol) in methanol (3 ml) was added 1M aqueous lithium hydroxide solution (2 ml). After stirring for 2 hours the reaction mixture was concentrated to dryness to afford a viscous oil, which was triturated with diethyl ether, to provide the title compound (48 mg, 71%) as a white solid.

¹H NMR (400 MHz, DMSO-D6) δ 7.30-7.10 (m, 6H), 6.95 (t and d, 2H), 3.75 (d, 2H), 3.55 (bs, 2H), 3.45-3.20 (m, 6H), 2.35 (m, 4H), 2.00 (q, 1H), 1.50-1.30 (m, 8H), 1.00 (d, 6H); LCMS (Method C): R_{T}=7.38 minutes; 479 (M+H)⁺.

### EXAMPLE: 14

### [{2-[9-(2-isobutoxybenzyl)-3,9-diazaspiro[5.5]undec-3-yl]-2-oxoethyl}(phenyl)amino]acetic acid

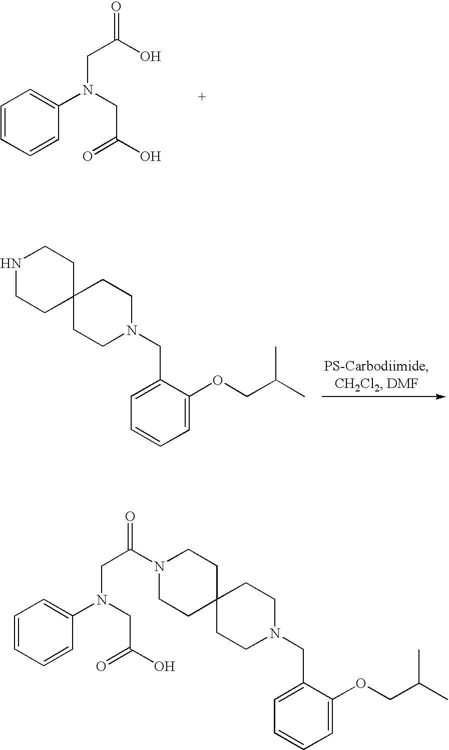

2,2′-(Phenylimino)diacetic acid (52 mg, 0.25 mmol) was dissolved in a minimal amount of N,N-dimethylformamide, then added to a slurry of polymer supported carbodiimide (250 mg, 0.3 mmol, 1.2 mmolg⁻¹ loading) and dichloromethane (3 ml). The mixture was agitated for 40 minutes before a solution of 3-(2-isobutoxybenzyl)-3,9-diazaspiro[5.5]undecane (57 mg, 0.18 mmol) and dichloromethane (1 ml) was added. The resultant mixture was agitated overnight at room temperature, then the reaction was filtered and washed with N,N-dimethylformamide, and the filtrate concentrated to provide a solid. The solid was subjected to reverse-phase preparative HPLC using acetonitrile and water (gradient 10:90 to 90:10, v/v) as eluent, to provide the title compound (56 mg, 50%).

LCMS (Method F): R_{T}=2.36 minutes; 508 (M+H)⁺.

The following compounds were prepared according to the general procedure used for example 14.

| | LCMS | Retention | Mass Ion/ |
|---|---|---|---|
| Compound | Method | time/min | MH⁺ |
| 5-{[9-(2-isobutoxybenzyl)-3,9-diazaspiro[5.5]undec-3- | F | 2.18 | 471 |
| yl]carbonyl}thiophene-2-carboxylic acid | | | |
| (2E,4E)-6-[9-(2-isobutoxybenzyl)-3,9-diazaspiro[5.5]undec- | D | 2.69 | 441 |
| 3-yl]-6-oxohexa-2,4-dienoic acid | | | |
| 6-[9-(2-isobutoxybenzyl)-3,9-diazaspiro[5.5]undec-3-yl]-6- | E | 2.23 | 445 |
| oxohexanoic acid | | | |
| 4′-{[9-(2-isobutoxybenzyl)-3,9-diazaspiro[5.5]undec-3- | F | 2.31 | 541 |
| yl]carbonyl}biphenyl-4-carboxylic acid | | | |
| (3-{2-[9-(2-isobutoxybenzyl)-3,9-diazaspiro[5.5]undec-3-yl]- | F | 2.17 | 493 |
| 2-oxoethyl}phenyl)acetic acid | | | |
| 3-{[9-(2-isobutoxybenzyl)-3,9-diazaspiro[5.5]undec-3- | E | 2.27 | 455 |
| yl]carbonyl}-1H-pyrazole-5-carboxylic acid | | | |
| {2-[9-(2-isobutoxybenzyl)-3,9-diazaspiro[5.5]undec-3-yl]-2- | D | 2.49 | 433 |
| oxoethoxy}acetic acid | | | |

### EXAMPLE: 15

The following compounds were prepared according to the general procedure described in example 3 except NaBH(OAc)₃ was used instead of NaCNBH₃ on resin and DMF instead of NMP as the solvent. The crude reaction mixture was diluted with methanol/water and loaded onto a SCX column. The column washed with MeOH and the title compound was eluted with ammonia in methanol. Some compounds were further purified with preparative HPLC to give the trifluoroacetate salt. Preparative HPLC Conditions for Example 15 were Kromasil KR-100-5-C₁₈ column (250×20 mm, Akzo Nobel) and mixtures of acetonitrile/water with 0.1% TFA at a flow rate of 10 mL/min were used for preparative HPLC.

### 3-(4-chlorobenzoyl)-9-{2-[(2,6-dichlorobenzyl)oxy]benzyl}-3,9-diazaspiro[5.5]undecane trifluoroacetate

¹H NMR (399.99 MHz, CD₃OD) δ 7.61-7.35 (m, 9H), 7.14 (t, J=7.4 Hz, 1H), 5.45 (s, 2H), 4.26 (s, 2H), 3.78-3.61 (m, 2H), 3.44-3.30 (m, 16H), 3.19-3.00 (m, 2H), 1.94 (d, J=14.4 Hz, 2H), 1.68-1.36 (m, 6H)

LC-MS: m/z 557 [MH+]

### 3-(4-chlorobenzoyl)-9-[2-(2-methoxyphenoxy)benzyl]-3,9-diazaspiro[5.5]undecane trifluoroacetate

¹H NMR (399.99 MHz, CD₃OD) δ 7.55-7.26 (m, 7H), 7.21-7.01 (m, 4H), 6.61 (d, J=9.0 Hz, 1H), 4.53 (s, 2H), 3.75 (s, 5H), 3.56-3.48 (m, 2H), 3.47-3.39 (m, 2H), 3.31-3.22 (m, 2H), 2.06 (d, J=13.9 Hz, 2H), 1.87-1.40 (m, 6H)

LC-MS: m/z 505 [MH+]

### 3-[2-(tert-butylthio)benzyl]-9-(4-chlorobenzoyl)-3,9-diazaspiro[5.5]undecane trifluoroacetate

¹H NMR (399.99 MHz, CD₃OD) δ 7.80-7.77 (m, 1H), 7.68 (d, J=7.0 Hz, 1H), 7.61-7.52 (m, 2H), 7.48 (d, J=8.2 Hz, 2H), 7.41 (d, J=8.4 Hz, 2H), 4.68 (s, 2H), 3.81-3.68 (m, 2H), 3.50-3.20 (m, 6H), 2.02 (d, J=14.8 Hz, 2H), 1.87-1.38 (m, 6H), 1.30 (s, 9H)

LC-MS: m/z 471 [MH+]

### 3-(4-chlorobenzoyl)-9-[3-(pyridin-2-yloxy)benzyl]-3,9-diazaspiro[5.5]undecane trifluoroacetate

¹H NMR (399.99 MHz, CD₃OD) δ 8.13 (d, J=4.9 Hz, 1H), 7.88 (dt, 1H), 7.55 (t, J=7.7 Hz, 2H), 7.48 (d, J=8.6 Hz, 3H), 7.40 (d, J=8.1 Hz, 4H), 7.35 (d, J=7.3 Hz, 4H), 7.31 (s, 3H), 7.25 (d, J=8.1 Hz, 2H), 7.16 (dd, 1H), 7.06 (d, J=8.6 Hz, 1H), 4.33 (s, 3H), 3.80-3.67 (m, 3H), 3.50-3.34 (m, 10H), 3.26-3.06 (m, 6H), 2.03 (d, J=14.9 Hz, 3H), 1.86-1.37 (m, 8H)

LC-MS: m/z 476 [MH+]

### 3-(4-chlorobenzoyl)-9-[(3,5-diethoxypyridin-4-yl)methyl]-3,9-diazaspiro[5.5]undecane trifluoroacetate

¹H NMR (399.99 MHz, CD₃OD) δ 8.16 (s, 2H), 7.48 (d, J=8.9 Hz, 2H), 7.41 (d, J=8.8 Hz, 2H), 4.41 (s, 2H), 4.31 (q, J=6.9 Hz, 4H), 3.82-3.68 (m, 2H), 3.51-3.39 (m, 4H), 3.35-3.24 (m, 2H), 2.10-1.99 (m, 2H), 1.85-1.53 (m, 6H), 1.50 (t, J=7.2 Hz, 6H)

LC-MS: m/z 472 [MH+]

### 2-(2-{[9-(4-chlorobenzoyl)-3,9-diazaspiro[5.5]undec-3-yl]methyl}phenoxy)benzonitrile

¹H NMR (399.99 MHz, CD₃OD) δ 7.74 (dd, 1H), 7.56-7.50 (m, 2H), 7.48-7.35 (m, 5H), 7.28 (t, J=7.5 Hz, 1H), 7.18 (t, J=7.7 Hz, 1H), 6.77 (d, J=8.5 Hz, 1H), 7.06 (d, J=8.0 Hz, 1H), 3.72-3.62 (m, 2H), 3.58 (s, 2H), 3.38-3.31 (m, 2H), 2.54-2.39 (m, 4H), 1.56-1.33 (m, 8H)

LC-MS: m/z 500 [MH+]

### EXAMPLE: 16

The following compounds were prepared according to the general procedure described in example 1 except DMF was used instead of NMP as the solvent. The crude reaction mixture was diluted with methanol/water and loaded onto a SCX column. The column was washed with MeOH and the title compound was eluted with ammonia in methanol. Some compounds were further purified with preparative HPLC to give the trifluoroacetate salt. Preparative HPLC conditions for Example 16, where used, were Kromasil KR-100-5-C₁₈ column (250×20 mm, Akzo Nobel) and mixtures of acetonitrile/water with 0.1% TFA at a flow rate of 10 mL/min were used for preparative HPLC.

### 3-[2-(allyloxy)benzyl]-9-(4-chlorobenzoyl)-3,9-diazaspiro[5.5]undecane

LC-MS (Method A) RT: 3.97 min, m/z 439 [MH+]

### 3-[3-(benzyloxy)benzyl]-9-(4-chlorobenzoyl)-3,9-diazaspiro[5.5]undecane

LC-MS (Method A) RT: 4.86 min, m/z 489 [MH+]

### 3-(4-chlorobenzoyl)-9-(4-phenoxybenzyl)-3,9-diazaspiro[5.5]undecane

LC-MS (Method A) RT: 4.78 min, m/z 475 [MH+]

### 3-(4-chlorobenzoyl)-9-[2-(4-methylphenoxy)benzyl]-3,9-diazaspiro[5.5]undecane

LC-MS (Method A) RT: 4.97 min, m/z 489 [MH+]

### 3-[2-(4-tert-butylphenoxy)benzyl]-9-(4-chlorobenzoyl)-3,9-diazaspiro[5.5]undecane

LC-MS (Method A) RT: 5.63 min, m/z 531 [MH+]

### 3-(4-chlorobenzoyl)-9-[2-(3-chlorophenoxy)benzyl]-3,9-diazaspiro[5.5]undecane

LC-MS (Method A) RT: 4.97 min, m/z 509 [MH+]

### 3-(4-chlorobenzoyl)-9-[2-(4-fluorophenoxy)benzyl]-3,9-diazaspiro[5.5]undecane

LC-MS (Method A) RT: 4.77 min, m/z 493 [MH+]

### 3-(4-chlorobenzoyl)-9-{2-[3-(trifluoromethyl)phenoxy]benzyl}-3,9-diazaspiro[5.5]undecane

LC-MS (Method A) RT: 5.14 min, m/z 543 [MH+]

### 3-(4-chlorobenzoyl)-9-[2-(2,4-dichlorophenoxy)benzyl]-3,9-diazaspiro[5.5]undecane

LC-MS (Method A) RT: 5.18 min, m/z 543 [MH+]

### 3-(4-chlorobenzoyl)-9-{2-[(2-fluorophenyl)thio]benzyl}-3,9-diazaspiro[5.5]undecane

LC-MS (Method A) RT: 4.86 min, m/z 509 [MH+]

### 3-(4-chlorobenzoyl)-9-(4-fluoro-3-phenoxybenzyl)-3,9-diazaspiro[5.5]undecane

LC-MS (Method A) RT: 4.82 min, m/z 493 [MH+]

### 3-(4-chlorobenzoyl)-9-(2-isobutoxybenzyl)-3,9-diazaspiro[5.5]undecane

LC-MS (Method A) RT: 4.80 min, m/z 455 [MH+]

### 2-[2-(allyloxy)benzyl]-7-(4-chlorobenzoyl)-2,7-diazaspiro[3.5]nonane trifluoroacetate

LC-MS (Method A) RT: 4.24 min, m/z 411 [MH+]

### 7-(4-chlorobenzoyl)-2-[2-(3-chlorophenoxy)benzyl]-2,7-diazaspiro[3.5]nonane trifluoroacetate

LC-MS (Method A) RT: 4.91 min, m/z 481 [MH+]

### 7-(4-chlorobenzoyl)-2-[2-(4-fluorophenoxy)benzyl]-2,7-diazaspiro[3.5]nonane trifluoroacetate

LC-MS (Method A) RT: 4.72 min. m/z 465 [MH+]

### 7-(4-chlorobenzoyl)-2-{2-[3-(trifluoromethyl)phenoxy]benzyl}-2,7-diazaspiro[3.5]nonane trifluoroacetate

LC-MS (Method A) RT: 5.07 min, m/z 515 [MH+]

### 2-(2-{[7-(4-chlorobenzoyl)-2,7-diazaspiro[3.5]non-2-yl]methyl}phenoxy)benzonitrile trifluoroacetate

LC-MS (Method A) RT: 4.44 min, m/z 472 [MH+]

### 7-(4-chlorobenzoyl)-2-[2-(pyridin-3-yloxy)benzyl]-2,7-diazaspiro[3.5]nonane trifluoroacetate

LC-MS (Method A) RT: 3.25 min, m/z 448 [MH+]

### 7-(4-chlorobenzoyl)-2-(4-fluoro-3-phenoxybenzyl)-2,7-diazaspiro[3.5]nonane trifluoroacetate

LC-MS (Method A) RT: 4.93 min, m/z 465 [MH+]

### 7-(4-chlorobenzoyl)-2-(2-isobutoxybenzyl)-2,7-diazaspiro[3.5]nonane trifluoroacetate

LC-MS (Method A) RT: 4.56 nm in, m/z 427 [MH+]

### 7-[2-(allyloxy)benzyl]-2-(4-chlorobenzoyl)-2,7-diazaspiro[3.5]nonane trifluoroacetate

LC-MS (Method A) RT: 4.21 min, m/z 411 [MH+]

### 7-[3-(benzyloxy)benzyl]-2-(4-chlorobenzoyl)-2,7-diazaspiro[3.5]nonane trifluoroacetate

LC-MS (Method A) RT: 4.63 min, m/z 461 [MH+]

### 2-(4-chlorobenzoyl)-7-[2-(3-chlorophenoxy)benzyl]-2,7-diazaspiro[3.5]nonane trifluoroacetate

LC-MS (Method A) RT: 4.93 min, m/z 481 [MH+]

### 2-(4-chlorobenzoyl)-7-[2-(4-fluorophenoxy)benzyl]-2,7-diazaspiro[3.5]nonane trifluoroacetate

LC-MS (Method A) RT: 4.68 min, m/z 465 [MH+]

### 2-(4-chlorobenzoyl)-7-{2-[3-(trifluoromethyl)phenoxy]benzyl}-2,7-diazaspiro[3.5]nonane trifluoroacetate

LC-MS (Method A) RT: 5.08 min, m/z 515 [MH+]

### 2-(2-{[2-(4-chlorobenzoyl)-2,7-diazaspiro[3.5]non-7-yl]methyl}phenoxy)benzonitrile trifluoroacetate

LC-MS (Method A) RT: 4.41 min, m/z 472 [MH+]

### 2-(4-chlorobenzoyl)-7-[2-(pyridin-3-yloxy)benzyl]-2,7-diazaspiro[3.5]nonane trifluoroacetate

LC-MS (Method A) RT: 3.24 min, m/z 448 [MH+]

### 2-(4-chlorobenzoyl)-7-(4-fluoro-3-phenoxybenzyl)-2,7-diazaspiro[3.5]nonane trifluoroacetate

LC-MS (Method A) RT: 4.71 min, m/z 465 [MH+]

### 2-(4-chlorobenzoyl)-7-(2-isobutoxybenzyl)-2,7-diazaspiro[3.5]nonane trifluoroacetate

LC-MS (Method A) RT: 4.69 min, m/z 427 [MH+]

### 8-[2-(allyloxy)benzyl]-2-(4-chlorobenzoyl)-2,8-diazaspiro[4.5]decane

LC-MS (Method A) RT: 4.25 min, m/z 425 [MH+]

### 8-[3-(benzyloxy)benzyl]-2-(4-chlorobenzoyl)-2,8-diazaspiro[4.5]decane

LC-MS (Method A) RT: 4.81 min, m/z 475 [MH+]

### 2-(4-chlorobenzoyl)-8-(4-phenoxybenzyl)-2,8-diazaspiro[4.5]decane

LC-MS (Method A) RT: 4.71 min, m/z 461 [MH+]

### 2-(4-chlorobenzoyl)-8-[2-(3-chlorophenoxy)benzyl]-2,8-diazaspiro[4.5]decane

LC-MS (Method A) RT: 4.93 min, m/z 495 [MH+]

### 2-(4-chlorobenzoyl)-8-[2-(4-fluorophenoxy)benzyl]-2,8-diazaspiro[4.5]decane

LC-MS (Method A) RT: 4.72 min, m/z 479 [MH+]

### 2-(4-chlorobenzoyl)-8-{2-[3-(trifluoromethyl)phenoxy]benzyl}-2,8-diazaspiro[4.5]decane

LC-MS (Method A) RT: 5.07 min, m/z 529 [MH+]

### 2-(4-chlorobenzoyl)-8-[2-(2,4-dichlorophenoxy)benzyl]-2,8-diazaspiro[4.5]decane

LC-MS (Method A) RT: 5.11 min, m/z 529 [MH+]

### 2-(2-{[2-(4-chlorobenzoyl)-2,8-diazaspiro[4.5]dec-8-yl]methyl}phenoxy)benzonitrile

LC-MS (Method A) RT: 4.40 min, m/z 486 [MH+]

### 2-(4-chlorobenzoyl)-8-(4-fluoro-3-phenoxybenzyl)-2,8-diazaspiro[4.5]decane

LC-MS (Method A) RT: 4.71 min, m/z 479 [MH+]

### 2-(4-chlorobenzoyl)-8-(2-isobutoxybenzyl)-2,8-diazaspiro[4.5]decane

LC-MS (Method A) RT: 4.62 min, m/z 441 [MH+]

### 2-[2-(allyloxy)benzyl]-8-(4-chlorobenzoyl)-2,8-diazaspiro[4.5]decane

LC-MS (Method A) RT: 4.15 min, m/z 425 [MH+]

### 2-[3-(benzyloxy)benzyl]-8-(4-chlorobenzoyl)-2,8-diazaspiro[4.5]decane

LC-MS (Method A) RT: 4.82 min, m/z 475 [MH+]

### 8-(4-chlorobenzoyl)-2-[2-(3-chlorophenoxy)benzyl]-2,8-diazaspiro[4.5]decane

LC-MS (Method A) RT: 4.98 min, m/z 495 [MH+]

### 8-(4-chlorobenzoyl)-2-[2-(4-fluorophenoxy)benzyl]-2,8-diazaspiro[4.5]decane

LC-MS (Method A) RT: 4.75 min, m/z 479 [MH+]

### 8-(4-chlorobenzoyl)-2-{2-[3-(trifluoromethyl)phenoxy]benzyl}-2,8-diazaspiro[4.5]decane

LC-MS (Method A) RT: 5.11 min, m/z 529 [MH+]

### 2-(2-{[8-(4-chlorobenzoyl)-2,8-diazaspiro[4.5]dec-2-yl]methyl}phenoxy)benzonitrile

LC-MS (Method A) RT: 4.52 min, m/z 486 [MH+]

### 8-(4-chlorobenzoyl)-2-{2-[(2-chloro-1,3-thiazol-5-yl)methoxy]benzyl}-2,8-diazaspiro[4.5]decane

LC-MS (Method A) RT: 4.43 min, m/z 516 [MH+]

### 8-(4-chlorobenzoyl)-2-(4-fluoro-3-phenoxybenzyl)-2,8-diazaspiro[4.5]decane

LC-MS (Method A) RT: 4.77 min, m/z 479 [MH+]

### 8-(4-chlorobenzoyl)-2-(2-isobutoxybenzyl)-2,8-diazaspiro[4.5]decane

LC-MS (Method A) RT: 4.75 min, m/z 441 [MH+]

### EXAMPLE 17

The following compounds were prepared according to the general procedure used for example 2b, except that the solvent was DMF instead of NMP.

### 3-(4-chlorobenzoyl)-9-[2-(3-methylbutoxy)benzyl]-3,9-diazaspiro[5.5]undecane trifluoroacetate

¹H NMR (299.945 MHz, CD₃OD) δ 7.51-7.39 (m, 6H), 7.14 (d, J=8.2 Hz, 1H), 7.05 (t, J=7.5 Hz, 1H), 3.75 (s, 2H), 3.42-3.23 (m, 8H), 2.03 (d, J=14.6 Hz, 2H), 1.90-1.45 (m, 9H), 1.01 (d, J=6.2 Hz, 6H)

APCI-MS m/z: 469/471 (3:1) [MH+]

### 3-benzoyl-9-[2-(3-methylbutoxy)benzyl]-3,9-diazaspiro[5.5]undecane trifluoroacetate

¹H NMR (299.945 MHz, CD₃OD) δ 7.51-7.38 (m, 7H), 7.14 (d, J=8.2 Hz, 1H), 7.05 (t, J=7.4 Hz, 1H), 4.33 (s, 2H), 4.15 (t, J=6.7 Hz, 2H), 3.72-3.24 (m, 8H), 2.03 (d, J=14.8 Hz, 2H), 1.88-1.44 (m, 9H), 1.01 (d, J=6.0 Hz, 6H)

APCI-MS m/z: 435 [MH+]

### 3-(2-ethoxybenzyl)-9-(4-fluorobenzoyl)-3,9-diazaspiro[5.5]undecane trifluoroacetate

¹H NMR (299.945 MHz, CD₃OD) δ 7.53-7.40 (m, 4H), 7.55-7.01 (m, 4H), 4.34 (s, 2H), 4.19 (q, 2H), 3.72-3.24 (m, 8H), 2.07-1.59 (m, 8H), 1.46 (t, 3H)

APCI-MS m/z: 411 [MH+]

### 3-(2-ethoxybenzyl)-9-(4-nitrobenzoyl)-3,9-diazaspiro[5.5]undecane trifluoroacetate

¹H NMR (299.945 MHz, CD₃OD) δ 8.33 (d, J=8.4 Hz, 2H), 7.65 (d, J=8.2 Hz, 2H), 7.50-7.42 (m, 2H), 7.12 (d, J=8.4 Hz, 1H), 7.04 (t, J=7.4 Hz, 1H), 4.34 (d, J=8.2 Hz, 2H), 4.20 (q, J=13.3 Hz, 2H), 3.79-3.19 (m, 8H), 2.07-1.59 (m, 8H), 1.47 (t, J=5.9 Hz, 3H)

APCI-MS m/z: 438 [MH+]

### 3-(4-chlorobenzoyl)-9-(2-isobutoxybenzyl)-3,9-diazaspiro[5.5]undecane trifluoroacetate

¹H NMR (299.945 MHz, CD₃OD) δ 7.47-7.39 (m, 6H), 7.13 (d, J=21.0 Hz, 1H), 7.05 (t, J=12.5 Hz, 1H), 4.35 (s, 2H), 3.89 (d, J=6.6 Hz, 2H), 3.81-3.08 (m, 8H), 2.10-2.22 (m, 1H), 2.04 (d, J=14.5 Hz, 2H), 1.78-1.45 (m, 6H), 1.09 (d, J=6.6 Hz, 6H)

APCI-MS m/z: 455 [MH+]

### 3-(2-isobutoxybenzyl)-9-(4-nitrobenzoyl)-3,9-diazaspiro[5.5]undecane trifluoroacetate

¹H NMR (299.945 MHz, CD₃OD) δ 8.33 (d, J=8.4 Hz, 2H), 7.65 (d, J=8.1 Hz, 2H), 7.51-7.42 (m, 2H), 7.13 (d, J=8.6 Hz, 1H), 7.05 (t, J=7.4 Hz, 1H), 4.35 (d, J=7.9 Hz, 2H), 3.89 (d, J=4.8 Hz, 2H), 3.78-3.20 (m, 8H), 2.22-2.11 (m, 1H), 2.05 (d, J=14.5 Hz, 2H), 1.82-1.45 (m, 6H), 1.09 (t, J=6.3 Hz, 6H)

APCI-MS m/z: 466 [MH+]

### 3-(4-fluorobenzoyl)-9-(2-isobutoxybenzyl)-3,9-diazaspiro[5.5]undecane trifluoroacetate

¹H NMR (299.945 MHz, CD₃OD) δ 7.51-7.42 (m, 4H), 7.20 (t, J=8.8 Hz, 2H), 7.13 (d, J=8.2 Hz, 1H), 7.05 (t, J=7.4 Hz, 1H), 4.35 (s, 2H), 3.89 (d, J=6.4 Hz, 2H), 3.78-3.16 (m, 8H), 2.25-2.10 (m, 1H), 2.04 (d, J=15.4 Hz, 2H), 1.85-1.42 (m, 6H), 1.09 (d, J=6.8 Hz, 6H)

APCI-MS m/z: 439 [MH+]

### 2-chloro-5-{[9-(2-isobutoxybenzyl)-3,9-diazaspiro[5.5]undec-3-yl]carbonyl}benzenesulfonamide trifluoroacetate

¹H NMR (299.945 MHz, CD₃OD) δ 8.08 (s, 1H), 7.70 (d, J=8.1 Hz, 1H), 7.60 (dd, J=8.1, 1.7 Hz, 1H), 7.51-7.42 (m, 2H), 7.13 (d, J=8.2 Hz, 1H), 7.05 (t, J=7.5 Hz, 1H), 4.35 (s, 2H), 3.90 (d, J=31.3 Hz, 2H), 3.79-3.18 (m, 8H), 2.26-2.10 (m, 1H), 2.05 (d, J=14.8 Hz, 2H), 1.89-1.37 (m, 6H), 1.09 (d, J=6.8 Hz, 6H)

APCI-MS m/z: 534/536 (3:1) [MH+]

### 3-(2-isobutoxybenzyl)-9-(1H-pyrrol-3-ylcarbonyl)-3,9-diazaspiro[5.5]undecane trifluoroacetate

¹H NMR (299.945 MHz, CD₃OD) δ 7.53-7.43 (m, 2H), 7.14 (d, J=10.2 Hz, 1H), 7.06 (t, J=7.2 Hz, 1H), 6.92 (d, J=1.5 Hz, 1H), 6.56 (t, J=1.9 Hz, 1H), 6.20 (d, J=2.6 Hz, 1H), 4.36 (s, 2H), 3.90 (d, J=6.4 Hz, 2H), 3.81 (s, 4H), 3.47-3.19 (m, 4H), 2.22-2.13 (m, 1H), 2.05 (d, J=16.5 Hz, 2H), 1.78-1.51 (m, 6H), 1.10 (dd, J=6.7, 5.0 Hz, 6H)

APCI-MS m/z: 534 [MH+]

### 8-(2-isobutoxybenzyl)-2-[2-(methylsulfonyl)benzoyl]-2,8-diazaspiro[4.5]decane trifluoroacetate

¹H NMR (299.945 MHz, CD₃OD) δ 8.12-8.06 (1H), 7.85-7.69 (2H), 7.56-7.35 (3H), 7.16-6.98 (2H), 4.39-4.23 (2H), 3.92-3.83 (2H), 3.75-3.64 (1H), 3.55-2.95 (7H), 3.27 (3H), 2.24-1.85 (7H), 1.13-1.01 (6H)

APCI-MS m/z: 485 [MH+]

### 2-[4-chloro-2-(methylsulfonyl)benzoyl]-8-(2-isobutoxybenzyl)-2,8-diazaspiro[4.5]decane trifluoroacetate

¹H NMR (299.945 MHz, CD₃OD) δ 8.10-8.06 (1H), 7.87-7.80 (1H), 7.58-7.34 (3H), 7.15-6.98 (2H), 4.38-4.23 (2H), 3.92-3.83 (2H), 3.74-2.93 (1H), 2.19-1.86 (7H), 1.12-1.02 (6H)

APCI-MS m/z: 519/521 (3:1) [MH+]

### 2-{[8-(2-isobutoxybenzyl)-2,8-diazaspiro[4.5]dec-2-yl]carbonyl}nicotinamide trifluoroacetate

¹H NMR (299.945 MHz, cd3od) δ 8.71-8.64 (1H), 8.24-8.15 (1H), 7.64-7.35 (3H), 7.20-6.99 (2H), 4.43-4.18 (2H), 3.95-3.79 (2H), 3.58-2.90 (10H), 2.27-1.67 (7H), 1.16-0.96 (6H)

APCI-MS m/z: 451 [MH+]

### 8-(2-isobutoxybenzyl)-2-[(2-morpholin-4-ylpyridin-3-yl)carbonyl]-2,8-diazaspiro[4.5]decane trifluoroacetate

¹H NMR (299.945 MHz, CD₃OD) δ 8.35-8.21 (1H), 7.70-7.61 (1H), 7.53-7.35 (2H), 7.20-6.90 (3H), 4.39-4.21 (2H), 3.92-3.85 (2H), 3.77-3.71 (4H), 3.71-3.39 (4H), 3.39-3.34 (4H), 3.28-2.91 (4H), 2.21-1.85 (7H), 1.12-1.03 (6H)

APCI-MS m/z: 493 [MH+]

### 2-[(2,6-dimethoxypyridin-3-yl)carbonyl]-8-(2-isobutoxybenzyl)-2,8-diazaspiro[4.5]decane trifluoroacetate

¹H NMR (299.945 MHz, CD₃OD) δ 7.63-7.57 (1H), 7.52-7.37 (2H), 7.15-7.01 (2H), 6.45-6.38 (1H), 4.38-4.28 (2H), 4.01-3.92 (6H), 3.92-3.85 (2H), 3.72-3.01 (8H), 2.23-1.73 (7H), 1.12-1.03 (6H)

APCI-MS m/z: 468 [MH+]

### 2-(2,4-dimethoxybenzoyl)-8-(2-isobutoxybenzyl)-2,8-diazaspiro[4.5]decane trifluoroacetate

¹H NMR (299.945 MHz, CD₃OD) δ 7.52-7.37 (2H), 7.20-7.01 (3H), 6.65-6.56 (2H), 4.39-4.28 (2H), 3.92-3.82 (2H), 3.83 (3H), 3.83 (3H), 3.71-3.35 (5H), 3.26-2.91 (3H), 2.21-1.70 (7H), 1.12-1.03 (6H),

APCI-MS m/z: 467 [MH+]

### EXAMPLE 18

The following compound was prepared according to the general procedure used for example 6.

### 3-[(4-chlorobenzyl)sulfonyl]-9-(2-ethoxybenzyl)-3,9-diazaspiro[5.5]undecane trifluoroacetate

¹H NMR (299.945 MHz, CD₃OD) δ 7.51-7.38 (m, 6H), 7.12 (d, J=8.4 Hz, 1H), 7.04 (t, J=7.5 Hz, 1H), 4.33 (s, 4H), 4.18 (q, J=7.0 Hz, 2H), 3.40-3.16 (m, 8H), 1.92 (d, J=14.3 Hz, 2H), 1.74-1.52 (m, 6H), 1.47 (t, J=7.0 Hz, 3H)

APCI-MS m/z: 477/479 (3:1) [MH+]

### EXAMPLE 19

The following compounds were prepared according to the general procedure used for example 2.

### 8-(2-isobutoxybenzyl)-2-[4-(methylsulfonyl)benzoyl]-2,8-diazaspiro[4.5]decane trifluoroacetate

LC-MS (Method A) RT: 4.05 min, m/z 485.3 [MH+]

### 8-(2-isobutoxybenzyl)-2-[2-methoxy-4-(methylthio)benzoyl]-2,8-diazaspiro[4.5]decane trifluoroacetate

LC-MS (Method A) RT: 4.65 min, m/z 483.3 [MH+]

### 2-(4-butoxybenzoyl)-8-(2-isobutoxybenzyl)-2,8-diazaspiro[4.5]decane trifluoroacetate

LC-MS (Method A) RT: 5.20 min, m/z 479.4 [MH+]

### 1-(4-{[8-(2-isobutoxybenzyl)-2,8-diazaspiro[4.5]dec-2-yl]carbonyl}phenyl)ethanone trifluoroacetate

LC-MS (Method A) RT: 4.19 min, m/z 449.3 [MH+]

### 2-(4-ethylbenzoyl)-8-(2-isobutoxybenzyl)-2,8-diazaspiro[4.5]decane trifluoroacetate

LC-MS (Method A) RT: 4.78 min, m/z 435.3 [MH+]

### 2-{[8-(2-isobutoxybenzyl)-2,8-diazaspiro[4.5]dec-2-yl]carbonyl}quinoline bis(trifluoroacetate)

LC-MS (Method A) RT: 4.53 min, m/z 458.3 [MH+]

### 2-(4-chloro-2-methoxybenzoyl)-8-(2-isobutoxybenzyl)-2,8-diazaspiro[4.5]decane trifluoroacetate

LC-MS (Method A) RT: 4.72 min, m/z 471.3 [MH+]

### 8-(2-isobutoxybenzyl)-2-(4-morpholin-4-ylbenzoyl)-2,8-diazaspiro[4.5]decane bis(trifluoroacetate)

LC-MS (Method A) RT: 4.19 min, m/z 492.4 [MH+]

### 8-(2-isobutoxybenzyl)-2-(6-methoxy-2-naphthoyl)-2,8-diazaspiro[4.5]decane trifluoroacetate

LC-MS (Method A) RT: 4.84 min, m/z 487.3 [MH+]

### 2-(2,3-dichlorobenzoyl)-8-(2-isobutoxybenzyl)-2,8-diazaspiro[4.5]decane trifluoroacetate

LC-MS (Method A) RT: 4.83 min, m/z 475.2 [MH+]

### 8-(2-isobutoxybenzyl)-2-(3-methoxybenzoyl)-2,8-diazaspiro[4.5]decane trifluoroacetate

LC-MS (Method A) RT: 4.35 min, m/z 437.3 [MH+]

### 2-(2,3-dimethylbenzoyl)-8-(2-isobutoxybenzyl)-2,8-diazaspiro[4.5]decane trifluoroacetate

LC-MS (Method A) RT: 4.67 min, m/z 435.3 [MH+]

### 8-(2-isobutoxybenzyl)-2-(4-methylbenzoyl)-2,8-diazaspiro[4.5]decane trifluoroacetate

LC-MS (Method A) RT: 4.49 min. m/z 421.3 [MH+]

### 2-(3,4-dichlorobenzoyl)-8-(2-isobutoxybenzyl)-2,8-diazaspiro[4.5]decane trifluoroacetate

LC-MS (Method A) RT: 4.90 min, m/z 475.2 [MH+]

### 2-(2,4-dichlorobenzoyl)-8-(2-isobutoxybenzyl)-2,8-diazaspiro[4.5]decane trifluoroacetate

LC-MS (Method A) RT: 4.88 min, m/z 475.2 [MH+]

### 8-(2-isobutoxybenzyl)-2-(4-isopropoxybenzoyl)-2,8-diazaspiro[4.5]decane trifluoroacetate

LC-MS (Method A) RT: 4.79 min, m/z 465.3 [MH+]

### 8-(2-isobutoxybenzyl)-2-(4-phenoxybenzoyl)-2,8-diazaspiro[4.5]decane trifluoroacetate

LC-MS (Method A) RT: 5.11 min, m/z 499.3 [MH+]

### 8-(2-isobutoxybenzyl)-2-(2-naphthoyl)-2,8-diazaspiro[4.5]decane trifluoroacetate

LC-MS (Method A) RT: 4.76 min, m/z 457.3 [MH+]

### 2-(2-chlorobenzoyl)-8-(2-isobutoxybenzyl)-2,8-diazaspiro[4.5]decane trifluoroacetate

LC-MS (Method A) RT: 4.53 min, m/z 441.3 [MH+]

### 2-(2,3-dimethoxybenzoyl)-8-(2-isobutoxybenzyl)-2,8-diazaspiro[4.5]decane trifluoroacetate

LC-MS (Method A) RT: 4.42 min, m/z 467.3 [MH+]

### 8-(2-isobutoxybenzyl)-2-(1-naphthoyl)-2,8-diazaspiro[4.5]decane trifluoroacetate

LC-MS (Method A) RT: 4.74 min, m/z 457.3 [MH+]

### 8-(2-isobutoxybenzyl)-2-(4-methoxybenzoyl)-2,8-diazaspiro[4.5]decane trifluoroacetate

LC-MS (Method A) RT: 4.33 min, m/z 437.3 [MH+]

### N,N-diethyl-4-{[8-(2-isobutoxybenzyl)-2,8-diazaspiro[4.5]dec-2-yl]carbonyl}aniline bis(trifluoroacetate)

LC-MS (Method A) RT: 3.70 min, m/z 478.3 [MH+]

### 8-(2-isobutoxybenzyl)-2-(4-propylbenzoyl)-2,8-diazaspiro[4.5]decane trifluoroacetate

LC-MS (Method A) RT: 5.03 min, m/z 449.3 [MH+]

### 8-(2-isobutoxybenzyl)-2-[3-(trifluoromethyl)benzoyl]-2,8-diazaspiro[4.5]decane trifluoroacetate

LC-MS (Method A) RT: 4.85 min, m/z 475.3 [MH+]

### 8-(2-isobutoxybenzyl)-2-[4-(trifluoromethyl)benzoyl]-2,8-diazaspiro[4.5]decane trifluoroacetate

LC-MS (Method A) RT: 4.88 min, m/z 475.3 [MH+]

### 4-{[8-(2-isobutoxybenzyl)-2,8-diazaspiro[4.5]dec-2-yl]carbonyl}quinoline bis(trifluoroacetate)

LC-MS (Method A) RT: 3.65 min, m/z 458.3 [MH+]

### 2-(3-chloro-2-methylbenzoyl)-8-(2-isobutoxybenzyl)-2,8-diazaspiro[4.5]decane trifluoroacetate

LC-MS (Method A) RT: 4.82 min, m/z 455.3 [MH+]

### (4-{2-[8-(2-isobutoxybenzyl)-2,8-diazaspiro[4.5]dec-2-yl]-2-oxoethyl}phenyl)dimethylamine bis(trifluoroacetate)

LC-MS (Method A) RT: 3.54 min, m/z 464.4 [MH+]

### 2-[(2-fluorophenyl)acetyl]-8-(2-isobutoxybenzyl)-2,8-diazaspiro[4.5]decane trifluoroacetate

LC-MS (Method A) RT: 4.53 min, m/z 439.3 [MH+]

### 8-(2-isobutoxybenzyl)-2-[(3-nitrophenyl)acetyl]-2,8-diazaspiro[4.5]decane trifluoroacetate

LC-MS (Method A) RT: 4.55 min, m/z 466.3 [MH+]

### 8-(2-isobutoxybenzyl)-2-[(4-nitrophenyl)acetyl]-2,8-diazaspiro[4.5]decane trifluoroacetate

LC-MS (Method A) RT: 4.57 min, m/z 466.3 [MH+]

### 8-(2-isobutoxybenzyl)-2-[(2-nitrophenyl)acetyl]-2,8-diazaspiro[4.5]decane trifluoroacetate

LC-MS (Method A) RT: 4.52 min, m/z 466.3 [MH+]

### 2-[(3,4-dimethoxyphenyl)acetyl]-8-(2-isobutoxybenzyl)-2,8-diazaspiro[4.5]decane trifluoroacetate

LC-MS (Method A) RT: 4.26 min, m/z 481.3 [MH+]

### 2-(3-furoyl)-8-(2-isobutoxybenzyl)-2,8-diazaspiro[4.5]decane trifluoroacetate

LC-MS (Method A) RT: 4.06 min, m/z 397.3 [MH+]

### 2-[(4-chlorophenyl)acetyl]-8-(2-isobutoxybenzyl)-2,8-diazaspiro[4.5]decane trifluoroacetate

LC-MS (Method A) RT: 4.79 min, m/z 455.3 [MH+]

### 8-(2-isobutoxybenzyl)-2-(1,2,3-thiadiazol-4-ylcarbonyl)-2,8-diazaspiro[4.5]decane bis(trifluoroacetate)

LC-MS (Method A) RT: 4.01 min, m/z 415.3 [MH+]

### 8-(2-isobutoxybenzyl)-2-[(5-methyl-1H-pyrazol-3-yl)carbonyl]-2,8-diazaspiro[4.5]decane bis(trifluoroacetate)

LC-MS (Method A) RT: 3.85 min, m/z 411.3 [MH+]

### 2-[(1,5-dimethyl-1H-pyrazol-3-yl)carbonyl]-8-(2-isobutoxybenzyl)-2,8-diazaspiro[4.5]decane bis(trifluoroacetate)

LC-MS (Method A) RT: 4.00 min, m/z 425.4 [MH+]

### 2-[(4-butoxyphenyl)acetyl]-8-(2-isobutoxybenzyl)-2,8-diazaspiro[4.5]decane trifluoroacetate

LC-MS (Method A) RT: 5.26 min, m/z 493.4 [MH+]

### 2-[(3,5-difluorophenyl)acetyl]-8-(2-isobutoxybenzyl)-2,8-diazaspiro[4.5]decane trifluoroacetate

LC-MS (Method A) RT: 4.69 min, m/z 457.3 [MH+]

### 2-[(2,4-dichlorophenyl)acetyl]-8-(2-isobutoxybenzyl)-2,8-diazaspiro[4.5]decane trifluoroacetate

LC-MS (Method A) RT: 5.00 min, m/z 489.2 [MH+]

### 2-[(2,4-difluorophenyl)acetyl]-8-(2-isobutoxybenzyl)-2,8-diazaspiro[4.5]decane trifluoroacetate

LC-MS (Method A) RT: 4.67 min, m/z 457.3 [MH+]

### 8-(2-isobutoxybenzyl)-2-[(3-methylisoxazol-4-yl)carbonyl]-2,8-diazaspiro[4.5]decane trifluoroacetate

LC-MS (Method A) RT: 4.27 min, m/z 412.3 [MH+]

### 8-(2-isobutoxybenzyl)-2-(2-methyl-3-furoyl)-2,8-diazaspiro[4.5]decane trifluoroacetate

LC-MS (Method A) RT: 4.24 min, m/z 411.3 [MH+]

### 8-(2-isobutoxybenzyl)-2-[(5-methylisoxazol-4-yl)carbonyl]-2,8-diazaspiro[4.5]decane trifluoroacetate

LC-MS (Method A) RT: 4.02 min, m/z 412.2 [MH+]

### 2-(1,3-benzodioxol-5-ylacetyl)-8-(2-isobutoxybenzyl)-2,8-diazaspiro[4.5]decane trifluoroacetate

LC-MS (Method A) RT: 4.49 min. m/z 465.3 [MH+]

### 2-[(3,5-dimethylisoxazol-4-yl)carbonyl]-8-(2-isobutoxybenzyl)-2,8-diazaspiro[4.5]decane trifluoroacetate

LC-MS (Method A) RT: 4.03 min, m/z 426.4 [MH+]

### 8-(2-isobutoxybenzyl)-2-[(1,2,5-triethyl-1H-pyrrol-3-yl)carbonyl]-2,8-diazaspiro[4.5]decane bis(trifluoroacetate)

LC-MS (Method A) RT: 4.37 min, m/z 438.3 [MH+]

### 8-(2-isobutoxybenzyl)-2-[(5-nitro-1H-pyrazol-3-yl)carbonyl]-2,8-diazaspiro[4.5]decane bis(trifluoroacetate)

LC-MS (Method A) RT: 4.18 min, m/z 442.3 [MH+]

### 2-[(2,5-difluorophenyl)acetyl]-8-(2-isobutoxybenzyl)-2,8-diazaspiro[4.5]decane trifluoroacetate

LC-MS (Method A) RT: 4.64 min, m/z 457.3 [MH+]

### 2-{[4-(benzyloxy)-3-methoxyphenyl]acetyl}-8-(2-isobutoxybenzyl)-2,8-diazaspiro[4.5]decane trifluoroacetate

LC-MS (Method A) RT: 5.15 min, m/z 557.3 [MH+]

### 8-(2-isobutoxybenzyl)-2-{[4-(trifluoromethoxy)phenyl]acetyl}-2,8-diazaspiro[4.5]decane trifluoroacetate

LC-MS (Method A) RT: 5.07 min, m/z 505.3 [MH+]

### 2-(2,5-dimethyl-3-furoyl)-8-(2-isobutoxybenzyl)-2,8-diazaspiro[4.5]decane trifluoroacetate

LC-MS (Method A) RT: 4.46 min, m/z 425.3 [MH+]

### 8-(2-isobutoxybenzyl)-2-[(4-methylphenyl)acetyl]-2,8-diazaspiro[4.5]decane trifluoroacetate

LC-MS (Method A) RT: 4.65 nm in, m/z 435.3 [MH+]

### 8-(2-isobutoxybenzyl)-2-[(4-isopropylphenyl)acetyl]-2,8-diazaspiro[4.5]decane trifluoroacetate

LC-MS (Method A) RT: 5.18 min, m/z 463.4 [MH+]

### 8-(2-isobutoxybenzyl)-2-{[4-(methylsulfonyl)phenyl]acetyl}-2,8-diazaspiro[4.5]decane trifluoroacetate

LC-MS (Method A) RT: 4.10 min, m/z 499.3 [MH+]

### 8-(2-isobutoxybenzyl)-2-(1H-pyrazol-4-ylcarbonyl)-2,8-diazaspiro[4.5]decane bis(trifluoroacetate)

LC-MS Method A) RT: 3.63 min, m/z 397.3 [MH+]

### 8-(2-isobutoxybenzyl)-2-[(4-nitro-1H-pyrazol-3-yl)carbonyl]-2,8-diazaspiro[4.5]decane bis(trifluoroacetate)

LC-MS (Method A) RT: 4.01 min, m/z 442.3 [MH+]

### (2-{[8-(2-isobutoxybenzyl)-2,8-diazaspiro[4.5]dec-2-yl]carbonyl}phenyl)dimethylamine bis(trifluoroacetate)

LC-MS (Method A) RT: 3.71 min, m/z 450.3 [MH+]

### 2-[(3,5-dimethylphenyl)acetyl]-8-(2-isobutoxybenzyl)-2,8-diazaspiro[4.5]decane trifluoroacetate

LC-MS (Method A) RT: 4.95 min, m/z 449.4 [MH+]

### 2-(3-chloro-4-methylbenzoyl)-8-(2-isobutoxybenzyl)-2,8-diazaspiro[4.5]decane trifluoroacetate

LC-MS (Method A) RT: 4.82 min, m/z 455.3 [MH+]

### 8-(2-isobutoxybenzyl)-2-[(4-methoxy-3-thienyl)carbonyl]-2,8-diazaspiro[4.5]decane trifluoroacetate

LC-MS (Method A) RT: 4.24 min, m/z 443.3 [MH+]

### 8-(2-isobutoxybenzyl)-2-{[3-(trifluoromethyl)phenyl]acetyl}-2,8-diazaspiro[4.5]decane trifluoroacetate

LC-MS (Method A) RT: 5.00 min, m/z 489.3 [MH+]

### 8-[(6-chloropyridin-3-yl)carbonyl]-2-(2-isobutoxybenzyl)-2,8-diazaspiro[4.5]decane bis(trifluoroacetate)

LC-MS (Method A) RT: 4.41 min, m/z 442.3 [MH+]

### (4-{[2-(2-isobutoxybenzyl)-2,8-diazaspiro[4.5]dec-8-yl]carbonyl}phenyl)dimethylamine bis(trifluoroacetate)

LC-MS (Method A) RT: 3.97 min, m/z 450.3 [MH+]

### 2-(2-isobutoxybenzyl)-8-[4-(methylsulfonyl)benzoyl]-2,8-diazaspiro[4.5]decane trifluoroacetate

LC-MS (Method A) RT: 4.09 min, m/z 485.3 [MH+]

### 8-(4-butoxybenzoyl)-2-(2-isobutoxybenzyl)-2,8-diazaspiro[4.5]decane trifluoroacetate

LC-MS (Method A) RT: 5.28 min, m/z 479.4 [MH+]

### 1-(4-{[2-(2-isobutoxybenzyl)-2,8-diazaspiro[4.5]dec-8-yl]carbonyl}phenyl)ethanone trifluoroacetate

LC-MS (Method A) RT: 4.30 min, m/z 449.3 [MH+]

### 8-(4-ethylbenzoyl)-2-(2-isobutoxybenzyl)-2,8-diazaspiro[4.5]decane trifluoroacetate

LC-MS (Method A) RT: 4.89 min, m/z 435.3 [MH+]

### 2-{[2-(2-isobutoxybenzyl)-2,8-diazaspiro[4.5]dec-8-yl]carbonyl}quinoline bis(trifluoroacetate)

LC-MS (Method A) RT: 4.47 min, m/z 458.3 [MH+]

### 8-(4-chloro-2-methoxybenzoyl)-2-(2-isobutoxybenzyl)-2,8-diazaspiro[4.5]decane trifluoroacetate

LC-MS (Method A) RT: 4.74 min, m/z 471.3 [MH+]

### 3-{[2-(2-isobutoxybenzyl)-2,8-diazaspiro[4.5]dec-8-yl]carbonyl}benzonitrile trifluoroacetate

LC-MS (Method A) RT: 4.36 min, m/z 432.3 [MH+]

### 2-(2-isobutoxybenzyl)-8-(3-phenoxybenzoyl)-2,8-diazaspiro[4.5]decane trifluoroacetate

LC-MS (Method A) RT: 5.16 min, m/z 499.3 [MH+]

### 8-(4-tert-butylbenzoyl)-2-(2-isobutoxybenzyl)-2,8-diazaspiro[4.5]decane trifluoroacetate

LC-MS (Method A) RT: 5.31 min, m/z 463.4 [MH+]

### 4-{[2-(2-isobutoxybenzyl)-2,8-diazaspiro[4.5]dec-8-yl]carbonyl}benzonitrile trifluoroacetate

LC-MS (Method A) RT: 4.36 min, m/z 432.3 [MH+]

### 2-(2-isobutoxybenzyl)-8-(4-morpholin-4-ylbenzoyl)-2,8-diazaspiro[4.5]decane bis(trifluoroacetate)

LC-MS (Method A) RT: 4.21 min, m/z 492.3 [MH+]

### 2-(2-isobutoxybenzyl)-8-(6-methoxy-2-naphthoyl)-2,8-diazaspiro[4,5]decane trifluoroacetate

LC-MS (Method A) RT: 4.94 min, m/z 487.3 [MH+]

### 8-(2,3-dichlorobenzoyl)-2-(2-isobutoxybenzyl)-2,8-diazaspiro[4.5]decane trifluoroacetate

LC-MS (Method A) RT: 4.86 min, m/z 475.2 [MH+]

### 2-(2-isobutoxybenzyl)-8-(3-methoxybenzoyl)-2,8-diazaspiro[4.5]decane trifluoroacetate

LC-MS (Method A) RT: 4.45 min, m/z 437.3 [MH+]

### 8-(2,3-dimethylbenzoyl)-2-(2-isobutoxybenzyl)-2,8-diazaspiro[4.5]decane trifluoroacetate

LC-MS (Method A) RT: 4.74 min, m/z 435.3 [MH+]

### 2-(2-isobutoxybenzyl)-8-(4-methylbenzoyl)-2,8-diazaspiro[4.5]decane trifluoroacetate

LC-MS (Method A) RT: 4.60 min, m/z 421.3 [MH+]

### 8-(3,4-dichlorobenzoyl)-2-(2-isobutoxybenzyl)-2,8-diazaspiro[4.5]decane trifluoroacetate

LC-MS (Method A) RT: 5.02 min, m/z 475.2 [MH+]

### 8-(3,4-dimethoxybenzoyl)-2-(2-isobutoxybenzyl)-2,8-diazaspiro[4.5]decane trifluoroacetate

LC-MS (Method A) RT: 4.30 min, m/z 467.3 [MH+]

### 8-(2,4-dichlorobenzoyl)-2-(2-isobutoxybenzyl)-2,8-diazaspiro[4.5]decane trifluoroacetate

LC-MS (Method A) RT: 4.94 min, m/z 475.2 [MH+]

### 2-(2-isobutoxybenzyl)-8-(4-isopropoxybenzoyl)-2,8-diazaspiro[4.5]decane trifluoroacetate

LC-MS (Method A) RT: 4.90 min, m/z 465.3 [MH+]

### 2-(2-isobutoxybenzyl)-8-(2-naphthoyl)-2,8-diazaspiro[4.5]decane trifluoroacetate

LC-MS (Method A) RT: 4.88 min, m/z 457.3 [MH+]

### 8-(2-chlorobenzoyl)-2-(2-isobutoxybenzyl)-2,8-diazaspiro[4.5]decane trifluoroacetate

LC-MS (Method A) RT: 4.64 min, m/z 441.3 [MH+]

### 8-(2,3-dimethoxybenzoyl)-2-(2-isobutoxybenzyl)-2,8-diazaspiro[4.5]decane trifluoroacetate

LC-MS (Method A) RT: 4.42 min, m/z 467.3 [MH+]

### 2-(2-isobutoxybenzyl)-8-(1-naphthoyl)-2,8-diazaspiro[4.5]decane trifluoroacetate

LC-MS (Method A) RT: 4.78 min, m/z 457.3 [MH+]

### (3-{[2-(2-isobutoxybenzyl)-2,8-diazaspiro[4.5]dec-8-yl]carbonyl}phenyl)dimethylamine bis(trifluoroacetate)

LC-MS (Method A) RT: 3.77 min, m/z 450.3 [MH+]

### 2-(2-isobutoxybenzyl)-8-(4-methoxybenzoyl)-2,8-diazaspiro[4.5]decane trifluoroacetate

LC-MS (Method A) RT: 4.44 min, m/z 437.3 [MH+]

### N,N-diethyl-4-{[2-(2-isobutoxybenzyl)-2,8-diazaspiro[4.5]dec-8-yl]carbonyl}aniline bis(trifluoroacetate)

LC-MS (Method A) RT: 3.74 min, m/z 478.4 [MH+]

### 2-(2-isobutoxybenzyl)-8-(4-propylbenzoyl)-2,8-diazaspiro[4.5]decane trifluoroacetate

LC-MS (Method A) RT: 5.18 min, m/z 449.4 [MH+]

### 8-(2-chloroisonicotinoyl)-2-(2-isobutoxybenzyl)-2,8-diazaspiro[4.5]decane bis(trifluoroacetate)

LC-MS (Method A) RT: 4.21 min, m/z 442.3 [MH+]

### 2-(2-isobutoxybenzyl)-8-[3-(trifluoromethyl)benzoyl]-2,8-diazaspiro[4.5]decane trifluoroacetate

LC-MS (Method A) RT: 4.94 min, m/z 475.3 [MH+]

### 2-(2-isobutoxybenzyl)-8-[4-(trifluoromethyl)benzoyl]-2,8-diazaspiro[4.5]decane trifluoroacetate

LC-MS (Method A) RT: 4.94 min, m/z 475.3 [MH+]

### 4-{[2-(2-isobutoxybenzyl)-2,8-diazaspiro[4.5]dec-8-yl]carbonyl}quinoline bis(trifluoroacetate)

LC-MS (Method A) RT: 3.74 min, m/z 458.4 [MH+]

### 8-(3-chloro-2-methylbenzoyl)-2-(2-isobutoxybenzyl)-2,8-diazaspiro[4.5]decane trifluoroacetate

LC-MS (Method A) RT: 4.82 min, m/z 455.3 [MH+]

### (4-{2-[2-(2-isobutoxybenzyl)-2,8-diazaspiro[4.5]dec-8-yl]-2-oxoethyl}phenyl)dimethylamine bis(trifluoroacetate)

LC-MS (Method A) RT: 3.56 min, m/z 464.4 [MH+]

### 8-[(2-fluorophenyl)acetyl]-2-(2-isobutoxybenzyl)-2,8-diazaspiro[4.5]decane trifluoroacetate

LC-MS (Method A) RT: 4.59 min, m/z 439.3 [MH+]

### 2-(2-isobutoxybenzyl)-8-[(3-nitrophenyl)acetyl]-2,8-diazaspiro[4.5]decane trifluoroacetate

LC-MS (Method A) RT: 4.60 min, m/z 466.3 [MH+]

### 2-(2-isobutoxybenzyl)-8-[(4-nitrophenyl)acetyl]-2,8-diazaspiro[4.5]decane trifluoroacetate

LC-MS (Method A) RT: 4.61 min, m/z 466.3 [MH+]

### 2-(2-isobutoxybenzyl)-8-[(2-nitrophenyl)acetyl]-2,8-diazaspiro[4.5]decane trifluoroacetate

LC-MS (Method A) RT: 4.62 min, m/z 466.4 [MH+]

### 8-[(3,4-dimethoxyphenyl)acetyl]-2-(2-isobutoxybenzyl)-2,8-diazaspiro[4.5]decane trifluoroacetate

LC-MS (Method A) RT: 4.30 min, m/z 481.3 [MH+]

### 8-(3-furoyl)-2-(2-isobutoxybenzyl)-2,8-diazaspiro[4.5]decane trifluoroacetate

LC-MS (Method A) RT: 4.14 min, m/z 397.3 [MH+]

### 8-[(4-chlorophenyl)acetyl]-2-(2-isobutoxybenzyl)-2,8-diazaspiro[4.5]decane trifluoroacetate

LC-MS (Method A) RT: 4.82 min, m/z 455.3 [MH+]

### 2-(2-isobutoxybenzyl)-8-(1,2,3-thiadiazol-4-ylcarbonyl)-2,8-diazaspiro[4.5]decane bis(trifluoroacetate)

LC-MS (Method A) RT: 3.99 min, m/z 415.3 [MH+]

### 2-(2-isobutoxybenzyl)-8-[(5-methyl-1H-pyrazol-3-yl)carbonyl]-2,8-diazaspiro[4.5]decane bis(trifluoroacetate)

LC-MS (Method A) RT: 3.84 min, m/z 411.3 [MH+]

### 8-[(1,5-dimethyl-1H-pyrazol-3-yl)carbonyl]-2-(2-isobutoxybenzyl)-2,8-diazaspiro[4.5]decane bis(trifluoroacetate)

LC-MS (Method A) RT: 4.03 min, m/z 425.3 [MH+]

### 8-[(4-butoxyphenyl)acetyl]-2-(2-isobutoxybenzyl)-2,8-diazaspiro[4.5]decane trifluoroacetate

LC-MS (Method A) RT: 5.32 min, m/z 493.4 [MH+]

### 8-[(3,5-difluorophenyl)acetyl]-2-(2-isobutoxybenzyl)-2,8-diazaspiro[4.5]decane trifluoroacetate

LC-MS (Method A) RT: 4.73 min, m/z 457.3 [MH+]

### 8-[(2,4-dichlorophenyl)acetyl]-2-(2-isobutoxybenzyl)-2,8-diazaspiro[4.5]decane trifluoroacetate

LC-MS (Method A) RT: 5.07 min, m/z 489.2 [MH+]

### 8-[(2,4-difluorophenyl)acetyl]-2-(2-isobutoxybenzyl)-2,8-diazaspiro[4.5]decane trifluoroacetate

LC-MS (Method A) RT: 4.70 min, m/z 457.3 [MH+]

### 2-(2-isobutoxybenzyl)-8-[(3-methylisoxazol-4-yl)carbonyl]-2,8-diazaspiro[4.5]decane trifluoroacetate

LC-MS (Method A) RT: 4.23 min, m/z 412.3 [MH+]

### 2-(2-isobutoxybenzyl)-8-(2-methyl-3-furoyl)-2,8-diazaspiro[4.5]decane trifluoroacetate

LC-MS (Method A) RT: 4.30 min, m/z 411.3 [MH+]

### 2-(2-isobutoxybenzyl)-8-[(5-methylisoxazol-4-yl)carbonyl]-2,8-diazaspiro[4.5]decane trifluoroacetate

LC-MS (Method A) RT: 4.57 min, m/z 412.3 [MH+]

### 8-(1,3-benzodioxol-5-ylacetyl)-2-(2-isobutoxybenzyl)-2,8-diazaspiro[4.5]decane trifluoroacetate

LC-MS (Method A) RT: 4.47 min, m/z 465.3 [MH+]

### 2-(2-isobutoxybenzyl)-8-[(1,2,5-trimethyl-1H-pyrrol-3-yl)carbonyl]-2,8-diazaspiro[4.5]decane bis(trifluoroacetate)

LC-MS (Method A) RT: 4.45 min, m/z 438.3 [MH+]

### 2-(2-isobutoxybenzyl)-8-[(5-nitro-1H-pyrazol-3-yl)carbonyl]-2,8-diazaspiro[4.5]decane bis(trifluoroacetate)

LC-MS (Method A) RT: 4.20 min, m/z 442.3 [MH+]

### 8-[(2,5-difluorophenyl)acetyl]-2-(2-isobutoxybenzyl)-2,8-diazaspiro[4.5]decane trifluoroacetate

LC-MS (Method A) RT: 4.69 min, m/z 457.3 [MH+]

### 8-{[4-(benzyloxy)-3-methoxyphenyl]acetyl}-2-(2-isobutoxybenzyl)-2,8-diazaspiro[4.5]decane trifluoroacetate

LC-MS (Method A) RT: 5.11 min. m/z 557.4 [MH+]

### 2-(2-isobutoxybenzyl)-8-{[4-(trifluoromethoxy)phenyl]acetyl}-2,8-diazaspiro[4.5]decane trifluoroacetate

LC-MS (Method A) RT: 5.12 min, m/z 505.3 [MH+]

### 8-(2,5-dimethyl-3-furoyl)-2-(2-isobutoxybenzyl)-2,8-diazaspiro[4.5]decane trifluoroacetate

LC-MS (Method A) RT: 4.53 min, m/z 425.3 [MH+]

### 2-(2-isobutoxybenzyl)-8-[(4-methylphenyl)acetyl]-2,8-diazaspiro[4.5]decane trifluoroacetate

LC-MS (Method A) RT: 4.75 min, m/z 435.3 [MH+]

### 2-(2-isobutoxybenzyl)-8-(3-thienylcarbonyl)-2,8-diazaspiro[4.5]decane trifluoroacetate

LC-MS (Method A) RT: 4.29 min, m/z 413.3 [MH+]

### 2-(2-isobutoxybenzyl)-8-(pyridin-4-ylacetyl)-2,8-diazaspiro[4.5]decane bis(trifluoroacetate)

LC-MS (Method A) RT: 3.40 min, m/z 422.3 [MH+]

### 2-(2-isobutoxybenzyl)-8-(pyridin-2-ylacetyl)-2,8-diazaspiro[4.5]decane bis(trifluoroacetate)

LC-MS (Method A) RT: 0.07 min, m/z 422.3 [MH+]

### 2-(2-isobutoxybenzyl)-8-[(4-isopropylphenyl)acetyl]-2,8-diazaspiro[4.5]decane trifluoroacetate

LC-MS (Method A) RT: 5.18 min, m/z 463.4 [MH+]

### 2-(2-isobutoxybenzyl)-8-{[4-(methylsulfonyl)phenyl]acetyl}-2,8-diazaspiro[4.5]decane trifluoroacetate

LC-MS (Method A) RT: 4.14 min, m/z 499.3 [MH+]

### 2-(2-isobutoxybenzyl)-8-(1H-pyrazol-4-ylcarbonyl)-2,8-diazaspiro[4.5]decane bis(trifluoroacetate)

LC-MS (Method A) RT: 3.69 min, m/z 397.3 [MH+]

### 2-(2-isobutoxybenzyl)-8-[(4-nitro-1H-pyrazol-3-yl)carbonyl]-2,8-diazaspiro[4.5]decane bis(trifluoroacetate)

LC-MS (Method A) RT: 4.03 min, m/z 442.3 [MH+]

### (2-{[2-(2-isobutoxybenzyl)-2,8-diazaspiro[4.5]dec-8-yl]carbonyl}phenyl)dimethylamine bis(trifluoroacetate)

LC-MS (Method A) RT: 3.75 min, m/z 450.3 [MH+]

Pharmacological Data

CCL1 SPA Binding Assay

Membranes from CHO-K1 cells transfected with human recombinant chemokine CCR8 receptor (ES-136-M) were purchased from Euroscreen. Membrane preparations are stored at −70C in 7.5 mM Tris-Cl pH 7.5, 12.5 mM MgCl₂, 0.3 mM EDTA, 1 mM EGTA, 250 mM sucrose until used.

The CCR8 membranes (50.6 mg/ml) were preincubated with Wheat Germ Agglutinin SPA beads (4.05 mg/ml) in assay buffer (50 mM HEPES, 1 mM CaCl₂x2H₂O, 5 mM MgCl₂x6H₂O, 75 mM NaCl, 0.1% BSA) at pH=7.4 for 2 hours on ice. A 10-point dose-response curve (final concentrations 50 μM, 16.7 μM, 5.6 μM, 1.9 μM, 0.62 μM, 0.21 μM, 0.069 μM, 0.023 μM) was prepared by diluting compounds by serial dilution 1:3 in DMSO. In the screening plate (Polystyrene NBS plates, Costar Corning 3604) 1 μl from the DMSO solutions of compounds was transferred into each well. 1 μl of DMSO was added to the blank control wells and 1 μl unlabeled CCL1 (300 nM) was added to background control wells. 50 μl of the SPA bead-membrane mixture was added into each well. Finally, 50 μl (30 pM) ¹²⁵I CCL1 (2000 Ci/mM) was added to each well. Plates were then incubated at RT with shaking (700 rpm) for 90 minutes followed by 30 minutes at RT without shaking. The plate was read in a Wallac MicroBeta counter for 2 minutes/well.

Typical Data

| Compound | IC₅₀ (nM) |
|---|---|
| 3-(4-chlorobenzoyl)-9-(2-phenoxybenzyl)- | 81 |
| 3,9-diazaspiro[5.5]undecane | |
| trifluoroacetate | |
| 3-Benzoyl-9-(2-propoxybenzyl)-3,9- | 165 |
| diazaspiro[5.5]undecane trifluoroacetate | |
| 3-benzoyl-9-{2-[(3,5-dimethylisoxazol-4- | 710 |
| yl)methoxy]benzyl}-3,9- | |
| diazaspiro[5.5]undecane trifluoroacetate | |

All the compounds of the examples have an IC₅₀ of less than 40 μM.

1. A compound of formula (I) and pharmaceutically acceptable salts, solvates or N-oxides thereof: 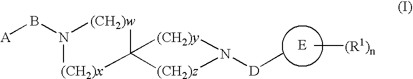
in which:
w, x, y and z are independently 1, 2 or 3;
A is a phenyl, benzyl, alkyl, C₃₋₆ saturated or partially unsaturated cycloalkyl, a 6-membered-cycloheteroalkyl ring containing 1 or 2 heteroatoms selected from O or N, alkyl-aryl, naphthyl, a 5- to 7-membered heteroaromatic ring containing 1 to 3 heteroatoms, a 9- or 10-membered bicyclic heteroaromatic ring containing 1 to 4 heteroatoms, a phenyl-fused-5 to 6-membered cycloheteroalkyl containing at least one heteroatom selected from O, S or N, or pyridone;
A being optionally substituted by one or more groups selected from
halogen, cyano, CF₃, OCF₃, C₁₋₆ alkoxy, hydroxy, C₁₋₆ alkyl, C₁₋₆ thioalkyl, SO₂C₁₋₆ alkyl, NR²R³, amide, C₁₋₆ alkoxycarbonyl, —NO₂, C₁₋₆ acylamino, —CO₂H, C₁₋₆ carboxyalkyl, morpholine;
phenoxy optionally substituted with one or more groups selected from halogen, C₁₋₆ alkoxy, C₁₋₆ alkyl;
phenyl or diphenyl, said phenyl and diphenyl independently being optionally substituted with one or more groups independently selected from halogen, C₁₋₆ alkoxy, C₁₋₆ alkyl, or —COOH;
benzyloxy optionally substituted with one or more groups selected from halogen, C₁₋₆ alkoxy, C₁₋₆ alkyl;
or a 5 to 7 membered heteroaromatic ring containing 1 to 4 heteroatoms selected from O, S or N optionally substituted with one or more groups independently selected from halogen, C₁₋₆ alkoxy, C₁₋₆ alkyl;
R² and R³ are independently halogen or C₁₋₆ alkyl, or R² and R³ together with the nitrogen to which they are attached form a 6-membered saturated ring optionally containing a further heteroatom;
B is a group R⁴-R⁵ where
R⁴ is a bond, —N(R⁶)—, —R⁷—N(R⁸)—, —N(R⁹)—R¹⁰—, O, C₁₋₄ alkyl optionally interrupted by N(R¹¹) or O, C₂₋₄ alkenyl or 1,3-butadienyl, or —SO₂—N(R¹²)—;
R⁵ is C═O or SO₂;
R⁶, R⁸, R¹¹, and R¹² are each independently H or C₁₋₆ alkyl;
R⁹ is H, C₁₋₆ alkyl or C₁₋₆ carboxyalkyl;
R⁷ and R¹⁰ are independently C₁₋₄ alkyl or C₃₋₅ cycloalkyl;
D is C₁₋₄ alkyl;
E is phenyl, or a 5- or 6-membered aromatic ring containing one or two heteroatoms;
Each R¹ independently represents C₁₋₆ alkoxy optionally substituted with one or more halogens, C₄₋₆cycloalkylalkoxy, C₂₋₆ alkenyloxy, halogen, OCH₂CN, COC₁₋₆ alkyl, OR¹¹, OCH₂R¹¹, or —S—R¹²;
R¹¹ is a phenyl or 5- or 6-membered saturated or aromatic ring containing one or two heteroatoms and each optionally substituted by one or more groups selected from C₁₋₆ alkyl, halogen, C₁₋₆ alkoxy, CF₃, or cyano;
R¹² is C₁₋₆ alkyl or R¹² is phenyl optionally substituted with one or more halogens, and
n is 0, 1, 2, 3 or 4;
provided that when E is phenyl, w+x is greater than 2 and n is 1 then R¹ is not a phenoxy group at the meta-position of the phenyl ring E,
and provided that when A-B is acetyl, tosyl or tertiary butyloxy-carbonyl (t-boc), then D-E-(R¹)ₙ is not benzyl.

2. A compound of formula (I′) and pharmaceutically acceptable salts, solvates or N-oxides thereof: 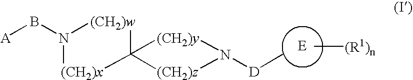
in which:
w, x, y and z are independently 1, 2 or 3;
A is a phenyl, benzyl, alkyl, C₃₋₆ saturated or partially unsaturated cycloalkyl, a 6-membered-cycloheteroalkyl ring containing 1 or 2 heteroatoms selected from O or N, alkyl-aryl, naphthyl, a 5- to 7-membered heteroaromatic ring containing 1 to 3 heteroatoms, a 9- or 10-membered bicyclic heteroaromatic ring containing 1 to 4 heteroatoms, a phenyl-fused-5 to 6-membered cycloheteroalkyl containing at least one heteroatom selected from O, S or N, or pyridone;
A being optionally substituted by one or more groups selected from
halogen, cyano, CF₃, OCF₃, C₁₋₆ alkoxy, hydroxy, C₁₋₆ alkyl, C₁₋₆ thioalkyl, SO₂C₁₋₆ alkyl, NR²R³, amide, C₁₋₆ alkoxycarbonyl, —NO₂, C₁₋₆ acylamino, —CO₂H, C₁₋₆ carboxyalkyl, morpholine;
phenoxy optionally substituted with one or more groups selected from halogen, C₁₋₆ alkoxy, C₁₋₆ alkyl;
phenyl or diphenyl, said phenyl and diphenyl independently being optionally substituted with one or more groups independently selected from halogen, C₁₋₆ alkoxy, C₁₋₆ alkyl, or —COOH;
benzyloxy optionally substituted with one or more groups selected from halogen, C₁₋₆ alkoxy, C₁₋₆ alkyl;
or a 5 to 7 membered heteroaromatic ring containing 1 to 4 heteroatoms selected from O, S or N optionally substituted with one or more groups independently selected from halogen, C₁₋₆ alkoxy, C₁₋₆ alkyl;
R² and R³ are independently halogen or C₁₋₆ alkyl, or R² and R³ together with the nitrogen to which they are attached form a 6-membered saturated ring optionally containing a further heteroatom;
B is a group R⁴-R⁵ where
R⁴ is a bond, —N(R⁶)—, —R⁷—N(R⁸)—, —N(R⁹)—R¹⁰—, O, C₁₋₄ alkyl optionally interrupted by N(R¹¹) or O, C₂₋₄ alkenyl or 1,3-butadienyl, or —SO₂—N(R¹²)—;
R⁵ is C═O or SO₂;
R⁶, R⁸, R¹¹, and R¹² are each independently H or C₁₋₆ alkyl;
R⁹ is H, C₁₋₆ alkyl or C₁₋₆ carboxyalkyl;
R⁷ and R¹⁰ are independently C₁₋₄ alkyl or C₃₋₅ cycloalkyl;
D is C₁₋₄ alkyl;
E is phenyl, or a 5- or 6-membered aromatic ring containing one or two heteroatoms;
Each R¹ independently represents C₁₋₆ alkoxy optionally substituted with one or more halogens, C₄₋₆ cycloalkylalkoxy, C₂₋₆ alkenyloxy, halogen, OCH₂CN, COC₁₋₆ alkyl, OR¹¹, OCH₂R¹¹, or —S—R¹²;
R¹¹ is a phenyl or 5- or 6-membered saturated or aromatic ring containing one or two heteroatoms and each optionally substituted by one or more groups selected from C₁₋₆ alkyl, halogen, C₁₋₆ alkoxy, CF₃, or cyano;
R¹² is C₁₋₆ alkyl or R¹² is phenyl optionally substituted with one or more halogens, and
n is 0, 1, 2, 3 or 4;
provided that when E is phenyl and n is 1 then R¹ is not a phenoxy group at the meta-position of the phenyl ring E,
and provided that when A-B is acetyl, tosyl or tertiary butyloxy-carbonyl (t-boc), then D-E-(R¹)ₙ is not benzyl.

3. A compound according to claim 1, wherein w+x is not greater than 4 and y+z is not greater than 4

4. A compound according to claim 1, wherein A is phenyl, pyridyl, or pyrimidyl, each being optionally substituted by one or more groups selected from:
halogen, cyano, CF₃, OCF₃, C₁₋₆ alkoxy, hydroxy, C₁₋₆ alkyl, C₁₋₆ thioalkyl, SO₂C₁₋₆ alkyl, NR²R³, amide, C₁₋₆ alkoxycarbonyl, —NO₂, C₁₋₆ acylamino, —CO₂H, C₁₋₆ carboxyalkyl, morpholine;
phenoxy optionally substituted with one or more groups selected from halogen, C₁₋₆ alkoxy, C₁₋₆ alkyl;
phenyl or diphenyl, said phenyl and diphenyl independently being optionally substituted with one or more groups independently selected from halogen, C₁₋₆ alkoxy, C₁₋₆ alkyl, or —COOH;
benzyloxy optionally substituted with one or more groups selected from halogen, C₁₋₆ alkoxy, C₁₋₆ alkyl;
or a 5 to 7 membered heteroaromatic ring containing 1 to 4 heteroatoms selected from O, S or N optionally substituted with one or more groups independently selected from halogen, C₁₋₆ alkoxy, C₁₋₆ alkyl.

5. A compound according to claim 1, wherein R¹ is OCH₂CH═CH₂, butyloxy, propyloxy, cyclopropylmethoxy, benzyloxy, ethoxy, bromo, methyl, chloro, OCH₂CN, fluoro, methoxy, CF₃; or
OCH₂R¹¹ where R¹¹ is tetrahydrofuran, tetrahydropyran, chlorothiazole or dimethyloxazole.

6. A compound according to claim 1, wherein when E is phenyl or a 6-membered aromatic ring containing 1 or 2 heteroatoms, and R¹ is phenoxy, the phenoxy is present in the ortho position of ring E.

7. A compound selected from the group consisting of: 3-(2-ethoxybenzyl)-9-[3-(trifluoromethyl)benzoyl]-3,9-diazaspiro[5.5]undecane,
3-benzoyl-9-(2-ethoxybenzyl)-3,9-diazaspiro[5.5]undecane,
3-(2-ethoxybenzyl)-9-(4-ethylbenzoyl)-3,9-diazaspiro[5.5]undecane,
3-[(6-chloropyridin-3-yl)carbonyl]-9-(2-ethoxybenzyl)-3,9-diazaspiro[5.5]undecane,
(4-{[9-(2-ethoxybenzyl)-3,9-diazaspiro[5.5]undec-3-yl]carbonyl}phenyl)dimethylamine,
3-(2-ethoxybenzyl)-9-[2-methoxy-4-(methylthio)benzoyl]-3,9-diazaspiro[5.5]undecane,
3-(4-butoxybenzoyl)-9-(2-ethoxybenzyl)-3,9-diazaspiro[5.5]undecane,
1-(4-{[9-(2-ethoxybenzyl)-3,9-diazaspiro[5.5]undec-3-yl]carbonyl}phenyl)ethanone,
3-(2-ethoxybenzyl)-9-(quinolin-2-ylcarbonyl)-3,9-diazaspiro[5.5]undecane,
3-(2-ethoxybenzyl)-9-(3-phenoxybenzoyl)-3,9-diazaspiro[5.5]undecane,
3-(4-tert-butylbenzoyl)-9-(2-ethoxybenzyl)-3,9-diazaspiro[5.5]undecane,
4-{[9-(2-ethoxybenzyl)-3,9-diazaspiro[5.5]undec-3-yl]carbonyl}benzonitrile,
3-(2-ethoxybenzyl)-9-(6-methoxy-2-naphthoyl)-3,9-diazaspiro[5.5]undecane,
3-(2,3-dichlorobenzoyl)-9-(2-ethoxybenzyl)-3,9-diazaspiro[5.5]undecane,
3-(2-ethoxybenzyl)-9-(3-methoxybenzoyl)-3,9-diazaspiro[5.5]undecane,
3-(2,3-dimethylbenzoyl)-9-(2-ethoxybenzyl)-3,9-diazaspiro[5.5]undecane,
3-(4-chlorobenzoyl)-9-(2-ethoxybenzyl)-3,9-diazaspiro[5.5]undecane,
3-(2-ethoxybenzyl)-9-(4-methylbenzoyl)-3,9-diazaspiro[5.5]undecane,
3-(3,4-dichlorobenzoyl)-9-(2-ethoxybenzyl)-3,9-diazaspiro[5.5]undecane,
3-(3,4-dimethoxybenzoyl)-9-(2-ethoxybenzyl)-3,9-diazaspiro[5.5]undecane,
3-(2,4-dichlorobenzoyl)-9-(2-ethoxybenzyl)-3,9-diazaspiro[5.5]undecane,
3-(2-ethoxybenzyl)-9-(4-isopropoxybenzoyl)-3,9-diazaspiro[5.5]undecane,
3-(2-ethoxybenzyl)-9-(4-phenoxybenzoyl)-3,9-diazaspiro[5.5]undecane,
3-(2-ethoxybenzyl)-9-(2-naphthoyl)-3,9-diazaspiro[5.5]undecane,
3-(2-chlorobenzoyl)-9-(2-ethoxybenzyl)-3,9-diazaspiro[5.5]undecane,
3-(2,3-dimethoxybenzoyl)-9-(2-ethoxybenzyl)-3,9-diazaspiro[5.5]undecane,
3-(2-ethoxybenzyl)-9-(1-naphthoyl)-3,9-diazaspiro[5.5]undecane,
(3-{[9-(2-ethoxybenzyl)-3,9-diazaspiro[5.5]undec-3-yl]carbonyl}phenyl)dimethylamine,
3-(2-ethoxybenzyl)-9-[3-(methylsulfonyl)benzoyl]-3,9-diazaspiro[5.5]undecane,
3-(2-ethoxybenzyl)-9-(4-methoxybenzoyl)-3,9-diazaspiro[5.5]undecane,
(4-{[9-(2-ethoxybenzyl)-3,9-diazaspiro[5.5]undec-3-yl]carbonyl}phenyl)diethylamine,
3-(2-ethoxybenzyl)-9-(4-propylbenzoyl)-3,9-diazaspiro[5.5]undecane,
3-(2-chloroisonicotinoyl)-9-(2-ethoxybenzyl)-3,9-diazaspiro[5.5]undecane,
3-(2-ethoxybenzyl)-9-[3-(trifluoromethyl)benzoyl]-3,9-diazaspiro[5.5]undecane,
and pharmaceutically acceptable salts and solvates thereof.

8. A pharmaceutical composition comprising a compound as claimed in claim 1 or a pharmaceutically acceptable salt or solvate thereof, in association with a pharmaceutically acceptable adjuvant, diluent or carrier.

9. A process for the preparation of a pharmaceutical composition comprising a compound as claimed in claim 1 or a pharmaceutically acceptable salt or solvate thereof, in association with a pharmaceutically acceptable adjuvant, diluent or carrier which comprises mixing a compound as claimed claim 1 or a pharmaceutically acceptable salt or solvate thereof, with a pharmaceutically acceptable adjuvant, diluent or carrier.

10-11. (canceled)

12. A method of treating a chemokine mediated disease wherein the chemokine binds to one or more chemokine receptors, which comprises administering to a patient a therapeutically effective amount of a compound as claimed in claim 1, or a pharmaceutically acceptable salt or solvate thereof.

13. A method according to claim 12 in which the chemokine receptor belongs to the CCR chemokine receptor subfamily.

14. A method according to claim 12 in which the chemokine receptor is the CCR8 receptor.

15. A method according to claim 14 wherein the disease is asthma.

16. (canceled)

17. A process for the preparation of a compound as defined in claim 1, and optical isomers, racemates and tautomers thereof and pharmaceutically acceptable salts thereof, which comprises:
(a) reaction of a compound of formula (II): 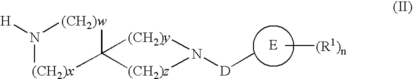
where R¹, D and E are as defined in formulae (I) or (I′) or are protected derivatives thereof, with a compound of formula (III):
A-B-LG (III)
where A and B are as defined in formulae (I) or (I′) or are protected derivatives thereof and LG is a leaving group.

18. A compound selected from the group consisting of:
3-(2-ethoxybenzyl)-9-[4-(trifluoromethyl)benzoyl]-3,9-diazaspiro[5.5]undecane,
3-(2-ethoxybenzyl)-9-(quinolin-4-ylcarbonyl)-3,9-diazaspiro[5.5]undecane,
3-(3-chloro-2-methylbenzoyl)-9-(2-ethoxybenzyl)-3,9-diazaspiro[5.5]undecane,
3-[2-(benzyloxy)benzyl]-9-[(6-chloropyridin-3-yl)carbonyl]-3,9-diazaspiro[5.5]undecane,
[4-({9-[2-(benzyloxy)benzyl]-3,9-diazaspiro[5.5]undec-3-yl}carbonyl)phenyl]dimethylamine,
3-[2-(benzyloxy)benzyl]-9-[2-methoxy-4-(methylthio)benzoyl]-3,9-diazaspiro[5.5]undecane,
1-[4-({9-[2-(benzyloxy)benzyl]-3,9-diazaspiro[5.5]undec-3-yl}carbonyl)phenyl]ethanone.
3-[2-(benzyloxy)benzyl]-9-(4-ethylbenzoyl)-3,9-diazaspiro[5.5]undecane,
3-[2-(benzyloxy)benzyl]-9-(quinolin-2-ylcarbonyl)-3,9-diazaspiro[5.5]undecane,
3-[2-(benzyloxy)benzyl]-9-(4-chloro-2-methoxybenzoyl)-3,9-diazaspiro[5.5]undecane,
3-({9-[2-(benzyloxy)benzyl]-3,9-diazaspiro[5.5]undec-3-yl}carbonyl)benzonitrile,
3-[2-(benzyloxy)benzyl]-9-(4-tert-butylbenzoyl)-3,9-diazaspiro[5.5]undecane,
4-({9-[2-(benzyloxy)benzyl]-3,9-diazaspiro[5.5]undec-3-yl}carbonyl)benzonitrile,
3-[2-(benzyloxy)benzyl]-9-(4-morpholin-4-ylbenzoyl)-3,9-diazaspiro[5.5]undecane,
3-[2-(benzyloxy)benzyl]-9-(2,3-dichlorobenzoyl)-3,9-diazaspiro[5.5]undecane,
3-[2-(benzyloxy)benzyl]-9-(3-methoxybenzoyl)-3,9-diazaspiro[5.5]undecane,
3-[2-(benzyloxy)benzyl]-9-(2,3-dimethylbenzoyl)-3,9-diazaspiro[5.5]undecane,
3-[2-(benzyloxy)benzyl]-9-(4-chlorobenzoyl)-3,9-diazaspiro[5.5]undecane,
3-[2-(benzyloxy)benzyl]-9-(4-methylbenzoyl)-3,9-diazaspiro[5.5]undecane,
3-[2-(benzyloxy)benzyl]-9-(3,4-dimethoxybenzoyl)-3,9-diazaspiro[5.5]undecane,
3-[2-(benzyloxy)benzyl]-9-(4-isopropoxybenzoyl)-3,9-diazaspiro[5.5]undecane,
3-[2-(benzyloxy)benzyl]-9-(4-phenoxybenzoyl)-3,9-diazaspiro[5.5]undecane,
3-[2-(benzyloxy)benzyl]-9-(2-naphthoyl)-3,9-diazaspiro[5.5]undecane,
3-[2-(benzyloxy)benzyl]-9-(2-chlorobenzoyl)-3,9-diazaspiro[5.5]undecane,
3-[2-(benzyloxy)benzyl]-9-(2,3-dimethoxybenzoyl)-3,9-diazaspiro[5.5]undecane,
3-[2-(benzyloxy)benzyl]-9-(1-naphthoyl)-3,9-diazaspiro[5.5]undecane,
[3-({9-[2-(benzyloxy)benzyl]-3,9-diazaspiro[5.5]undec-3-yl}carbonyl)phenyl]dimethylamine,
3-[2-(benzyloxy)benzyl]-9-(4-methoxybenzoyl)-3,9-diazaspiro[5.5]undecane,
[4-({9-[2-(benzyloxy)benzyl]-3,9-diazaspiro[5.5]undec-3-yl}carbonyl)phenyl]-diethylamine,
3-[2-(benzyloxy)benzyl]-9-(2-chloroisonicotinoyl)-3,9-diazaspiro[5.5]undecane,
3-[2-(benzyloxy)benzyl]-9-[3-(trifluoromethyl)benzoyl]-3,9-diazaspiro[5.5]undecane,
3-[2-(benzyloxy)benzyl]-9-[4-(trifluoromethyl)benzoyl]-3,9-diazaspiro[5.5]undecane,
3-[2-(benzyloxy)benzyl]-9-(quinolin-4-ylcarbonyl)-3,9-diazaspiro[5.5]undecane,
3-benzoyl-9-(2-propoxybenzyl)-3,9-diazaspiro[5.5]undecane,
3-benzoyl-9-[2-(tetrahydrofuran-2-ylmethoxy)benzyl]-3,9-diazaspiro[5.5]undecane,
3-(2-propoxybenzyl)-9-(pyridin-3-ylcarbonyl)-3,9-diazaspiro[5.5]undecane,
and pharmaceutically acceptable salts and solvates thereof.

19. A compound selected from the group consisting of:
3-(2-isobutoxybenzyl)-9-(pyridin-4-ylcarbonyl)-3,9-diazaspiro[5.5]undecane,
3-(4-chlorobenzoyl)-9-[2-(pyridin-2-ylmethoxy)benzyl]-3,9-diazaspiro[5.5]undecane,
3-{[9-(2-isobutoxybenzyl)-3,9-diazaspiro[5.5]undec-3-yl]carbonyl}benzonitrile,
3-(2-isobutoxybenzyl)-9-(pyrazin-2-ylcarbonyl)-3,9-diazaspiro[5.5]undecane,
3-(2-isobutoxybenzyl)-9-(pyrimidin-2-ylcarbonyl)-3,9-diazaspiro[5.5]undecane,
3-(2-isobutoxybenzyl)-9-(pyrimidin-4-ylcarbonyl)-3,9-diazaspiro[5.5]undecane,
3-(2-isobutoxybenzyl)-9-(pyrimidin-5-ylcarbonyl)-3,9-diazaspiro[5.5]undecane,
3-(4-chlorobenzoyl)-9-[(6-isobutoxypyridin-2-yl)methyl]-3,9-diazaspiro[5.5]undecane,
2-(4-chlorobenzoyl)-7-(3-phenoxybenzyl)-2,7-diazaspiro[3.5]nonane,
2-benzoyl-7-(3-phenoxybenzyl)-2,7-diazaspiro[3.5]nonane,
3-(2-isobutoxybenzyl)-9-(pyridazin-3-ylcarbonyl)-3,9-diazaspiro[5.5]undecane,
3-(2-isobutoxybenzyl)-9-(pyridazin-4-ylcarbonyl)-3,9-diazaspiro[5.5]undecane,
3-(2-isobutoxybenzyl)-9-(pyridin-2-ylcarbonyl)-3,9-diazaspiro[5.5]undecane,
3-(2-isobutoxybenzyl)-9-(pyridin-3-ylcarbonyl)-3,9-diazaspiro[5.5]undecane,
3-(4-chlorobenzoyl)-9-[3-(pyridin-2-ylmethoxy)benzyl]-3,9-diazaspiro[5.5]undecane,
3-(4-chlorobenzoyl)-9-[3-(pyridin-3-ylmethoxy)benzyl]-3,9-diazaspiro[5.5]undecane,
3-(3-furoyl)-9-(2-isobutoxybenzyl)-3,9-diazaspiro[5.5]undecane,
3-(2-isobutoxybenzyl)-9-(3-thienylcarbonyl)-3,9-diazaspiro[5.5]undecane,
3-(4-chlorobenzoyl)-9-(2-isobutoxybenzyl)-3,9-diazaspiro[5.5]undecane,
3-benzoyl-9-(2-isobutoxybenzyl)-3,9-diazaspiro[5.5]undecane,
2-(3-furoyl)-8-(2-isobutoxybenzyl)-2,8-diazaspiro[4.5]decane,
2-{[8-(2-isobutoxybenzyl)-2,8-diazaspiro[4.5]dec-2-yl]carbonyl}quinoline,
2-{[2-(2-isobutoxybenzyl)-2,8-diazaspiro[4.5]dec-8-yl]carbonyl}quinoline,
8-(2-isobutoxybenzyl)-2-(pyridin-2-ylacetyl)-2,8-diazaspiro[4.5]decane,
2-(4-chlorobenzoyl)-7-(2-isobutoxybenzyl)-2,7-diazaspiro[3.5]nonane,
2-(4-chlorobenzoyl)-7-(2-phenoxybenzyl)-2,7-diazaspiro[3.5]nonane,
3-[(5-chloro-2-thienyl)carbonyl]-9-(2-isobutoxybenzyl)-3,9-diazaspiro[5.5]undecane,
3-(2-isobutoxybenzyl)-9-(1H-pyrrol-2-ylcarbonyl)-3,9-diazaspiro[5.5]undecane,
3-(2-isobutoxybenzyl)-9-[4-(1,3-oxazol-5-yl)benzoyl]-3,9-diazaspiro[5.5]undecane,
3-(2-isobutoxybenzyl)-9-[4-(1H-1,2,4-triazol-1-yl)benzoyl]-3,9-diazaspiro[5.5]undecane,
3-(4-chlorobenzoyl)-9-(3-isobutoxybenzyl)-3,9-diazaspiro[5.5]undecane,
3-(2-isobutoxybenzyl)-9-[(5-methyl-2-thienyl)carbonyl]-3,9-diazaspiro[5.5]undecane,
3-(4-chlorobenzoyl)-9-[(3-phenoxy-2-thienyl)methyl]-3,9-diazaspiro[5.5]undecane,
3-(2-isobutoxybenzyl)-9-[4-(trifluoromethyl)benzoyl]-3,9-diazaspiro[5.5]undecane,
and pharmaceutically acceptable salts and solvates thereof.

20. A compound selected from the group consisting of:
3-[(6-chloropyridin-2-yl)carbonyl]-9-(2-isobutoxybenzyl)-3,9-diazaspiro[5.5]undecane,
3-(2-isobutoxybenzyl)-9-[(6-methylpyridin-3-yl)carbonyl]-3,9-diazaspiro[5.5]undecane,
3-[(6-chloropyridin-3-yl)carbonyl]-9-(2-isobutoxybenzyl)-3,9-diazaspiro[5.5]undecane,
3-(2-chloroisonicotinoyl)-9-(2-isobutoxybenzyl)-3,9-diazaspiro[5.5]undecane,
3-(2-isobutoxybenzyl)-9-(quinolin-2-ylcarbonyl)-3,9-diazaspiro[5.5]undecane,
2-[3-(4-chlorophenyl)propanoyl]-7-(2-phenoxybenzyl)-2,7-diazaspiro[3.5]nonane,
3-(2-isobutoxybenzyl)-9-[(1-oxidopyridin-3-yl)carbonyl]-3,9-diazaspiro[5.5]undecane,
3-[3-(pyridin-4-ylmethoxy)benzyl]-9-(pyrimidin-4-ylcarbonyl)-3,9-diazaspiro[5.5]undecane,
2-(4-chlorobenzoyl)-9-(2-isobutoxybenzyl)-2,9-diazaspiro[5.5]undecane,
9-(2-isobutoxybenzyl)-2-isonicotinoyl-2,9-diazaspiro[5.5]undecane,
2-(3-furoyl)-9-(2-isobutoxybenzyl)-2,9-diazaspiro[5.5]undecane,
9-(2-isobutoxybenzyl)-2-(quinolin-2-ylcarbonyl)-2,9-diazaspiro[5.5]undecane,
9-(2-isobutoxybenzyl)-2-(pyridin-4-ylacetyl)-2,9-diazaspiro[5.5]undecane,
7-(4-chlorobenzoyl)-2-(2-isobutoxybenzyl)-2,7-diazaspiro[4.5]decane,
2-(2-isobutoxybenzyl)-7-isonicotinoyl-2,7-diazaspiro[4.5]decane,
7-(3-furoyl)-2-(2-isobutoxybenzyl)-2,7-diazaspiro[4.5]decane,
2-{[2-(2-isobutoxybenzyl)-2,7-diazaspiro[4.5]dec-7-yl]carbonyl}quinoline,
2-(2-isobutoxybenzyl)-7-(pyridin-4-ylacetyl)-2,7-diazaspiro[4.5]decane,
2-(4-chlorobenzoyl)-7-(2-isobutoxybenzyl)-2,7-diazaspiro[4.4]nonane,
2-(2-isobutoxybenzyl)-7-isonicotinoyl-2,7-diazaspiro[4.4]nonane,
2-(3-furoyl)-7-(2-isobutoxybenzyl)-2,7-diazaspiro[4.4]nonane,
2-{[7-(2-isobutoxybenzyl)-2,7-diazaspiro[4.4]non-2-yl]carbonyl}quinoline,
2-(2-isobutoxybenzyl)-7-(pyridin-4-ylacetyl)-2,7-diazaspiro[4.4]nonane,
2-[(4-chlorophenyl)acetyl]-7-(2-isobutoxybenzyl)-2,7-diazaspiro[3.5]nonane,
2-[3-(4-chlorophenyl)propanoyl]-7-(3-phenoxybenzyl)-2,7-diazaspiro[3.5]nonane,
2-[3-(4-chlorophenyl)propanoyl]-7-(2-isobutoxybenzyl)-2,7-diazaspiro[3.5]nonane,
2-[(4-chlorophenyl)acetyl]-7-(2-isobutoxybenzyl)-2,7-diazaspiro[3.5]nonane,
2-(4-chlorobenzoyl)-7-(3-isobutoxybenzyl)-2,7-diazaspiro[4.4]nonane,
2-(4-chlorobenzoyl)-7-(2-phenoxybenzyl)-2,7-diazaspiro[4.4]nonane,
2-[2-(benzyloxy)benzyl]-7-(4-chlorobenzoyl)-2,7-diazaspiro[4.4]nonane,
3-(2-isobutoxybenzyl)-9-(quinolin-3-ylcarbonyl)-3,9-diazaspiro[5.5]undecane,
and pharmaceutically acceptable salts and solvates thereof.

21. A compound selected from the group consisting of:
3-(2-isobutoxybenzyl)-9-(pyridin-4-ylacetyl)-3,9-diazaspiro[5.5]undecane,
8-(2-isobutoxybenzyl)-2-(pyridin-3-ylacetyl)-2,8-diazaspiro[4.5]decane,
8-(2-isobutoxybenzyl)-2-(pyridin-4-ylacetyl)-2,8-diazaspiro[4.5]decane,
7-(2-isobutoxybenzyl)-2-(pyridin-2-ylacetyl)-2,7-diazaspiro[3.5]nonane,
7-(2-isobutoxybenzyl)-2-(pyridin-3-ylacetyl)-2,7-diazaspiro[3.5]nonane,
8-(2-isobutoxybenzyl)-2-(pyridin-3-ylcarbonyl)-2,8-diazaspiro[4.5]decane,
8-(2-isobutoxybenzyl)-2-isonicotinoyl-2,8-diazaspiro[4.5]decane,
7-(2-isobutoxybenzyl)-2-(pyridin-4-ylacetyl)-2,7-diazaspiro[3.5]nonane,
8-(2-isobutoxybenzyl)-2-(pyridin-2-ylcarbonyl)-2,8-diazaspiro[4.5]decane,
3-(2-isobutoxybenzyl)-9-(pyridin-2-ylacetyl)-3,9-diazaspiro[5.5]undecane,
3-(2-isobutoxybenzyl)-9-(pyridin-3-ylacetyl)-3,9-diazaspiro[5.5]undecane,
3-(2-isobutoxybenzyl)-9-[4-(2H-tetrazol-5-yl)benzoyl]-3,9-diazaspiro[5.5]undecane,
7-(2-isobutoxybenzyl)-2-(pyridin-2-ylcarbonyl)-2,7-diazaspiro[3.5]nonane,
7-(2-isobutoxybenzyl)-2-(pyridin-3-ylcarbonyl)-2,7-diazaspiro[3.5]nonane,
7-(2-isobutoxybenzyl)-2-isonicotinoyl-2,7-diazaspiro[3.5]nonane,
3-(2-isobutoxybenzyl)-9-(1-oxidoisonicotinoyl)-3,9-diazaspiro[5.5]undecane,
3-(2-isobutoxybenzyl)-9-(quinoxalin-2-ylcarbonyl)-3,9-diazaspiro[5.5]undecane,
3-[4-(1H-imidazol-1-yl)benzoyl]-9-(2-isobutoxybenzyl)-3,9-diazaspiro[5.5]undecane,
5-{[9-(2-isobutoxybenzyl)-3,9-diazaspiro[5.5]undec-3-yl]carbonyl}pyridin-2(1H)-one,
3-{[9-(2-isobutoxybenzyl)-3,9-diazaspiro[5.5]undec-3-yl]carbonyl}pyridin-2(1H)-one,
3-(2-isobutoxybenzyl)-9-[3-(2H-tetrazol-5-yl)benzoyl]-3,9-diazaspiro[5.5]undecane,
3-(2-isobutoxybenzyl)-9-(2-methylisonicotinoyl)-3,9-diazaspiro[5.5]undecane,
3-[2-(cyclopropylmethoxy)benzyl]-9-isonicotinoyl-3,9-diazaspiro[5.5]undecane,
3-[1-(2-isobutoxyphenyl)ethyl]-9-isonicotinoyl-3,9-diazaspiro[5.5]undecan,
3-[(6-isobutoxypyridin-2-yl)methyl]-9-isonicotinoyl-3,9-diazaspiro[5.5]undecane,
3-[(6-isobutoxypyridin-2-yl)methyl]-9-(pyrimidin-4-ylcarbonyl)-3,9-diazaspiro[5.5]undecane,
and pharmaceutically acceptable salts and solvates thereof.

22. A compound selected from the group consisting of:
3-isonicotinoyl-9-{2-[(2-methylprop-2-en-1-yl)oxy]benzyl}-3,9-diazaspiro[5.5]undecane,
3-isonicotinoyl-9-(2-phenoxybenzyl)-3,9-diazaspiro[5.5]undecane,
3-(2-isobutoxybenzyl)-9-[2-(2H-tetrazol-5-yl)benzoyl]-3,9-diazaspiro[5.5]undecane,
3-isonicotinoyl-9-[2-(1,1,2,2-tetrafluoroethoxy)benzyl]-3,9-diazaspiro[5.5]undecane,
4-{[9-(2-isobutoxybenzyl)-3,9-diazaspiro[5.5]undecane-3-yl]carbonyl}benzene sulfonamide,
8-(2-isobutoxybenzyl)-2-(pyridin-2-ylacetyl)-2,8-diazaspiro[4.5]decane,
3-(4-chlorobenzoyl)-9-[(2-isobutoxypyridin-3-yl)methyl]-3,9-diazaspiro[5.5]undecane,
3-[(2-isobutoxypyridin-3-yl)methyl]-9-isonicotinoyl-3,9-diazaspiro[5.5]undecane,
3-[(2-isobutoxypyridin-3-yl)methyl]-9-(pyrimidin-4-ylcarbonyl)-3,9-diazaspiro[5.5]undecane,
9-(2-isobutoxybenzyl)-N-3-thienyl-3,9-diazaspiro[5.5]undecane-3-carboxamide,
N-(4-chlorophenyl)-9-(2-isobutoxybenzyl)-3,9-diazaspiro[5.5]undecane-3-carboxamide,
9-(2-isobutoxybenzyl)-N-(2-phenylethyl)-3,9-diazaspiro[5.5]undecane-3-carboxamide,
9-(2-isobutoxybenzyl)-N-[2-(2-thienyl)ethyl]-3,9-diazaspiro[5.5]undecane-3-carboxamide,
9-(2-isobutoxybenzyl)-N-2-thienyl-3,9-diazaspiro[5.5]undecane-3-carboxamide,
N-(2,3-dihydro-1-benzofuran-6-yl)-9-(2-isobutoxybenzyl)-3,9-diazaspiro[5.5]undecane-3-carboxamide,
N-(2,3-dihydro-1,4-benzodioxin-6-yl)-9-(2-isobutoxybenzyl)-3,9-diazaspiro[5.5]undecane-3-carboxamide,
9-(2-isobutoxybenzyl)-N-(5-methyl-3-phenylisoxazol-4-yl)-3,9-diazaspiro[5.5]undecane-3-carboxamide,
9-(2-isobutoxybenzyl)-N-(3-methyl-5-phenylisoxazol-4-yl)-3,9-diazaspiro[5.5]undecane-3-carboxamide,
N-(2,6-dichloropyridin-4-yl)-9-(2-isobutoxybenzyl)-3,9-diazaspiro[5.5]undecane-3-carboxamide,
N-2,1,3-benzothiadiazol-4-yl-9-(2-isobutoxybenzyl)-3,9-diazaspiro[5.5]undecane-3-carboxamide,
9-(2-isobutoxybenzyl)-N-(4-phenoxyphenyl)-3,9-diazaspiro[5.5]undecane-3-carboxamide,
9-(2-isobutoxybenzyl)-N-(2-phenylcyclopropyl)-3,9-diazaspiro[5.5]undecane-3-carboxamide,
9-(2-isobutoxybenzyl)-N-(tetrahydro-2H-pyran-2-yl)-3,9-diazaspiro[5.5]undecane-3-carboxamide,
and pharmaceutically acceptable salts and solvates thereof.

23. A compound selected from the group consisting of:
9-(2-isobutoxybenzyl)-N-(phenyl)-3,9-diazaspiro[5.5]undecane-3-carboxamide,
N-benzyl-9-(2-isobutoxybenzyl)-3,9-diazaspiro[5.5]undecane-3-carboxamide,
N-cyclohexyl-9-(2-isobutoxybenzyl)-3,9-diazaspiro[5.5]undecane-3-carboxamide,
N-(tert-butyl)-9-(2-isobutoxybenzyl)-3,9-diazaspiro[5.5]undecane-3-carboxamide,
ethyl N-{[9-(2-isobutoxybenzyl)-3,9-diazaspiro[5.5]undec-3-yl]carbonyl}glycinate,
N-cyclopentyl-9-(2-isobutoxybenzyl)-3,9-diazaspiro[5.5]undecane-3-carboxamide,
N-(2,4-dichlorobenzyl)-9-(2-isobutoxybenzyl)-3,9-diazaspiro[5.5]undecane-3-carboxamide,
9-(2-isobutoxybenzyl)-N-(2-methoxyphenyl)-3,9-diazaspiro[5.5]undecane-3-carboxamide,
9-(2-isobutoxybenzyl)-N-(4-methoxyphenyl)-3,9-diazaspiro[5.5]undecane-3-carboxamide,
ethyl 4-({[9-(2-isobutoxybenzyl)-3,9-diazaspiro[5.5]undec-3-yl]carbonyl}amino)benzoate,
ethyl 3-({[9-(2-isobutoxybenzyl)-3,9-diazaspiro[5.5]undec-3-yl]carbonyl}amino)benzoate,
N-(3-chlorophenyl)-9-(2-isobutoxybenzyl)-3,9-diazaspiro[5.5]undecane-3-carboxamide,
9-(2-isobutoxybenzyl)-N-(4-methoxybenzyl)-3,9-diazaspiro[5.5]undecane-3-carboxamide,
N-[2-(4-ethylphenyl)ethyl]-9-(2-isobutoxybenzyl)-3,9-diazaspiro[5.5]undecane-3-carboxamide,
9-(2-isobutoxybenzyl)-N-(4-isopropylphenyl)-3,9-diazaspiro[5.5]undecane-3-carboxamide,
N-(3-cyanophenyl)-9-(2-isobutoxybenzyl)-3,9-diazaspiro[5.5]undecane-3-carboxamide,
9-(2-isobutoxybenzyl)-N-(2-methylphenyl)-3,9-diazaspiro[5.5]undecane-3-carboxamide,
9-(2-isobutoxybenzyl)-N-(3-methylphenyl)-3,9-diazaspiro[5.5]undecane-3-carboxamide,
9-(2-isobutoxybenzyl)-N-(4-methylphenyl)-3,9-diazaspiro[5.5]undecane-3-carboxamide,
N-(2,6-dichlorophenyl)-9-(2-isobutoxybenzyl)-3,9-diazaspiro[5.5]undecane-3-carboxamide,
N-(3,4-dichlorophenyl)-9-(2-isobutoxybenzyl)-3,9-diazaspiro[5.5]undecane-3-carboxamide,
N-(3,5-dichlorophenyl)-9-(2-isobutoxybenzyl)-3,9-diazaspiro[5.5]undecane-3-carboxamide,
N-(4-chlorophenyl)-9-(2-isobutoxybenzyl)-2,9-diazaspiro[5.5]undecane-2-carboxamide,
N-(4-chlorophenyl)-2-(2-isobutoxybenzyl)-2,7-diazaspiro[4.5]decane-7-carboxamide,
N-(4-chlorophenyl)-7-(2-isobutoxybenzyl)-2,7-diazaspiro[4.4]nonane-2-carboxamide,
N-(4-chlorophenyl)-7-(2-isobutoxybenzyl)-2,7-diazaspiro[3.5]nonane-2-carboxamide,
9-(2-isobutoxybenzyl)-N-[(4-methylphenyl)sulfonyl]-3,9-diazaspiro[5.5]undecane-3-carboxamide,
N-[(4-chlorophenyl)sulfonyl]-9-(2-isobutoxybenzyl)-3,9-diazaspiro[5.5]undecane-3-carboxamide,
9-(2-isobutoxybenzyl)-N-[(2-methylphenyl)sulfonyl]-3,9-diazaspiro[5.5]undecane-3-carboxamide,
N-[(4-chlorophenyl)sulfonyl]-9-(2-isobutoxybenzyl)-2,9-diazaspiro[5.5]undecane-2-carboxamide,
3-(2-isobutoxybenzyl)-9-(2-thienylsulfonyl)-3,9-diazaspiro[5.5]undecane,
3-(2-isobutoxybenzyl)-9-(phenylsulfonyl)-3,9-diazaspiro[5.5]undecane,
3-(2-isobutoxybenzyl)-9-(propylsulfonyl)-3,9-diazaspiro[5.5]undecane,
3-(2-isobutoxybenzyl)-9-[(3-methylphenyl)sulfonyl]-3,9-diazaspiro[5.5]undecane,
3-(benzylsulfonyl)-9-(2-isobutoxybenzyl)-3,9-diazaspiro[5.5]undecane,
3-(2-isobutoxybenzyl)-9-(isopropylsulfonyl)-3,9-diazaspiro[5.5]undecane,
3-(2-isobutoxybenzyl)-9-(3-thienylsulfonyl)-3,9-diazaspiro[5.5]undecane,
3-[(2,5-dimethyl-3-furyl)sulfonyl]-9-(2-isobutoxybenzyl)-3,9-diazaspiro[5.5]undecane,
3-[(3,5-dimethylisoxazol-4-yl)sulfonyl]-9-(2-isobutoxybenzyl)-3,9-diazaspiro[5.5]undecane,
and pharmaceutically acceptable salts and solvates thereof.

24. A compound selected from the group consisting of:
2-{[9-(2-isobutoxybenzyl)-3,9-diazaspiro[5.5]undec-3-yl]sulfonyl}benzonitrile,
4-{[9-(2-isobutoxybenzyl)-3,9-diazaspiro[5.5]undec-3-yl]sulfonyl}benzonitrile,
3-[(2,5-dimethoxyphenyl)sulfonyl]-9-(2-isobutoxybenzyl)-3,9-diazaspiro[5.5]undecane,
3-(2-isobutoxybenzyl)-9-[(4-methoxyphenyl)sulfonyl]-3,9-diazaspiro[5.5]undecane,
3-(2-isobutoxybenzyl)-9-[(3-nitrophenyl)sulfonyl]-3,9-diazaspiro[5.5]undecane,
3-[(2-chlorophenyl)sulfonyl]-9-(2-isobutoxybenzyl)-3,9-diazaspiro[5.5]undecane,
3-[(4-chlorophenyl)sulfonyl]-9-(2-isobutoxybenzyl)-3,9-diazaspiro[5.5]undecane,
3-[(2,4-dimethyl-1,3-thiazol-5-yl)sulfonyl]-9-(2-isobutoxybenzyl)-3,9-diazaspiro[5.5]undecane,
3-(2,1,3-benzoxadiazol-4-ylsulfonyl)-9-(2-isobutoxybenzyl)-3,9-diazaspiro[5.5]undecane,
2-[(4-chlorophenyl)sulfonyl]-9-(2-isobutoxybenzyl)-2,9-diazaspiro[5.5]undecane,
7-[(4-chlorophenyl)sulfonyl]-2-(2-isobutoxybenzyl)-2,7-diazaspiro[4.5]decane,
2-[(4-chlorophenyl)sulfonyl]-7-(2-isobutoxybenzyl)-2,7-diazaspiro[4.4]nonane,
2-[(4-chlorophenyl)sulfonyl]-7-(2-isobutoxybenzyl)-2,7-diazaspiro[3.5]nonane,
3-(2-isobutoxybenzyl)-9-[(4-isopropylphenyl)sulfonyl]-3,9-diazaspiro[5.5]undecane,
4-{[9-(2-isobutoxybenzyl)-3,9-diazaspiro[5.5]undec-3-yl]sulfonyl}benzoic acid,
3-(2-isobutoxybenzyl)-9-(quinolin-8-ylsulfonyl)-3,9-diazaspiro[5.5]undecane,
3-[(5-chloro-1,3-dimethyl-1H-pyrazol-4-yl)sulfonyl]-9-(2-isobutoxybenzyl)-3,9-diazaspiro[5.5]undecane,
3-[(4-tert-butylphenyl)sulfonyl]-9-(2-isobutoxybenzyl)-3,9-diazaspiro[5.5]undecane,
N-(4-{[9-(2-isobutoxybenzyl)-3,9-diazaspiro[5.5]undec-3-yl]sulfonyl}phenyl)acetamide,
3-(2,1,3-benzothiadiazol-4-ylsulfonyl)-9-(2-isobutoxybenzyl)-3,9-diazaspiro[5.5]undecane,
2-hydroxy-5-{[9-(2-isobutoxybenzyl)-3,9-diazaspiro[5.5]undec-3-yl]sulfonyl}benzoic acid,
methyl 3-{[9-(2-isobutoxybenzyl)-3,9-diazaspiro[5.5]undec-3-yl]sulfonyl}thiophene-2-carboxylate,
3-{[4-(2-furyl)phenyl]sulfonyl}-9-(2-isobutoxybenzyl)-3,9-diazaspiro[5.5]undecane,
3-(2-isobutoxybenzyl)-9-[(4-methyl-3,4-dihydro-2H-1,4-benzoxazin-7-yl)sulfonyl]-3,9-diazaspiro[5.5]undecane,
3-(2-isobutoxybenzyl)-9-[(5-methyl-1-phenyl-1H-pyrazol-4-yl)sulfonyl]-3,9-diazaspiro[5.5]undecane,
3-(2-isobutoxybenzyl)-9-[(6-morpholin-4-ylpyridin-3-yl)sulfonyl]-3,9-diazaspiro[5.5]undecane,
3-(2,3-dihydro-1-benzofuran-5-ylsulfonyl)-9-(2-isobutoxybenzyl)-3,9-diazaspiro[5.5]undecane,
3-{[9-(2-isobutoxybenzyl)-3,9-diazaspiro[5.5]undec-3-yl]carbonyl}benzoic acid,
4-{2-[9-(2-isobutoxybenzyl)-3,9-diazaspiro[5.5]undec-3-yl]-2-oxoethyl}benzoic acid,
2-{[9-(2-isobutoxybenzyl)-3,9-diazaspiro[5.5]undec-3-yl]carbonyl}benzoic acid,
(2-{[9-(2-isobutoxybenzyl)-3,9-diazaspiro[5.5]undec-3-yl]carbonyl}phenyl)acetic acid,
(3-{[9-(2-isobutoxybenzyl)-3,9-diazaspiro[5.5]undec-3-yl]carbonyl}phenyl)acetic acid,
[{2-[9-(2-isobutoxybenzyl)-3,9-diazaspiro[5.5]undec-3-yl]-2-oxoethyl}(phenyl)amino]acetic acid,
5-{[9-(2-isobutoxybenzyl)-3,9-diazaspiro[5.5]undec-3-yl]carbonyl}thiophene-2-carboxylic acid,
(2E,4E)-6-[9-(2-isobutoxybenzyl)-3,9-diazaspiro[5.5]undec-3-yl]-6-oxohexa-2,4-dienoic acid,
6-[9-(2-isobutoxybenzyl)-3,9-diazaspiro[5.5]undec-3-yl]-6-oxohexanoic acid,
4′-{[9-(2-isobutoxybenzyl)-3,9-diazaspiro[5.5]undec-3-yl]carbonyl}biphenyl-4-carboxylic acid,
(3-{2-[9-(2-isobutoxybenzyl)-3,9-diazaspiro[5.5]undec-3-yl]-2-oxoethyl}phenyl)acetic acid,
3-{[9-(2-isobutoxybenzyl)-3,9-diazaspiro[5.5]undec-3-yl]carbonyl}-1H-pyrazole-5-carboxylic acid,
{2-[9-(2-isobutoxybenzyl)-3,9-diazaspiro[5.5]undec-3-yl]-2-oxoethoxy}acetic acid,
3-(4-chlorobenzoyl)-9-{2-[(2,6-dichlorobenzyl)oxy]benzyl}-3,9-diazaspiro[5.5]undecane,
3-(4-chlorobenzoyl)-9-[2-(2-methoxyphenoxy)benzyl]-3,9-diazaspiro[5.5]undecane,
3-[2-(tert-butylthio)benzyl]-9-(4-chlorobenzoyl)-3,9-diazaspiro[5.5]undecane,
3-(4-chlorobenzoyl)-9-[3-(pyridin-2-yloxy)benzyl]-3,9-diazaspiro[5.5]undecane,
3-(4-chlorobenzoyl)-9-[(3,5-diethoxypyridin-4-yl)methyl]-3,9-diazaspiro[5.5]undecane,
2-(2-{[9-(4-chlorobenzoyl)-3,9-diazaspiro[5.5]undec-3-yl]methyl}phenoxy)benzonitrile,
3-[2-(allyloxy)benzyl]-9-(4-chlorobenzoyl)-3,9-diazaspiro[5.5]undecane,
3-[3-(benzyloxy)benzyl]-9-(4-chlorobenzoyl)-3,9-diazaspiro[5.5]undecane,
3-(4-chlorobenzoyl)-9-(4-phenoxybenzyl)-3,9-diazaspiro[5.5]undecane,
3-(4-chlorobenzoyl)-9-[2-(4-methylphenoxy)benzyl]-3,9-diazaspiro[5.5]undecane,
3-[2-(4-tert-butylphenoxy)benzyl]-9-(4-chlorobenzoyl)-3,9-diazaspiro[5.5]undecane,
3-(4-chlorobenzoyl)-9-[2-(3-chlorophenoxy)benzyl]-3,9-diazaspiro[5.5]undecane,
3-(4-chlorobenzoyl)-9-[2-(4-fluorophenoxy)benzyl]-3,9-diazaspiro[5.5]undecane,
3-(4-chlorobenzoyl)-9-{2-[3-(trifluoromethyl)phenoxy]benzyl}-3,9-diazaspiro[5.5]undecane,
3-(4-chlorobenzoyl)-9-[2-(2,4-dichlorophenoxy)benzyl]-3,9-diazaspiro[5.5]undecane,
3-(4-chlorobenzoyl)-9-{2-[(2-fluorophenyl)thio]benzyl}-3,9-diazaspiro[5.5]undecane,
3-(4-chlorobenzoyl)-9-(4-fluoro-3-phenoxybenzyl)-3,9-diazaspiro[5.5]undecane,
3-(4-chlorobenzoyl)-9-(2-isobutoxybenzyl)-3,9-diazaspiro[5.5]undecane,
2-[2-(allyloxy)benzyl]-7-(4-chlorobenzoyl)-2,7-diazaspiro[3.5]nonane,
7-(4-chlorobenzoyl)-2-[2-(3-chlorophenoxy)benzyl]-2,7-diazaspiro[3.5]nonane,
7-(4-chlorobenzoyl)-2-[2-(4-fluorophenoxy)benzyl]-2,7-diazaspiro[3.5]nonane,
7-(4-chlorobenzoyl)-2-{2-[3-(trifluoromethyl)phenoxy]benzyl}-2,7-diazaspiro[3.5]nonane,
and pharmaceutically acceptable salts and solvates thereof.

25. A compound selected from the group consisting of:
2-(2-{[7-(4-chlorobenzoyl)-2,7-diazaspiro[3.5]non-2-yl]methyl}phenoxy)benzonitrile,
7-(4-chlorobenzoyl)-2-[2-(pyridin-3-yloxy)benzyl]-2,7-diazaspiro[3.5]nonane,
7-(4-chlorobenzoyl)-2-(4-fluoro-3-phenoxybenzyl)-2,7-diazaspiro[3.5]nonane,
7-(4-chlorobenzoyl)-2-(2-isobutoxybenzyl)-2,7-diazaspiro[3.5]nonane,
7-[2-(allyloxy)benzyl]-2-(4-chlorobenzoyl)-2,7-diazaspiro[3.5]nonane,
7-[3-(benzyloxy)benzyl]-2-(4-chlorobenzoyl)-2,7-diazaspiro[3.5]nonane,
2-(4-chlorobenzoyl)-7-[2-(3-chlorophenoxy)benzyl]-2,7-diazaspiro[3.5]nonane,
2-(4-chlorobenzoyl)-7-[2-(4-fluorophenoxy)benzyl]-2,7-diazaspiro[3.5]nonane,
2-(4-chlorobenzoyl)-7-{2-[3-(trifluoromethyl)phenoxy]benzyl}-2,7-diazaspiro[3.5]nonane,
2-(2-{[2-(4-chlorobenzoyl)-2,7-diazaspiro[3.5]non-7-yl]methyl}phenoxy)benzonitrile,
2-(4-chlorobenzoyl)-7-[2-(pyridin-3-yloxy)benzyl]-2,7-diazaspiro[3.5]nonane,
2-(4-chlorobenzoyl)-7-(4-fluoro-3-phenoxybenzyl)-2,7-diazaspiro[3.5]nonane,
2-(4-chlorobenzoyl)-7-(2-isobutoxybenzyl)-2,7-diazaspiro[3.5]nonane,
8-[2-(allyloxy)benzyl]-2-(4-chlorobenzoyl)-2,8-diazaspiro[4.5]decane,
8-[3-(benzyloxy)benzyl]-2-(4-chlorobenzoyl)-2,8-diazaspiro[4.5]decane,
2-(4-chlorobenzoyl)-8-(4-phenoxybenzyl)-2,8-diazaspiro[4.5]decane,
2-(4-chlorobenzoyl)-8-[2-(3-chlorophenoxy)benzyl]-2,8-diazaspiro[4.5]decane,
2-(4-chlorobenzoyl)-8-[2-(4-fluorophenoxy)benzyl]-2,8-diazaspiro[4.5]decane,
2-(4-chlorobenzoyl)-8-{2-[3-(trifluoromethyl)phenoxy]benzyl}-2,8-diazaspiro[4.5]decane,
2-(4-chlorobenzoyl)-8-[2-(2,4-dichlorophenoxy)benzyl]-2,8-diazaspiro[4.5]decane,
2-(2-{[2-(4-chlorobenzoyl)-2,8-diazaspiro[4.5]dec-8-yl]methyl}phenoxy)benzonitrile,
2-(4-chlorobenzoyl)-8-(4-fluoro-3-phenoxybenzyl)-2,8-diazaspiro[4.5]decane,
2-(4-chlorobenzoyl)-8-(2-isobutoxybenzyl)-2,8-diazaspiro[4.5]decane,
2-[2-(allyloxy)benzyl]-8-(4-chlorobenzoyl)-2,8-diazaspiro[4.5]decane,
2-[3-(benzyloxy)benzyl]-8-(4-chlorobenzoyl)-2,8-diazaspiro[4.5]decane,
8-(4-chlorobenzoyl)-2-[2-(3-chlorophenoxy)benzyl]-2,8-diazaspiro[4.5]decane,
8-(4-chlorobenzoyl)-2-[2-(4-fluorophenoxy)benzyl]-2,8-diazaspiro[4.5]decane,
8-(4-chlorobenzoyl)-2-{2-[3-(trifluoromethyl)phenoxy]benzyl}-2,8-diazaspiro[4.5]decane,
2-(2-{[8-(4-chlorobenzoyl)-2,8-diazaspiro[4.5]dec-2-yl]methyl}phenoxy)benzonitrile,
8-(4-chlorobenzoyl)-2-{2-[(2-chloro-1,3-thiazol-5-yl)methoxy]benzyl}-2,8-diazaspiro[4.5]decane,
8-(4-chlorobenzoyl)-2-(4-fluoro-3-phenoxybenzyl)-2,8-diazaspiro[4.5]decane,
8-(4-chlorobenzoyl)-2-(2-isobutoxybenzyl)-2,8-diazaspiro[4.5]decane,
3-(4-chlorobenzoyl)-9-[2-(3-methylbutoxy)benzyl]-3,9-diazaspiro[5.5]undecane,
3-(2-ethoxybenzyl)-9-(4-fluorobenzoyl)-3,9-diazaspiro[5.5]undecane,
3-(2-ethoxybenzyl)-9-(4-nitrobenzoyl)-3,9-diazaspiro[5.5]undecane,
3-(4-chlorobenzoyl)-9-(2-isobutoxybenzyl)-3,9-diazaspiro[5.5]undecane,
3-(2-isobutoxybenzyl)-9-(4-nitrobenzoyl)-3,9-diazaspiro[5.5]undecane,
3-(4-fluorobenzoyl)-9-(2-isobutoxybenzyl)-3,9-diazaspiro[5.5]undecane,
2-chloro-5-{[9-(2-isobutoxybenzyl)-3,9-diazaspiro[5.5]undec-3-yl]carbonyl}benzenesulfonamide,
and pharmaceutically acceptable salts and solvates thereof.

26. A compound selected from the group consisting of:
3-(2-isobutoxybenzyl)-9-(1H-pyrrol-3-ylcarbonyl)-3,9-diazaspiro[5.5]undecane,
8-(2-isobutoxybenzyl)-2-[2-(methylsulfonyl)benzoyl]-2,8-diazaspiro[4.5]decane,
2-[4-chloro-2-(methylsulfonyl)benzoyl]-8-(2-isobutoxybenzyl)-2,8-diazaspiro[4.5]decane,
2-{[8-(2-isobutoxybenzyl)-2,8-diazaspiro[4.5]dec-2-yl]carbonyl}nicotinamide,
8-(2-isobutoxybenzyl)-2-[(2-morpholin-4-ylpyridin-3-yl)carbonyl]-2,8-diazaspiro[4.5]decane,
2-[(2,6-dimethoxypyridin-3-yl)carbonyl]-8-(2-isobutoxybenzyl)-2,8-diazaspiro[4.5]decane,
2-(2,4-dimethoxybenzoyl)-8-(2-isobutoxybenzyl)-2,8-diazaspiro[4.5]decane,
3-[(4-chlorobenzyl)sulfonyl]-9-(2-ethoxybenzyl)-3,9-diazaspiro[5.5]undecane,
8-(2-isobutoxybenzyl)-2-[4-(methylsulfonyl)benzoyl]-2,8-diazaspiro[4.5]decane,
8-(2-isobutoxybenzyl)-2-[2-methoxy-4-(methylthio)benzoyl]-2,8-diazaspiro[4.5]decane,
2-(4-butoxybenzoyl)-8-(2-isobutoxybenzyl)-2,8-diazaspiro[4.5]decane,
1-(4-{[8-(2-isobutoxybenzyl)-2,8-diazaspiro[4.5]dec-2-yl]carbonyl}phenyl)ethanone,
2-(4-ethylbenzoyl)-8-(2-isobutoxybenzyl)-2,8-diazaspiro[4.5]decane,
2-{[8-(2-isobutoxybenzyl)-2,8-diazaspiro[4.5]dec-2-yl]carbonyl}quinoline,
2-(4-chloro-2-methoxybenzoyl)-8-(2-isobutoxybenzyl)-2,8-diazaspiro[4.5]decane,
8-(2-isobutoxybenzyl)-2-(4-morpholin-4-ylbenzoyl)-2,8-diazaspiro[4.5]decane,
8-(2-isobutoxybenzyl)-2-(6-methoxy-2-naphthoyl)-2,8-diazaspiro[4.5]decane,
2-(2,3-dichlorobenzoyl)-8-(2-isobutoxybenzyl)-2,8-diazaspiro[4.5]decane,
8-(2-isobutoxybenzyl)-2-(3-methoxybenzoyl)-2,8-diazaspiro[4.5]decane,
2-(2,3-dimethylbenzoyl)-8-(2-isobutoxybenzyl)-2,8-diazaspiro[4.5]decane,
8-(2-isobutoxybenzyl)-2-(4-methylbenzoyl)-2,8-diazaspiro[4.5]decane,
2-(3,4-dichlorobenzoyl)-8-(2-isobutoxybenzyl)-2,8-diazaspiro[4.5]decane,
2-(2,4-dichlorobenzoyl)-8-(2-isobutoxybenzyl)-2,8-diazaspiro[4.5]decane,
8-(2-isobutoxybenzyl)-2-(4-isopropoxybenzoyl)-2,8-diazaspiro[4.5]decane,
8-(2-isobutoxybenzyl)-2-(4-phenoxybenzoyl)-2,8-diazaspiro[4.5]decane,
8-(2-isobutoxybenzyl)-2-(2-naphthoyl)-2,8-diazaspiro[4.5]decane,
2-(2-chlorobenzoyl)-8-(2-isobutoxybenzyl)-2,8-diazaspiro[4.5]decane,
2-(2,3-dimethoxybenzoyl)-8-(2-isobutoxybenzyl)-2,8-diazaspiro[4.5]decane,
8-(2-isobutoxybenzyl)-2-(1-naphthoyl)-2,8-diazaspiro[4.5]decane,
8-(2-isobutoxybenzyl)-2-(4-methoxybenzoyl)-2,8-diazaspiro[4.5]decane,
N,N-diethyl-4-{[8-(2-isobutoxybenzyl)-2,8-diazaspiro[4.5]dec-2-yl]carbonyl}aniline,
8-(2-isobutoxybenzyl)-2-(4-propylbenzoyl)-2,8-diazaspiro[4.5]decane,
and pharmaceutically acceptable salts and solvates thereof.

27. A compound selected from the group consisting of:
8-(2-isobutoxybenzyl)-2-[3-(trifluoromethyl)benzoyl]-2,8-diazaspiro[4.5]decane,
8-(2-isobutoxybenzyl)-2-[4-(trifluoromethyl)benzoyl]-2,8-diazaspiro[4.5]decane,
4-{[8-(2-isobutoxybenzyl)-2,8-diazaspiro[4.5]dec-2-yl]carbonyl}quinoline,
2-(3-chloro-2-methylbenzoyl)-8-(2-isobutoxybenzyl)-2,8-diazaspiro[4.5]decane,
(4-{2-[8-(2-isobutoxybenzyl)-2,8-diazaspiro[4.5]dec-2-yl]-2-oxoethyl}phenyl)dimethylamine,
2-[(2-fluorophenyl)acetyl]-8-(2-isobutoxybenzyl)-2,8-diazaspiro[4.5]decane,
8-(2-isobutoxybenzyl)-2-[(3-nitrophenyl)acetyl]-2,8-diazaspiro[4.5]decane,
8-(2-isobutoxybenzyl)-2-[(4-nitrophenyl)acetyl]-2,8-diazaspiro[4.5]decane,
8-(2-isobutoxybenzyl)-2-[(2-nitrophenyl)acetyl]-2,8-diazaspiro[4.5]decane,
2-[(3,4-dimethoxyphenyl)acetyl]-8-(2-isobutoxybenzyl)-2,8-diazaspiro[4.5]decane,
2-(3-furoyl)-8-(2-isobutoxybenzyl)-2,8-diazaspiro[4.5]decane,
2-[(4-chlorophenyl)acetyl]-8-(2-isobutoxybenzyl)-2,8-diazaspiro[4.5]decane,
8-(2-isobutoxybenzyl)-2-(1,2,3-thiadiazol-4-ylcarbonyl)-2,8-diazaspiro[4.5]decane,
8-(2-isobutoxybenzyl)-2-[(5-methyl-1H-pyrazol-3-yl)carbonyl]-2,8-diazaspiro[4.5]decane,
2-[(1,5-dimethyl-1H-pyrazol-3-yl)carbonyl]-8-(2-isobutoxybenzyl)-2,8-diazaspiro[4.5]decane,
2-[(4-butoxyphenyl)acetyl]-8-(2-isobutoxybenzyl)-2,8-diazaspiro[4.5]decane,
2-[(3,5-difluorophenyl)acetyl]-8-(2-isobutoxybenzyl)-2,8-diazaspiro[4.5]decane,
2-[(2,4-dichlorophenyl)acetyl]-8-(2-isobutoxybenzyl)-2,8-diazaspiro[4.5]decane,
2-[(2,4-difluorophenyl)acetyl]-8-(2-isobutoxybenzyl)-2,8-diazaspiro[4.5]decane,
8-(2-isobutoxybenzyl)-2-[(3-methylisoxazol-4-yl)carbonyl]-2,8-diazaspiro[4.5]decane,
8-(2-isobutoxybenzyl)-2-(2-methyl-3-furoyl)-2,8-diazaspiro[4.5]decane,
8-(2-isobutoxybenzyl)-2-[(5-methylisoxazol-4-yl)carbonyl]-2,8-diazaspiro[4.5]decane,
2-(1,3-benzodioxol-5-ylacetyl)-8-(2-isobutoxybenzyl)-2,8-diazaspiro[4.5]decane,
2-[(3,5-dimethylisoxazol-4-yl)carbonyl]-8-(2-isobutoxybenzyl)-2,8-diazaspiro[4.5]decane,
8-(2-isobutoxybenzyl)-2-[(1,2,5-trimethyl-1H-pyrrol-3-yl)carbonyl]-2,8-diazaspiro[4.5]decane,
8-(2-isobutoxybenzyl)-2-[(5-nitro-1H-pyrazol-3-yl)carbonyl]-2,8-diazaspiro[4.5]decane,
2-[(2,5-difluorophenyl)acetyl]-8-(2-isobutoxybenzyl)-2,8-diazaspiro[4.5]decane,
2-{[4-(benzyloxy)-3-methoxyphenyl]acetyl}-8-(2-isobutoxybenzyl)-2,8-diazaspiro[4.5]decane,
8-(2-isobutoxybenzyl)-2-{[4-(trifluoromethoxy)phenyl]acetyl}-2,8-diazaspiro[4.5]decane,
2-(2,5-dimethyl-3-furoyl)-8-(2-isobutoxybenzyl)-2,8-diazaspiro[4.5]decane,
8-(2-isobutoxybenzyl)-2-[(4-methylphenyl)acetyl]-2,8-diazaspiro[4.5]decane,
8-(2-isobutoxybenzyl)-2-[(4-isopropylphenyl)acetyl]-2,8-diazaspiro[4.5]decane,
8-(2-isobutoxybenzyl)-2-{[4-(methylsulfonyl)phenyl]acetyl}-2,8-diazaspiro[4.5]decane,
8-(2-isobutoxybenzyl)-2-(1H-pyrazol-4-ylcarbonyl)-2,8-diazaspiro[4.5]decane,
8-(2-isobutoxybenzyl)-2-[(4-nitro-1H-pyrazol-3-yl)carbonyl]-2,8-diazaspiro[4.5]decane,
(2-{[8-(2-isobutoxybenzyl)-2,8-diazaspiro[4.5]dec-2-yl]carbonyl}phenyl)dimethylamine,
2-[(3,5-dimethylphenyl)acetyl]-8-(2-isobutoxybenzyl)-2,8-diazaspiro[4.5]decane,
2-(3-chloro-4-methylbenzoyl)-8-(2-isobutoxybenzyl)-2,8-diazaspiro[4.5]decane,
8-(2-isobutoxybenzyl)-2-[(4-methoxy-3-thienyl)carbonyl]-2,8-diazaspiro[4.5]decane,
8-(2-isobutoxybenzyl)-2-{[3-(trifluoromethyl)phenyl]acetyl}-2,8-diazaspiro[4.5]decane,
8-[(6-chloropyridin-3-yl)carbonyl]-2-(2-isobutoxybenzyl)-2,8-diazaspiro[4.5]decane,
and pharmaceutically acceptable salts and solvates thereof.

28. A compound selected from the group consisting of:
(4-{[2-(2-isobutoxybenzyl)-2,8-diazaspiro[4.5]dec-8-yl]carbonyl}phenyl)dimethylamine,
2-(2-isobutoxybenzyl)-8-[4-(methylsulfonyl)benzoyl]-2,8-diazaspiro[4.5]decane,
8-(4-butoxybenzoyl)-2-(2-isobutoxybenzyl)-2,8-diazaspiro[4.5]decane,
1-(4-{[2-(2-isobutoxybenzyl)-2,8-diazaspiro[4.5]dec-8-yl]carbonyl}phenyl)ethanone,
8-(4-ethylbenzoyl)-2-(2-isobutoxybenzyl)-2,8-diazaspiro[4.5]decane,
2-{[2-(2-isobutoxybenzyl)-2,8-diazaspiro[4.5]dec-8-yl]carbonyl}quinoline,
8-(4-chloro-2-methoxybenzoyl)-2-(2-isobutoxybenzyl)-2,8-diazaspiro[4.5]decane,
3-{[2-(2-isobutoxybenzyl)-2,8-diazaspiro[4.5]dec-8-yl]carbonyl}benzonitrile,
2-(2-isobutoxybenzyl)-8-(3-phenoxybenzoyl)-2,8-diazaspiro[4.5]decane,
8-(4-tert-butylbenzoyl)-2-(2-isobutoxybenzyl)-2,8-diazaspiro[4.5]decane,
4-{[2-(2-isobutoxybenzyl)-2,8-diazaspiro[4.5]dec-8-yl]carbonyl}benzonitrile,
2-(2-isobutoxybenzyl)-8-(4-morpholin-4-ylbenzoyl)-2,8-diazaspiro[4.5]decane,
2-(2-isobutoxybenzyl)-8-(6-methoxy-2-naphthoyl)-2,8-diazaspiro[4.5]decane,
8-(2,3-dichlorobenzoyl)-2-(2-isobutoxybenzyl)-2,8-diazaspiro[4.5]decane,
2-(2-isobutoxybenzyl)-8-(3-methoxybenzoyl)-2,8-diazaspiro[4.5]decane,
8-(2,3-dimethylbenzoyl)-2-(2-isobutoxybenzyl)-2,8-diazaspiro[4.5]decane,
2-(2-isobutoxybenzyl)-8-(4-methylbenzoyl)-2,8-diazaspiro[4.5]decane,
8-(3,4-dichlorobenzoyl)-2-(2-isobutoxybenzyl)-2,8-diazaspiro[4.5]decane,
8-(3,4-dimethoxybenzoyl)-2-(2-isobutoxybenzyl)-2,8-diazaspiro[4.5]decane,
8-(2,4-dichlorobenzoyl)-2-(2-isobutoxybenzyl)-2,8-diazaspiro[4.5]decane,
2-(2-isobutoxybenzyl)-8-(4-isopropoxybenzoyl)-2,8-diazaspiro[4.5]decane,
2-(2-isobutoxybenzyl)-8-(2-naphthoyl)-2,8-diazaspiro[4.5]decane,
8-(2-chlorobenzoyl)-2-(2-isobutoxybenzyl)-2,8-diazaspiro[4.5]decane,
8-(2,3-dimethoxybenzoyl)-2-(2-isobutoxybenzyl)-2,8-diazaspiro[4.5]decane,
2-(2-isobutoxybenzyl)-8-(1-naphthoyl)-2,8-diazaspiro[4.5]decane,
(3-{[2-(2-isobutoxybenzyl)-2,8-diazaspiro[4.5]dec-8-yl]carbonyl}phenyl)dimethylamine,
2-(2-isobutoxybenzyl)-8-(4-methoxybenzoyl)-2,8-diazaspiro[4.5]decane,
N,N-diethyl-4-{[2-(2-isobutoxybenzyl)-2,8-diazaspiro[4.5]dec-8-yl]carbonyl}aniline,
2-(2-isobutoxybenzyl)-8-(4-propylbenzoyl)-2,8-diazaspiro[4.5]decane,
8-(2-chloroisonicotinoyl)-2-(2-isobutoxybenzyl)-2,8-diazaspiro[4.5]decane,
2-(2-isobutoxybenzyl)-8-[3-(trifluoromethyl)benzoyl]-2,8-diazaspiro[4.5]decane,
2-(2-isobutoxybenzyl)-8-[4-(trifluoromethyl)benzoyl]-2,8-diazaspiro[4.5]decane,
4-{[2-(2-isobutoxybenzyl)-2,8-diazaspiro[4.5]dec-8-yl]carbonyl}quinoline,
8-(3-chloro-2-methylbenzoyl)-2-(2-isobutoxybenzyl)-2,8-diazaspiro[4.5]decane,
(4-{2-[2-(2-isobutoxybenzyl)-2,8-diazaspiro[4.5]dec-8-yl]-2-oxoethyl}phenyl)dimethylamine,
8-[(2-fluorophenyl)acetyl]-2-(2-isobutoxybenzyl)-2,8-diazaspiro[4.5]decane,
2-(2-isobutoxybenzyl)-8-[(3-nitrophenyl)acetyl]-2,8-diazaspiro[4.5]decane,
2-(2-isobutoxybenzyl)-8-[(4-nitrophenyl)acetyl]-2,8-diazaspiro[4.5]decane,
2-(2-isobutoxybenzyl)-8-[(2-nitrophenyl)acetyl]-2,8-diazaspiro[4.5]decane,
8-[(3,4-dimethoxyphenyl)acetyl]-2-(2-isobutoxybenzyl)-2,8-diazaspiro[4.5]decane,
8-(3-furoyl)-2-(2-isobutoxybenzyl)-2,8-diazaspiro[4.5]decane,
8-[(4-chlorophenyl)acetyl]-2-(2-isobutoxybenzyl)-2,8-diazaspiro[4.5]decane,
2-(2-isobutoxybenzyl)-8-(1,2,3-thiadiazol-4-ylcarbonyl)-2,8-diazaspiro[4.5]decane,
2-(2-isobutoxybenzyl)-8-[(5-methyl-1H-pyrazol-3-yl)carbonyl]-2,8-diazaspiro[4.5]decane,
8-[(1,5-dimethyl-1H-pyrazol-3-yl)carbonyl]-2-(2-isobutoxybenzyl)-2,8-diazaspiro[4.5]decane,
and pharmaceutically acceptable salts and solvates thereof.

29. A compound selected from the group consisting of:
8-[(4-butoxyphenyl)acetyl]-2-(2-isobutoxybenzyl)-2,8-diazaspiro[4.5]decane,
8-[(3,5-difluorophenyl)acetyl]-2-(2-isobutoxybenzyl)-2,8-diazaspiro[4.5]decane,
8-[(2,4-dichlorophenyl)acetyl]-2-(2-isobutoxybenzyl)-2,8-diazaspiro[4.5]decane,
8-[(2,4-difluorophenyl)acetyl]-2-(2-isobutoxybenzyl)-2,8-diazaspiro[4.5]decane,
2-(2-isobutoxybenzyl)-8-[(3-methylisoxazol-4-yl)carbonyl]-2,8-diazaspiro[4.5]decane,
2-(2-isobutoxybenzyl)-8-(2-methyl-3-furoyl)-2,8-diazaspiro[4.5]decane,
2-(2-isobutoxybenzyl)-8-[(5-methylisoxazol-4-yl)carbonyl]-2,8-diazaspiro[4.5]decane,
8-(1,3-benzodioxol-5-ylacetyl)-2-(2-isobutoxybenzyl)-2,8-diazaspiro[4.5]decane,
2-(2-isobutoxybenzyl)-8-[(1,2,5-trimethyl-1H-pyrrol-3-yl)carbonyl]-2,8-diazaspiro[4.5]decane,
2-(2-isobutoxybenzyl)-8-[(5-nitro-1H-pyrazol-3-yl)carbonyl]-2,8-diazaspiro[4.5]decane,
8-[(2,5-difluorophenyl)acetyl]-2-(2-isobutoxybenzyl)-2,8-diazaspiro[4.5]decane,
8-{[4-(benzyloxy)-3-methoxyphenyl]acetyl}-2-(2-isobutoxybenzyl)-2,8-diazaspiro[4.5]decane,
2-(2-isobutoxybenzyl)-8-{[4-(trifluoromethoxy)phenyl]acetyl}-2,8-diazaspiro[4.5]decane,
8-(2,5-dimethyl-3-furoyl)-2-(2-isobutoxybenzyl)-2,8-diazaspiro[4.5]decane,
2-(2-isobutoxybenzyl)-8-[(4-methylphenyl)acetyl]-2,8-diazaspiro[4.5]decane,
2-(2-isobutoxybenzyl)-8-(3-thienylcarbonyl)-2,8-diazaspiro[4.5]decane,
2-(2-isobutoxybenzyl)-8-(pyridin-4-ylacetyl)-2,8-diazaspiro[4.5]decane,
2-(2-isobutoxybenzyl)-8-(pyridin-2-ylacetyl)-2,8-diazaspiro[4.5]decane,
2-(2-isobutoxybenzyl)-8-[(4-isopropylphenyl)acetyl]-2,8-diazaspiro[4.5]decane,
2-(2-isobutoxybenzyl)-8-{[4-(methylsulfonyl)phenyl]acetyl}-2,8-diazaspiro[4.5]decane,
2-(2-isobutoxybenzyl)-8-(1H-pyrazol-4-ylcarbonyl)-2,8-diazaspiro[4.5]decane,
2-(2-isobutoxybenzyl)-8-[(4-nitro-1H-pyrazol-3-yl)carbonyl]-2,8-diazaspiro[4.5]decane,
(2-{[2-(2-isobutoxybenzyl)-2,8-diazaspiro[4.5]dec-8-yl]carbonyl}phenyl)dimethylamine,
and pharmaceutically acceptable salts and solvates thereof.
